(19) United States
(12) Patent Application Publication
(10) Pub. No.: US 2013/0281463 A1
(43) Pub. Date: Oct. 24, 2013

(54) DIAZA-SPIRO[5.5]UNDECANES USEFUL AS OREXIN RECEPTOR ANTAGONISTS

(71) Applicants: Sangamesh Badiger, Bangalore (IN); Dirk Behnke, Grenzach-Wyhlen (DE); Claudia Betschart, Basel (CH); Simona Cotesta, Basel (CH); Samuel Hintermann, Basel (CH); Silvio Ofner, Muenchenstein (CH); Chetan Pandit, Bangalore (IN); Bernard Lucien Roy, Fribourg (CH)

(72) Inventors: Sangamesh Badiger, Bangalore (IN); Dirk Behnke, Grenzach-Wyhlen (DE); Claudia Betschart, Basel (CH); Simona Cotesta, Basel (CH); Samuel Hintermann, Basel (CH); Silvio Ofner, Muenchenstein (CH); Chetan Pandit, Bangalore (IN); Bernard Lucien Roy, Fribourg (CH)

(73) Assignee: NOVARTIS AG, Basel (CH)

(21) Appl. No.: 13/880,994

(22) PCT Filed: Oct. 25, 2011
(86) PCT No.: PCT/EP11/68689

§ 371 Date: Jul. 8, 2013

(30) Foreign Application Priority Data
Oct. 26, 2010 (IN) 2562/DEL/2010
Jun. 8, 2011 (IN) 1620/DEL/2011

(51) Int. Cl. C07D 471/10; A61K 45/06; A61K 31/444; A61K 31/506

(52) U.S. Cl. CPC C07D 471/10; A61K 31/506; A61K 45/06; A61K 31/444
USPC 514/255.05; 544/230; 514/275; 546/16; 514/278

(57) ABSTRACT

The invention relates to compound of the formula (I), in which the substituents are as defined in the specification; in free form or in salt form; to its preparation, to its use as medicament and to medicaments comprising it.

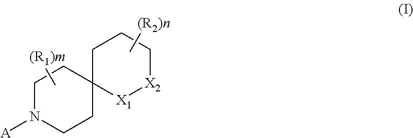

The invention relates to diaza-spiro[5.5]undecanes, to their preparation, to their use as medicaments and to medicaments comprising them.

Orexins (orexin A/OX-A and orexin B/OX-B), which are also known as hypocretins, are neuropeptides. Orexin A is a 33 amino acid peptide and orexin B is a 28 amino acid peptide (Sakurai T. et al., Cell, 1998, 92, 573-585). Orexins are produced in discrete neurons of the lateral hypothalamus and bind to G-protein-coupled receptors, the orexin receptors (also known as hypocretin receptors): known are the orexin-1 receptor (OXR1) and the orexin-2 receptor (OXR2). The orexin-1 receptor has some selectivity for OX-A, whereas the orexin-2 receptor binds OX-A and OX-B with similar affinity. Orexins regulate states of sleep and wakefulness, opening potentially novel therapeutic approaches for narcolepsy as well as insomnia and other sleep disorders (Chemelli R. M. et al., Cell, 1999, 98, 437-451). Furthermore, orexins were found to stimulate food consumption in rats suggesting a physiological role for these peptides as mediators in the central feedback mechanism that regulates feeding behavior (Sakurai T. et al., Cell, 1998, 92, 573-585). Still furthermore, orexins were shown to play a role in brain reward function/motivation suggesting usefulness to treat substance-related disorders (Harris A. C. et al, Nature, 2005, 437, 556-559). Still furthermore, it has been shown that amyloid beta levels inversely correlate with orexin levels in rodents and humans (brain and/or CSF), and that an orexin receptor antagonist reduces both amyloid beta levels and amyloid plaque load in Alzheimer's transgenic mice, thus suggesting usefulness in the treatment of Alzheimers disease (Kang J. E. et al, Science 2009, 326, 1005-1007).

Orexin receptors may have numerous implications in disorders such as

i) sleep disorders, e.g. sleep apnea, narcolepsy, insomnia, parasomnia, jet lag syndrome, disturbed biological and circadian rhythms; sleep disturbances associated with diseases such as neurological disorders, neuropathic pain and restless leg syndrome;
ii) eating disorders, e.g. appetite and taste disorders;
iii) substance-related disorders, e.g. substance abuse, substance dependence and substance withdrawal disorders, such as nicotine withdrawal or narcotics withdrawal;
iv) Alzheimers disease;
v) psychiatric, neurological and neurodegenerative disorders, e.g. depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or hemorrhagic stroke; migraine; and neurodegenerative disorders including nosological entities such as disinhibition-dementia-parkinsonism-amyotrophy complex; pallido-ponto-nigral degeneration epilepsy; seizure disorders;
vi) cardiovascular diseases, diabetes; asthma; Cushing's syndrome/disease; basophile adenoma; prolactinoma; hyperprolactinemia; hypopituitarism; hypophysis tumor/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid hemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and
vii) other diseases related to general orexin system dysfunction.

Orexin receptor antagonists are considered to be useful in the treatment of a wide range of disorders, in particular sleep disorders, eating disorders and substance-related disorders.

Therefore, there is a need to provide new orexin receptor antagonists that are good drug candidates. In particular, preferred compounds should bind potently to the orexin receptors (either as OXR1 or OXR2 subtype selective antagonists or as dual OXR1/OXR2 antagonists) whilst showing little affinity for other receptors. They should be well absorbed from the gastrointestinal tract, be sufficiently metabolically stable and possess favorable pharmacokinetic properties. When targeted against receptors in the central nervous system they should cross the blood brain barrier freely and when targeted selectively against receptors in the peripheral nervous system they should not cross the blood brain barrier. They should be non-toxic and demonstrate few side-effects. Furthermore, the ideal drug candidate will be able to exist in a physical form that is stable, non-hygroscopic and easily formulated.

The compounds of the invention are orexin receptor antagonists and are therefore potentially useful in the treatment of a wide range of disorders, particularly sleep disorders, eating disorders, substance-related disorders and Alzheimers disease.

In a first aspect, the invention relates to a compound of the formula I

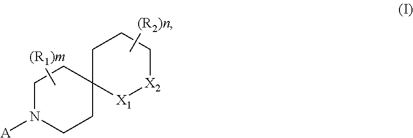

wherein
A is a five- to six-membered aromatic ring system which may contain from 1 to 4 hetero atoms selected from nitrogen, oxygen and sulfur, wherein the ring system is substituted once or more than once by R₃, and wherein a substituent on a nitrogen in a heterocyclic ring system may not be halogen;
each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—;
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the Cₘcycloalkyl may be attached directly to X₃ or via a C₁₋₂alkylene, and wherein the C₃₋₆cycloalkyl in turn may be substituted by halogen or C₁₋₄alkyl;
m is 0, 1, 2, 3, 4, 5 or 6;
n is 0, 1, 2, 3, 4, 5 or 6;
each R₁ or R₂ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

### —X₁— is —C(O)— and —X₂— is —N(L-B)—;

### or —X₁— is —N(L-B)— and —X₂— is —C(O)—;

### L is —C(R₆)₂—;

each R₆ independently is hydrogen, C₁₋₆alkyl, C₁₋₆halogenalkyl, C₃₋₇cycloalkyl or C₃₋₇cycloalkyl(C₁₋₄alkyl);
or two R₆ together with the carbon atom to which they are bound form a C₃₋₄cycloalkyl;
B is a five- to six-membered monocyclic or eight- to ten-membered fused bicyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;
each R₇ independently is C₁₋₆alkyl, C₁₋₆halogenalkyl, C₁₋₈alkoxy, C₁₋₄alkoxyC₁₋₆alkyl, C₁₋₄alkoxycarbonyl, C₁₋₆halogenalkoxy, halogen, cyano or a three- to seven-membered monocyclic ring system which may be aromatic, saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₄alkoxyC₁₋₆alkyl, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo;
in free form or in salt form or in pharmaceutically acceptable salt form.

In a second aspect, the invention relates to a compound of the formula I

wherein

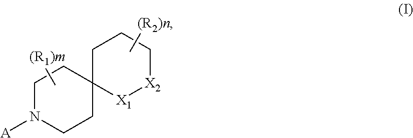

A is a five- to six-membered aromatic ring system which may contain from 1 to 4 hetero atoms selected from nitrogen, oxygen and sulfur, wherein the ring system is substituted once or more than once by R₃, and wherein a substituent on a nitrogen in a heterocyclic ring system may not be halogen;
each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—;
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m is 0, 1, 2, 3, 4, 5 or 6;
n is 0, 1, 2, 3, 4, 5 or 6;
each R₁ or R₂ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

### —X₁— is —C(O)— and —X₂— is —N(L-B)—;

### or —X₁— is —N(L-B)— and —X₂— is —C(O)—;

### L is —C(R₆)₂—;

each R₆ independently is hydrogen, C₁₋₆alkyl, C₁₋₆halogenalkyl, C₃₋₇cycloalkyl or C₃₋₇cycloalkyl(C₁₋₄alkyl);
or two R₆ together with the carbon atom to which they are bound form a C₃₋₄cycloalkyl;
B is a five- to six-membered monocyclic or eight- to ten-membered fused bicyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;
each R₇ independently is C₁₋₆alkyl, C₁₋₆halogenalkyl, C₁₋₆alkoxy, C₁₋₆halogenalkoxy, halogen, cyano or a three- to seven-membered monocyclic ring system which may be aromatic, saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo;
in free form or in salt form.

Unless specified otherwise, the term “compounds of the present invention” refers to compounds of Formula (I), prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

Unless indicated otherwise, the expressions used in this invention have the following meaning:

“Alkyl” represents a straight-chain or branched-chain alkyl group, for example, methyl, ethyl, n- or iso-propyl, n-, iso-, sec- or tert-butyl, n-pentyl, n-hexyl; C₁₋₆alkyl preferably represents a straight-chain or branched-chain C₁₋₄alkyl with particular preference given to methyl, ethyl, n-propyl, iso-propyl and tert-butyl.

Each alkyl part of “alkoxy”, “halogenalkyl” and so on shall have the same meaning as described in the above-mentioned definition of “alkyl”, especially regarding linearity and preferential size.

“C₃₋₇cycloalkyl” represents a saturated alicyclic moiety having from three to seven carbon atoms. This term refers to groups such as cyclopropyl, cyclobutyl, cyclopentyl and cyclohexyl.

A substituent being substituted “once or more than once”, for example as defined for A, is preferably substituted by one to three substituents.

Halogen is generally fluorine, chlorine, bromine or iodine; preferably fluorine, chlorine or bromine. Halogenalkyl groups preferably have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably —CF₃, —CHF₂, —CH₂F, —CHF—CH₃, —CF₂CH₃, or —CH₂CF₃.

In the context of the invention, the definition of A as “five- to six-membered monocyclic aromatic ring system which may contain from 1 to 4 heteroatoms” encompasses a C₆-aromatic hydrocarbon group or a five- to six-membered heterocyclic aromatic ring system.

In the context of the invention, the definition of B as a “five- to six-membered monocyclic or eight- to ten-membered fused biycyclic aromatic ring system” encompasses a C₆— or C₁₀-aromatic hydrocarbon group or a five-, six-, eight-, nine- or ten-membered heterocyclic aromatic ring system.

The term “fused bicyclic aromatic ring system” refers to an aromatic substituent which consists of two aromatic rings that are fused together.

In the context of the invention, the definition of R₇ as a “three- to seven-membered monocyclic ring system” encompasses a C₆-aromatic hydrocarbon group, a five- to six-membered heterocyclic aromatic ring system and a three- to seven-membered monocyclic aliphatic or heterocyclic ring system.

In the context of the invention, the definition of two R₇ as a “fused five- to seven-membered unsaturated non-aromatic ring system” encompasses five- to seven-membered hydrocarbon and heterocyclic groups which comprise at least one double-bond, which is shared with the aromatic ring system they are fused to.

A C₆- or C₁₀-aromatic hydrocarbon group is typically phenyl or naphthyl respectively. A C₆-aromatic hydrocarbon group is especially phenyl.

Preferably, but also depending on substituent definition, “five- to ten-membered heterocyclic aromatic ring systems” consist of 5 to 10 ring atoms of which 1-3 ring atoms are hetero atoms. Such heterocyclic aromatic ring systems may be present as a single aromatic ring system or as multiple, e.g. two, fused aromatic ring systems; preferably as single ring systems or as benz-annelated ring systems.

Examples of heterocyclic ring systems are: imidazo[2,1-b]thiazole, pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pteridine, and the corresponding benz-annelated heterocycles, e.g. indole, isoindole, coumarin, isoquinoline, quinoline, quinoxaline and the like. Further examples of heterocycles are: quinoxaline, indole, pyridine, 1H-benzo[d]imidazole, quinoline, pyrimidine, 1,3,4-oxadiazole, isoxazole, pyrrole or benzo[d]isoxazole.

The compounds of formula I may exist in optically active form or in form of mixtures of optical isomers, e.g. in form of racemic mixtures or diastereomeric mixtures. In particular, further asymmetrical carbon atom(s) may be present in the compounds of formula I and their salts. All optical isomers and their mixtures, including the racemic mixtures, are embraced by the invention.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the invention and includes geometric isomers. It is understood that a substituent may be attached at a chiral center of a carbon atom. Therefore, unless otherwise indicated, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. The compounds described herein may contain one or more asymmetric centers and may thus give rise to enantiomers, diastereomers, and other stereoisomeric forms that may be defined, in terms of absolute stereochemistry, as (R)— or (S)—. Unless otherwise indicated, the invention is meant to include all such possible isomers, including racemic mixtures, optically pure forms or intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

Any asymmetric atom (e.g. carbon or the like) of the compound(s) of the invention can be present in racemic or enantiomerically enriched, for example the (R)—, (S)— or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Depending on substituent definition, compounds of formula I may occur in various tautomeric forms. All tautomeric forms of the compounds of formula I are embraced by the invention.

Compounds of formula I may exist in free form or as a salt. In this specification, unless otherwise indicated, language such as “compound of formula I” is to be understood as embracing the compounds in any form, for example free or acid addition salt form. Salts which are unsuitable for pharmaceutical uses but which can be employed, for example, for the isolation or purification of free compounds of formula I, such as picrates or perchlorates, are also included. For therapeutic use, only pharmaceutically acceptable salts or free compounds are employed (where applicable in the form of pharmaceutical preparations), and are therefore preferred. Salts are preferably physiologically acceptable salts, formed by the addition of an acid.

As used herein, the term “pharmaceutically acceptable salts” refers to salts that retain the biological effectiveness and properties of the compounds of this invention and, which typically are not biologically or otherwise undesirable. The compounds of the invention may be capable of forming acid salts by virtue of the presence of suitable groups, such as amino groups.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, cam phorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

The pharmaceutically acceptable salts of the invention can be synthesized from a parent compound by conventional chemical methods. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The invention includes all pharmaceutically acceptable isotopically-labeled compounds of the invention, i.e. compounds of formula (I) wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements or an improvement in therapeutic index, and hence may be preferred in some circumstances. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula (I).

Compounds of the invention are either obtained in the free form, as a salt thereof, or as prodrug derivatives thereof.

The invention also provides pro-drugs of the compounds of the invention that converts in vivo to the compounds of the invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids (e.g., a carboxylic acid having at least one lipophilic moiety).

Exemplary prodrugs are, e.g., O-acyl derivatives of alcohols. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the invention, including their salts, can also be obtained in the form of their hydrates, or include other solvents used for their crystallization.

Preferred substituents, preferred ranges of numerical values or preferred ranges of the radicals present in compounds of the formula I, Ia, Ib and the corresponding intermediate compounds are defined below. The definition of the substituents applies to the end-products as well as to the corresponding intermediates. The definitions of the substituents may be combined at will, e.g. preferred substituents R¹ and particularly preferred substituents R².

In especially preferred embodiments, the invention relates to one or more than one of the compounds of the formula I mentioned in the Examples hereinafter, in free form or in salt form or in pharmaceutically acceptable salt form.

In one class of compounds of the invention, A is a five- to six-membered aromatic ring system which contains from 1 to 4 hetero atoms selected from nitrogen, oxygen and sulfur, wherein the ring system is substituted once or more than once by R₃, and wherein a substituent on a nitrogen in a heterocyclic ring system may not be halogen;

each R₃ independently is halogen; cyano; hydroxy; amino; or —X₃-R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is a six-membered aromatic ring system which contains 1 or 2 nitrogen atoms, and wherein the ring system is substituted once or more than once by R₃;

each R₃ independently is halogen; cyano; hydroxy; amino; or —X₃-R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is pyrid-2-yl being substituted in the 4-position and/or in the 6-position by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one class of compounds of the invention, A is pyrid-2-yl being substituted in the 4-position and/or in the 6-position independently by cyano, C₁₋₄alkyl or C₁₋₄alkyloxy.

In one class of compounds of the invention, A is pyrid-2-yl being substituted in the 4-position and/or in the 6-position independently by methyl or methoxy.

In one class of compounds of the invention, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one class of compounds of the invention, A is pyrimidin-2-yl being substituted in the 4-position or in the 4-position and in the 6-position independently by cyano, C₁₋₄alkyl or C₁₋₄alkyloxy.

In one class of compounds of the invention, A is pyrimidin-2-yl being substituted in the 4-position or in the 4-position and in the 6-position independently by methyl or methoxy.

In one class of compounds of the invention, A is a five-membered aromatic ring system which contains from 1 to 4 hetero atoms selected from nitrogen, oxygen and sulfur, wherein the ring system is substituted once or more than once by R₃, and wherein a substituent on a nitrogen in a heterocyclic ring system may not be halogen;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is a five-membered aromatic ring system which contains from 1 to 4 hetero atoms selected from nitrogen, oxygen and sulfur, wherein the ring system is substituted once or more than once by R₃, and wherein a substituent on a nitrogen in a heterocyclic ring system may not be halogen;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₆alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one class of compounds of the invention, A is a five-membered aromatic ring system which contains from 1 to 4 hetero atoms selected from nitrogen, oxygen and sulfur, wherein the ring system is substituted once or more than once independently by C₁₋₄alkyl or C₁₋₄alkyloxy.

In one class of compounds of the invention, A is phenyl being substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl.

In one class of compounds of the invention, A is phenyl being substituted once or more than once by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one class of compounds of the invention, m is 0, 1, 2 or 3. In one embodiment of said class, m is 0 or 1, for example m being 0.

In one class of compounds of the invention, n is 0, 1, 2 or 3. In one embodiment of said class, n is 0 or 1, for example n being 0.

In one class of compounds of the invention, m and n are both 0.

In one class of compounds of the invention, each R₁ or R₂ independently is halogen, C₁₋₆alkyl or C₁₋₆halogenalkyl.

In one class of compounds of the invention, —X₁— is —C(O)— and —X₂— is —N(L-B)—.

In one class of compounds of the invention, —X₁— is —N(L-B)— and —X₂— is —C(O)—.

In one class of compounds of the invention, each R₆ independently is hydrogen, C₁₋₆alkyl or C₁₋₆halogenalkyl. In one embodiment of said class, each R₆ is hydrogen.

In one class of compounds of the invention, B is an eight- to ten-membered fused bicyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen.

In one class of compounds of the invention, B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen.

In one class of compounds of the invention, B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and

each R₇ independently is cyano, C₁₋₆alkyl, C₁₋₆halogenalkyl, halogen, C₁₋₈alkoxy, C₁₋₄alkoxyC₁₋₆alkyl, C₁₋₄alkoxycarbonyl, or a three- to seven-membered monocyclic ring system which is saturated and which may contain from 1 to 2 hetero atoms selected from nitrogen and oxygen, and wherein each ring system may in turn be substituted once or more than once by C₁₋₄alkyl, C₁₋₄alkoxyC₁₋₆alkyl or halogen, and wherein a substituent on a nitrogen in a heterocyclic ring system may not be halogen.

In one class of compounds of the invention, B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and

each R₇ independently is cyano, C₁₋₆alkyl, C₁₋₆halogenalkyl, halogen, C₁₋₆alkoxy, or a three-to seven-membered monocyclic ring system which is saturated and which may contain from 1 to 2 hetero atoms selected from nitrogen and oxygen, and wherein each ring system may in turn be substituted once or more than once by C₁₋₄alkyl or halogen, and wherein a substituent on a nitrogen in a heterocyclic ring system may not be halogen.

In one class of compounds of the invention, B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and

each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one class of compounds of the invention, B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and

each R₇ independently is C₁₋₆alkyl, C₁₋₆alkoxy, C₁₋₆halogenalkyl or halogen.

In one class of compounds of the invention, B is indolyl which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one class of compounds of the invention, B is indolyl which may be substituted once or more than once by R₇, wherein a substituent on the nitrogen of the indolyl may not be halogen; and each R₇ independently is Cl₁₋₆alkyl, C₁₋₆alkoxy, C₁₋₆halogenalkyl or halogen.

In one class of compounds of the invention, B is indol-3-yl which may be substituted once or more than once by R₇, wherein a substituent on the nitrogen of the indol-3-yl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one class of compounds of the invention, B is indol-3-yl which may be substituted once or more than once by R₇, wherein a substituent on the nitrogen of the indol-3-yl may not be halogen; and each R₇ independently is C₁₋₆alkyl, C₁₋₆alkoxy, C₁₋₆halogenalkyl or halogen.

In one class of compounds of the invention, B is indol-4-yl which may be substituted once or more than once by R₇, wherein a substituent on the nitrogen of the indol-3-yl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one class of compounds of the invention, B is indol-4-yl which may be substituted once or more than once by R₇, wherein a substituent on the nitrogen of the indol-3-yl may not be halogen; and each R₇ independently is C₁₋₆alkyl, C₁₋₆alkoxy, C₁₋₆halogenalkyl or halogen.

In one class of compounds of the invention, B is a five- to six-membered monocyclic aromatic ring system which may contain from 1 to 4 hetero atoms selected from nitrogen, oxygen and sulfur, and wherein the ring system may be substituted once or more than once by R₇, and wherein a substituent on a nitrogen in a heterocyclic ring system may not be halogen.

In one class of compounds of the invention, B is a six-membered monocyclic aromatic ring system which may contain 1 to 2 nitrogen atoms, wherein the ring system is substituted once by R₇ₐ, and wherein the ring system may be further substituted once or more than once by R_{7b};

R₇ₐ is a five- to six-membered monocyclic aromatic ring system, which may contain from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a six-membered monocyclic aromatic ring system which may contain 1 to 2 nitrogen atoms, wherein the ring system is substituted once by R₇ₐ, and wherein the ring system may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is phenyl which is substituted once by R₇ₐ, and which may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is phenyl being substituted by R₇ₐ in the ortho-position relative to group L, and wherein said phenyl may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is phenyl being substituted by R₇ₐ in the ortho-position relative to group L; and R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen.

In one class of compounds of the invention, B is phenyl being substituted by R₇ₐ in the meta-position relative to group L, and wherein said phenyl may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is phenyl being substituted by R₇ₐ in the meta-position relative to group L; and R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen.

In one class of compounds of the invention, B is a six-membered monocyclic aromatic ring system which contains 1 to 2 nitrogen atoms, wherein the ring system is substituted once by R₇ₐ, and wherein the ring system may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a six-membered monocyclic aromatic ring system which contains 1 to 2 nitrogen atoms, wherein the ring system is substituted once by R₇ₐ; R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen.

In one class of compounds of the invention, B is a five-membered monocyclic aromatic ring system which contains from 1 to 4 hetero atoms selected from nitrogen, oxygen and sulfur, and wherein the ring system may be substituted once or more than once by R₇, and wherein a substituent on a nitrogen in a heterocyclic ring system may not be halogen.

In one class of compounds of the invention, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is a six-membered monocyclic aromatic ring system, which may contain from 1 to 2 nitrogen atoms, and wherein the ring system may in turn be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is a six-membered monocyclic aromatic ring system, which may contain from 1 to 2 nitrogen atoms, and wherein the ring system may in turn be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In one class of compounds of the invention, B is a five-membered monocyclic aromatic ring system which contains from 1 to 4 hetero atoms selected from nitrogen, oxygen and sulfur, and wherein the ring system is substituted once by phenyl, which may in turn be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano.

In one class of compounds of the invention, B is a five-membered monocyclic aromatic ring system which contains from 1 to 4 hetero atoms selected from nitrogen, oxygen and sulfur, and wherein the ring system is substituted once by phenyl, which may in turn be substituted once or more than once by C₁₋₆alkyl, C₁₋₆halogenalkyl, C₁₋₆alkoxy, C₁₋₆halogenalkoxy, halogen or cyano.

In one class of compounds of the invention, B is a five- to six-membered monocyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;

each R₇ independently is C₁₋₆alkyl, C₁₋₆halogenalkyl, C₁₋₆alkoxy, C₁₋₆halogenalkoxy, C₁₋₄alkoxyC₁₋₆alkyl, halogen, cyano or a three- to seven-membered monocyclic ring system which may be saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo.

In one class of compounds of the invention, B is a five- to six-membered monocyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;

each R₇ independently is C₁₋₆alkyl, C₁₋₆halogenalkyl, C₁₋₆alkoxy, C₁₋₆halogenalkoxy, halogen, cyano or a three- to seven-membered monocyclic ring system which may be saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo.

In one class of compounds of the invention, B is a five- to six-membered monocyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen; each R₇ independently is C₁₋₆alkyl, C₁₋₆halogenalkyl, C₁₋₆alkoxy, C₁₋₆halogenalkoxy, C₁₋₄alkoxyC₁₋₆alkyl, halogen, cyano.

In one class of compounds of the invention, B is a five- to six-membered monocyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen; each R₇ independently is C₁₋₆alkyl, C₁₋₆halogenalkyl, C₁₋₆alkoxy, C₁₋₆halogenalkoxy, halogen, cyano.

In one class of compounds of the invention, B is a five-membered monocyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; each R₇ independently is C₁₋₆alkyl, C₁₋₆halogenalkyl, C₁₋₆alkoxy, C₁₋₆halogenalkoxy, halogen, cyano.

In one class of compounds of the invention, B is a six-membered monocyclic aromatic ring system which contains 1 or 2 nitrogen atoms, wherein the ring system may be substituted once or more than once by R₇; each R₇ independently is C₁₋₆alkyl, C₁₋₆halogenalkyl, C₁₋₆alkoxy, C₁₋₆halogenalkoxy, C₁₋₄alkoxyC₁₋₆alkyl, halogen, cyano.

In one class of compounds of the invention, B is a six-membered monocyclic aromatic ring system which contains 1 or 2 nitrogen atoms, wherein the ring system may be substituted once or more than once by R₇; each R₇ independently is C₁₋₆alkyl, C₁₋₆halogenalkyl, C₁₋₆alkoxy, C₁₋₆halogenalkoxy, halogen, cyano.

In one class of compounds of the invention, B is phenyl which may be substituted once or more than once by R₇; each R₇ independently is C₁₋₆alkyl, C₁₋₆halogenalkyl, C₁₋₆alkoxy, C₁₋₆halogenalkoxy, C₁₋₄alkoxyC₁₋₆alkyl, halogen, cyano.

In one class of compounds of the invention, B is phenyl which may be substituted once or more than once by R₇; each R₇ independently is C₁₋₆alkyl, C₁₋₆halogenalkyl, C₁₋₆alkoxy, C₁₋₆halogenalkoxy, halogen, cyano.

In embodiment E1.1 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —C(O)— and —X₂— is —N(CH₂—B)—;

and B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and each R₇ independently is C₁₋₆alkyl, C₁₋₆halogenalkyl or halogen.

In one embodiment within said embodiment E1.1, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E1.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E1.1, B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E1.1, B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E1.1, B is indol-3-yl which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E1.1, B is indol-3-yl which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E1.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E1.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E1.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-3-yl, which may be substituted once or more than once by R₇, wherein a substituent on the nitrogen of the indolyl may not be halogen; and each R₇ independently is C₁₋₆alkyl, C₁₋₆halogenalkyl, C₁₋₄alkoxyC₁₋₆alkyl, C₁₋₄alkoxycarbonyl, or halogen.

In one embodiment within said embodiment E1.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-3-yl, which may be substituted once or more than once by R₇, wherein a substituent on the nitrogen of the indolyl may not be halogen; and each R₇ independently is C₁₋₆alkoxy, C₁₋₆halogenalkyl or halogen.

In one embodiment within said embodiment E1.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E1.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E1.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is indol-3-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E1.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-3-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In embodiment E1.2 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the Cₘcycloalkyl in turn may be substituted by halogen or C₁₋₄alkyl;
m and n both are 0;

### —X₁— is —C(O)— and —X₂— is —N(CH₂—B)—;

and B is phenyl being substituted by R₇ₐ in the ortho-position relative to group L, and wherein said phenyl may be further substituted once or more than once by R_{7b});
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.2, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₇₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E1.2, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In embodiment E1.3 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —C(O)— and —X₂— is —N(CH₂—B)—;

B is phenyl being substituted by R₇ₐ in the meta-position relative to group L, and wherein said phenyl may be further substituted once or more than once by R_{7b};
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.3, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E1.3, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In embodiment E1.4 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —C(O)— and —X₂— is —N(CH₂—B)—;

B is a six-membered monocyclic aromatic ring system which contains 1 to 2 nitrogen atoms, wherein the ring system is substituted once by R₇ₐ, and wherein the ring system may be further substituted once or more than once by R_{7b};
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.4, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₃₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₃₋₄alkyl; or C₃₋₄cycloalkyl.

In one embodiment within said embodiment E1.4, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In embodiment E1.5 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₃₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —C(O)— and —X₂— is —N(CH₂—B)—;

and B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;
R₇ₐ is a six-membered monocyclic aromatic ring system, which may contain from 1 to 2 nitrogen atoms, and wherein the ring system may in turn be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.5, A is pyrid-2-yl being substituted in the 4-position and/or in the 6-position by R₃,

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; 4alkylamino-C₁₋₄alkyl; di(C₁₋₄alkyl)amino-C₁₋₄alkyl; or C₃₋₄cycloalkyl.

In one embodiment within said embodiment E1.5, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₇₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E1.5, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.5, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.5,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C_{1-g}alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;
R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.5, A is pyrimidin-2-yl being substituted in the 4-position or in the 4-position and in the 6-position by R₃;

each R₃ independently is halogen; cyano; hydroxy; amino; or —X₃-R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;
R₇ₐ is phenyl, which may be substituted once or more than once by C₁₋₆alkyl, C₁₋₆halogenalkyl, C₁₋₆alkoxy, C₁₋₆halogenalkoxy, C₁₋₄alkoxyC₁₋₆alkyl, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E1.5, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;
R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In embodiment E2.1 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —N(CH₂—B)— and —X₂— is —C(O)—;

and B is a nine-membered fused bicyclic aromatic ring system which contains from 1 to 4 hetero atoms selected from nitrogen, oxygen and sulfur, wherein the ring system may be substituted once or more than once by R₇, and wherein a substituent on a nitrogen in a heterocyclic ring system may not be halogen; and each R₇ independently is C₁₋₆alkyl, C₁₋₆halogenalkyl or halogen.

In one embodiment within said embodiment E2.1, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.1, B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1, B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E2.1, B is indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1, B is indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E2.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1,

A is pyrid-2-yl being substituted in the 4-position and/or in the 6-position by R₃,
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E2.1,

A is pyrid-2-yl being substituted in the 4-position and/or in the 6-position by R₃,
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₆alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl: C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-4-yl, which may be substituted once or more than once by R₇, wherein a substituent on the nitrogen of the indolyl may not be halogen; and each R₇ independently is C₁₋₆alkoxy, C₁₋₆halogenalkyl or halogen.

In one embodiment within said embodiment E2.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl; and
B is indolyl, e.g. indol-3-yl or indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In one embodiment within said embodiment E2.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl, C₁₋₄alkoxyC₁₋₆alkyl, C₁₋₄alkoxycarbonyl, or halogen.

In one embodiment within said embodiment E2.1, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is indol-4-yl, which may be substituted once or more than once by R₇, wherein a substituent on the nitrogen of the indolyl may not be halogen; and each R₇ independently is C₁₋₆alkyl, C₁₋₆alkoxy, C₁₋₆halogenalkyl or halogen.

In embodiment E2.2 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —N(CH₂—B)— and —X₂— is —C(O)—;

and B is phenyl being substituted by R₇ₐ in the ortho-position relative to group L, and wherein said phenyl may be further substituted once or more than once by R_{7b};
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.2, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.2, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In embodiment E2.3 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the Cₘcycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —N(CH₂—B)— and —X₂— is —C(O)—;

B is phenyl being substituted by R₇ₐ in the meta-position relative to group L, and wherein said phenyl may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and

each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.3, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.3, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In embodiment E2.4 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —N(CH₂—B)— and —X₂— is —C(O)—;

B is a six-membered monocyclic aromatic ring system which contains 1 to 2 nitrogen atoms,
wherein the ring system is substituted once by R₇ₐ, and wherein the ring system may be further substituted once or more than once by R_{7b};
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.4, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.4, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In embodiment E2.5 of the invention, a compound of the formula I is provided wherein A is a six-membered aromatic ring system which contains 1 or 2 nitrogen atoms, and wherein the ring system is substituted once or more than once by R₃;

each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—; and
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m and n both are 0;

### —X₁— is —N(CH₂—B)— and —X₂— is —C(O)—;

and B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is a six-membered monocyclic aromatic ring system, which may contain from 1 to 2 nitrogen atoms, and wherein the ring system may in turn be substituted once or more than once by C₁₋₆alkyl, C₁₋₆halogenalkyl, C₁₋₆alkoxy, C₁₋₆halogenalkoxy, halogen or cyano; and

each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.5, A is pyrid-2-yl being substituted in the 4-position and/or in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.5, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄aminoalkyl; C₁₋₄alkylamino-C₁₋₄alkyl; di(C₁₋₄alkyl)amino-C₁₋₄alkyl; or C₃₋₄cycloalkyl.

In one embodiment within said embodiment E2.5, B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and
each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.5,

A is pyrid-2-yl being substituted in the 4-position and/or in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl; and
B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and

each R_{7b} independently is C₁₋₆alkyl, C₁₋₆halogenalkyl, C₁₋₆alkoxy, C₁₋₆halogenalkoxy, halogen or cyano.

In one embodiment within said embodiment E2.5, A is pyrimidin-2-yl being substituted in the 4-position or in the 4-position and in the 6-position by R₃;

each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-Cₘalkyl; di(C₁₋₄alkyl)amino-C₁₋₄alkyl; or C₃₋₄cycloalkyl; and
B is a five-membered monocyclic aromatic ring system which contains from 1 to 4 hetero atoms selected from nitrogen, oxygen and sulfur, and wherein the ring system is substituted once by R₇ₐ, and wherein the ring system may be further substituted once or more than once by R_{7b}, and wherein a substituent on a nitrogen in a heterocyclic ring system may not be halogen;

R₇ₐ is phenyl, which may be substituted once or more than once by C₁₋₆alkyl, C₁₋₆halogenalkyl, C₁₋₆alkoxy, C₁₋₆halogenalkoxy, halogen or cyano; and

each R_{7b} independently is C₁₋₆alkyl, C₁₋₆halogenalkyl, C₁₋₆alkoxy, C₁₋₆halogenalkoxy, halogen or cyano.

In one embodiment, the invention provides a compound selected from
9-(4,6-dimethylpyrimidin-2-yl)-2-(2-(3-methyl-1,2,4-oxadiazol-5-yl)benzyl)-2,9-diazaspiro[5.5]undecan-1-one;
2-(2-(2H-1,2,3-triazol-2-yl)benzyl)-9-(4,6-dimethylpyrimidin-2-yl)-2,9-diazaspiro[5.5]undecan-1-one;
1-((1H-indol-4-yl)methyl)-9-(4,6-dimethylpyrimidin-2-yl)-1,9-diazaspiro[5.5]undecan-2-one;
2-((1H-indol-3-yl)methyl)-9-(4-methoxy-6-methylpyrimidin-2-yl)-2,9-diazaspiro[5.5]undecan-1-one;
2-((1H-indol-3-yl)methyl)-9-(4,6-dimethylpyrimidin-2-yl)-2,9-diazaspiro[5.5]undecan-1-one;
9-(4,6-dimethylpyrimidin-2-yl)-2-((1-methyl-1H-indol-3-yl)methyl)-2,9-diazaspiro[5.5]undecan-1-one;
1-((1H-indol-3-yl)methyl)-9-(6-methylpyrazin-2-yl)-1,9-diazaspiro[5.5]undecan-2-one;
2-((1H-pyrrolo[2,3-b]pyridin-3-yl)methyl)-9-(6-methoxypyrazin-2-yl)-2,9-diazaspiro[5.5]undecan-1-one;
2-((1H-indol-3-yl)methyl)-9-(6-methylpyridin-2-yl)-2,9-diazaspiro[5.5]undecan-1-one;
2-(2-((1H-indol-3-yl)methyl)-1-oxo-2,9-diazaspiro[5.5]undecan-9-yl)isonicotinonitrile;
2-((1H-indol-3-yl)methyl)-9-(6-methylpyrazin-2-yl)-2,9-diazaspiro[5.5]undecan-1-one;
2-((1H-indol-3-yl)methyl)-9-(4-methylpyrimidin-2-yl)-2,9-diazaspiro[5.5]undecan-1-one;
2-((1H-indol-3-yl)methyl)-9-(4-methoxypyrimidin-2-yl)-2,9-diazaspiro[5.5]undecan-1-one;
6-(1-((1H-indol-4-yl)methyl)-2-oxo-1,9-diazaspiro[5.5]undecan-9-yl)nicotinonitrile;
9-(4,6-dimethylpyrimidin-2-yl)-2-(2-(furan-2-yl)benzyl)-2,9-diazaspiro[5.5]undecan-1-one;
1-(2-(2H-1,2,3-triazol-2-yl)benzyl)-9-(4-methylpyrimidin-2-yl)-1,9-diazaspiro[5.5]undecan-2-one;
2-((1H-indol-3-yl)methyl)-9-(5-methyl-1,3,4-oxadiazol-2-yl)-2,9-diazaspiro[5.5]undecan-1-one;
2-((1H-pyrrolo[2,3-b]pyridin-3-yl)methyl)-9-(6-chloropyrazin-2-yl)-2,9-diazaspiro[5.5]undecan-1-one;
2-((1H-pyrrolo[2,3-b]pyridin-3-yl)methyl)-9-(6-methylpyrazin-2-yl)-2,9-diazaspiro[5.5]undecan-1-one;
2-((2,3-dihydrobenzo[b][1,4]dioxin-5-yl)methyl)-9-(4,6-dimethylpyrimidin-2-yl)-2,9-diazaspiro[5.5]undecan-1-one;
9-(6-chloropyrazin-2-yl)-2-((2,3-dihydrobenzo[b][1,4]dioxin-5-yl)methyl)-2,9-diazaspiro[5.5]undecan-1-one;
9-(4,6-dimethylpyrimidin-2-yl)-2-(3-(5-methyl-1,2,4-oxadiazol-3-yl)benzyl)-2,9-diazaspiro[5.5]undecan-1-one;
9-(4,6-dimethylpyrimidin-2-yl)-2-((5-methyl-3-phenylisoxazol-4-yl)methyl)-2,9-diazaspiro[5.5]undecan-1-one;
2-(2-(2H-1,2,3-triazol-2-yl)benzyl)-9-(4-methylpyrimidin-2-yl)-2,9-diazaspiro[5.5]undecan-1-one;
2-(2-(2-(2H-1,2,3-triazol-2-yl)benzyl)-1-oxo-2,9-diazaspiro[5.5]undecan-9-yl)-6-methylpyrimidine-4-carbonitrile;
6-(2-(2-(2H-1,2,3-triazol-2-yl)benzyl)-1-oxo-2,9-diazaspiro[5.5]undecan-9-yl)picolinonitrile;
2-(2-(2-(2H-1,2,3-triazol-2-yl)benzyl)-1-oxo-2,9-diazaspiro[5.5]undecan-9-yl)isonicotinonitrile;
2-methyl-6-(1-oxo-2-(3-(pyrimidin-2-yl)benzyl)-2,9-diazaspiro[5.5]undecan-9-yl)isonicotinonitrile;
2-methyl-6-(2-(3-(5-methyloxazol-2-yl)benzyl)-1-oxo-2,9-diazaspiro[5.5]undecan-9-yl)isonicotinonitrile;
9-(4-methylpyrimidin-2-yl)-2-(3-(pyrimidin-2-yl)benzyl)-2,9-diazaspiro[5.5]undecan-1-one;
9-(4,6-dimethylpyrimidin-2-yl)-2-(3-(pyrimidin-2-yl)benzyl)-2,9-diazaspiro[5.5]undecan-1-one;
6-methyl-2-(1-oxo-2-(3-(pyrimidin-2-yl)benzyl)-2,9-diazaspiro[5.5]undecan-9-yl)pyrimidine-4-carbonitrile;
2-(1-oxo-2-(3-(pyrimidin-2-yl)benzyl)-2,9-diazaspiro[5.5]undecan-9-yl)pyrimidine-4-carbonitrile;
6-(1-oxo-2-(3-(pyrimidin-2-yl)benzyl)-2,9-diazaspiro[5.5]undecan-9-yl)picolinonitrile;
2-(1-oxo-2-(3-(pyrimidin-2-yl)benzyl)-2,9-diazaspiro[5.5]undecan-9-yl)isonicotinonitrile;
9-(4,6-dimethylpyrimidin-2-yl)-2-(3-(5-methyloxazol-2-yl)benzyl)-2,9-diazaspiro[5.5]undecan-1-one;
6-methyl-2-(2-(3-(5-methyloxazol-2-yl)benzyl)-1-oxo-2,9-diazaspiro[5.5]undecan-9-yl)pyrimidine-4-carbonitrile;
2-(3-(5-methyloxazol-2-yl)benzyl)-9-(4-methylpyrimidin-2-yl)-2,9-diazaspiro[5.5]undecan-1-one;
6-(2-(3-(5-methyloxazol-2-yl)benzyl)-1-oxo-2,9-diazaspiro[5.5]undecan-9-yl)picolinonitrile;
2-(2-(3-(5-methyloxazol-2-yl)benzyl)-1-oxo-2,9-diazaspiro[5.5]undecan-9-yl)isonicotinonitrile;
1-((1H-indol-4-yl)methyl)-9-(6-methylpyrazin-2-yl)-1,9-diazaspiro[5.5]undecan-2-one;
1-((1H-indol-4-yl)methyl)-9-(4-methylpyrimidin-2-yl)-1,9-diazaspiro[5.5]undecan-2-one;
1-((1H-indol-4-yl)methyl)-9-(6-methoxypyrazin-2-yl)-1,9-diazaspiro[5.5]undecan-2-one;
1-((1H-indol-4-yl)methyl)-9-(6-chloropyrazin-2-yl)-1,9-diazaspiro[5.5]undecan-2-one;
2-((1H-indazol-3-yl)methyl)-9-(4,6-dimethylpyrimidin-2-yl)-2,9-diazaspiro[5.5]undecan-1-one;
2-((1H-indol-3-yl)methyl)-9-(4-(dimethylamino)pyrimidin-2-yl)-2,9-diazaspiro[5.5]undecan-1-one;
2-((1H-indol-3-yl)methyl)-9-(2-methoxypyrimidin-4-yl)-2,9-diazaspiro[5.5]undecan-1-one;
2-((1H-indol-3-yl)methyl)-9-(2-chloropyrimidin-4-yl)-2,9-diazaspiro[5.5]undecan-1-one;
2-((1H-indol-3-yl)methyl)-9-(4-(trifluoromethyl)pyrimidin-2-yl)-2,9-diazaspiro[5.5]undecan-1-one;
2-((1H-indol-3-yl)methyl)-9-(4-isopropylpyrimidin-2-yl)-2,9-diazaspiro[5.5]undecan-1-one;
2-((1H-indol-3-yl)methyl)-9-(4-chloropyrimidin-2-yl)-2,9-diazaspiro[5.9]undecan-1-one;
2-((1H-indol-3-yl)methyl)-9-(4-ethoxypyrimidin-2-yl)-2,9-diazaspiro[5.5]undecan-1-one;
9-(6-chloropyrazin-2-yl)-2-((1-methyl-1H-indol-3-yl)methyl)-2,9-diazaspiro[5.5]undecan-1-one;
9-(4,6-dimethylpyrimidin-2-yl)-2-((1-ethyl-1H-indol-3-yl)methyl)-2,9-diazaspiro[5.5]undecan-1-one;
1-((1H-indol-3-yl)methyl)-9-(6-methoxypyrazin-2-yl)-1,9-diazaspiro[5.5]undecan-2-one;
2-((2,3-dihydrobenzo[b][1,4]dioxin-5-yl)methyl)-9-(6-methoxypyrazin-2-yl)-2,9-diazaspiro[5.5]undecan-1-one;
2-((1H-indol-3-yl)methyl)-9-m-tolyl-2,9-diazaspiro[5.5]undecan-1-one;
2-((1H-indol-3-yl)methyl)-9-(5-methylpyridin-3-yl)-2,9-diazaspiro[5.5]undecan-1-one;
2-((1H-indol-3-yl)methyl)-9-(3,4-dimethoxyphenyl)-2,9-diazaspiro[5.5]undecan-1-one;
2-((1H-indol-3-yl)methyl)-9-(3-methoxyphenyl)-2,9-diazaspiro[5.5]undecan-1-one;
2-((1H-indol-3-yl)methyl)-9-(2-methoxypyridin-4-yl)-2,9-diazaspiro[5.5]undecan-1-one;
2-((1H-indol-3-yl)methyl)-9-(5-methoxypyridin-3-yl)-2,9-diazaspiro[5.5]undecan-1-one;
2-((1H-indol-3-yl)methyl)-9-(2,6-dimethylpyridin-4-yl)-2,9-diazaspiro[5.5]undecan-1-one;
2-((1H-indol-3-yl)methyl)-9-(5,6-dimethylpyridin-2-yl)-2,9-diazaspiro[5.5]undecan-1-one;
2-((1H-indol-3-yl)methyl)-9-(6-methoxypyridin-2-yl)-2,9-diazaspiro[5.5]undecan-1-one;
2-((1H-indol-3-yl)methyl)-9-(3-methylpyridin-2-yl)-2,9-diazaspiro[5.5]undecan-1-one;
2-((1H-indol-3-yl)methyl)-9-(4-methylpyridin-2-yl)-2,9-diazaspiro[5.5]undecan-1-one;
2-((1H-indol-3-yl)methyl)-9-(5-methylpyridin-2-yl)-2,9-diazaspiro[5.5]undecan-1-one;
6-(2-((1H-indol-3-yl)methyl)-1-oxo-2,9-diazaspiro[5.5]undecan-9-yl)nicotinonitrile;
2-((1H-indol-3-yl)methyl)-9-(2-methylpyrimidin-4-yl)-2,9-diazaspiro[5.5]undecan-1-one;
2-((1H-indol-3-yl)methyl)-9-(2,6-dimethylpyrimidin-4-yl)-2,9-diazaspiro[5.5]undecan-1-one;
2-((1H-indol-3-yl)methyl)-9-(5-methylpyrazin-2-yl)-2,9-diazaspiro[5.5]undecan-1-one;
2-((1H-indol-3-yl)methyl)-9-(6-(methoxymethyl)pyridin-2-yl)-2,9-diazaspiro[5.5]undecan-1-one;
9-(4,6-dimethylpyridin-2-yl)-2-(3-(pyrimidin-2-yl)benzyl)-2,9-diazaspiro[5.5]undecan-1-one;
2-(2-((1H-indol-3-yl)methyl)-1-oxo-2,9-diazaspiro[5.5]undecan-9-yl)-6-methylpyrimidine-4-carbonitrile;
2-(2-((1H-indol-3-yl)methyl)-1-oxo-2,9-diazaspiro[5.5]undecan-9-yl)pyrimidine-4,6-dicarbonitrile;
6-(2-((1H-indol-3-yl)methyl)-1-oxo-2,9-diazaspiro[5.5]undecan-9-yl)pyrazine-2-carbonitrile;
2-(2-((1H-indol-3-yl)methyl)-1-oxo-2,9-diazaspiro[5.5]undecan-9-yl)-6-methylisonicotinonitrile;
2-((1H-indol-3-yl)methyl)-9-(4-ethylpyrimidin-2-yl)-2,9-diazaspiro[5.5]undecan-1-one;
2-(2-((1H-indol-3-yl)methyl)-1-oxo-2,9-diazaspiro[5.5]undecan-9-yl)pyrimidine-4-carbonitrile;
2-((1H-indol-3-yl)methyl)-9-(4,5-dimethylpyrimidin-2-yl)-2,9-diazaspiro[5.5]undecan-1-one;
2-((1H-indol-3-yl)methyl)-9-(6-methoxypyrazin-2-yl)-2,9-diazaspiro[5.5]undecan-1-one;
6-(2-((1H-indol-3-yl)methyl)-1-oxo-2,9-diazaspiro[5.5]undecan-9-yl)picolinonitrile;
2-((1H-indol-3-yl)methyl)-9-(6-hydroxypyridin-2-yl)-2,9-diazaspiro[5.5]undecan-1-one;
2-((1H-indol-3-yl)methyl)-9-(5-methyl-1,3,4-thiadiazol-2-yl)-2,9-diazaspiro[5.5]undecan-1-one;
2-((1H-indol-3-yl)methyl)-9-(3,6-dimethylpyrazin-2-yl)-2,9-diazaspiro[5.5]undecan-1-one;
2-((1H-indol-3-yl)methyl)-9-(6-methoxypyrimidin-4-yl)-2,9-diazaspiro[5.5]undecan-1-one;
2-((1H-indol-3-yl)methyl)-9-(4,6-dimethoxypyrimidin-2-yl)-2,9-diazaspiro[5.5]undecan-1-one;
2-((1H-indol-3-yl)methyl)-9-(5-(trifluoromethyl)pyridin-2-yl)-2,9-diazaspiro[5.5]undecan-1-one;
9-(4-methylpyrimidin-2-yl)-1-(4-(pyrimidin-2-yl)benzyl)-1,9-diazaspiro[5.5]undecan-2-one;
2-(1-((1H-indol-4-yl)methyl)-2-oxo-1,9-diazaspiro[5.5]undecan-9-yl)isonicotinonitrile;
6-(1-((1H-indol-4-yl)methyl)-2-oxo-1,9-diazaspiro[5.5]undecan-9-yl)picolinonitrile;
9-(4,6-dimethylpyrimidin-2-yl)-2-(2-(oxazol-2-yl)benzyl)-2,9-diazaspiro[5.5]undecan-1-one;
1-(3-(2H-1,2,3-triazol-2-yl)benzyl)-9-(4-methylpyrimidin-2-yl)-1,9-diazaspiro[5.5]undecan-2-one;
9-(4-methylpyrimidin-2-yl)-1-((5-phenyl-1,3,4-oxadiazol-2-yl)methyl)-1,9-diazaspiro[5.5]undecan-2-one;
1-((2-methyl-1H-indol-4-yl)methyl)-9-(4-methylpyrimidin-2-yl)-1,9-diazaspiro[5.5]undecan-2-one;
1-(3-(3-methyl-1,2,4-oxadiazol-5-yl)benzyl)-9-(4-methylpyrimidin-2-yl)-1,9-diazaspiro[5.5]undecan-2-one;
9-(4-methylpyrimidin-2-yl)-1-((6-(pyrrolidin-1-yl)pyridin-2-yl)methyl)-1,9-diazaspiro[5.5]undecan-2-one;
1-(2-(3-methyl-1,2,4-oxadiazol-5-yl)benzyl)-9-(4-methylpyrimidin-2-yl)-1,9-diazaspiro[5.5]undecan-2-one;
2-((1H-indol-3-yl)methyl)-9-(2-methylpyridin-4-yl)-2,9-diazaspiro[5.5]undecan-1-one;
4-fluoro-3-((9-(4-methylpyrimidin-2-yl)-2-oxo-1,9-diazaspiro[5.5]undecan-1-yl)methyl)benzonitrile;
2-((1H-indol-3-yl)methyl)-9-(5-fluoro-4-methylpyrimidin-2-yl)-2,9-diazaspiro[5.5]undecan-1 one;
9-(4,6-dimethylpyrimidin-2-yl)-2-((7-methyl-1H-indol-3-yl)methyl)-2,9-diazaspiro[5.5]undecan-1-one;
9-(4,6-dimethylpyrimidin-2-yl)-2-((5-methyl-1H-indol-3-yl)methyl)-2,9-diazaspiro[5.5]undecan-1-one;
3-((9-(4,6-dimethylpyrimidin-2-yl)-1-oxo-2,9-diazaspiro[5.5]undecan-2-yl)methyl)-1H-indole-5-carbonitrile;
9-(4,6-dimethylpyrimidin-2-yl)-2-((4-phenyl-1H-pyrazol-3-yl)methyl)-2,9-diazaspiro[5.5]undecan-1-one;
9-(4,6-dimethylpyrimidin-2-yl)-2-((1-methyl-4-phenyl-1H-pyrazol-3-yl)methyl)-2,9-diazaspiro[5.5]undecan-1-one;
9-(4,6-dimethylpyrimidin-2-yl)-2-((2-methyl-5-phenyloxazol-4-yl)methyl)-2,9-diazaspiro[5.5]undecan-1-one;
9-(4,6-dimethylpyrimidin-2-yl)-2-((2-methyl-5-phenylthiazol-4-yl)methyl)-2,9-diazaspiro[5.5]undecan-1-one;
9-(4,6-dimethylpyrimidin-2-yl)-2-((5-(3-methoxyphenyl)-2-methylthiazol-4-yl)methyl)-2,9-diazaspiro[5.5]undecan-1-one;
2-((1H-indol-3-yl)methyl)-9-(4-ethyl-6-methylpyrimidin-2-yl)-2,9-diazaspiro[5.5]undecan-1-one;
2-((1H-indazol-3-yl)methyl)-9-(4-methoxy-6-methylpyrimidin-2-yl)-2,9-diazaspiro[5.5]undecan-1-one;
2-((1H-indol-3-yl)methyl)-9-(4-(dimethylamino)-6-methylpyrimidin-2-yl)-2,9-diazaspiro[5.5]undecan-1-one;
and 9-(4-methoxypyrimidin-2-yl)-2-(2-(3-methyl-1,2,4-oxadiazol-5-yl)benzyl)-2,9-diazaspiro[5.5]undecan-1-one;
Methyl 3-((9-(4,6-dimethylpyrimidin-2-yl)-1-oxo-2,9-diazaspiro[5.5]undecan-2-yl)methyl)-1H-indole-5-carboxylate;
9-(4,6-dimethylpyrimidin-2-yl)-2-((5-(methoxymethyl)-1H-indol-3-yl)methyl)-2,9-diazaspiro[5.5]undecan-1-one;
2-((5-(1H-pyrazol-1-yl)-1H-indol-3-yl)methyl)-9-(4,6-dimethylpyrimidin-2-yl)-2,9-diazaspiro[5.5]undecan-1-one;
2-((5-bromo-2-methyl-2H-1,2,3-triazol-4-yl)methyl)-9-(4,6-dimethylpyrimidin-2-yl)-2,9-diazaspiro[5.5]undecan-1-one;
9-(4,6-dimethylpyrimidin-2-yl)-2-((5-(3-(methoxymethyl)phenyl)-2-methyl-2H-1,2,3-triazol-4-yl)methyl)-2,9-diazaspiro[5.5]undecan-1-one;
9-(4,6-dimethylpyrimidin-2-yl)-2-((5-(6-methoxypyridin-2-yl)-2-methyl-2H-1,2,3-triazol-4-yl)methyl)-2,9-diazaspiro[5.5]undecan-1-one;
9-(4,6-dimethylpyrimidin-2-yl)-2-((5-(5-methoxypyridin-3-yl)-2-methyl-2H-1,2,3-triazol-4-yl)methyl)-2,9-diazaspiro[5.5]undecan-1-one;
9-(4,6-dimethylpyrimidin-2-yl)-2-((5-(2-methoxypyridin-4-yl)-2-methyl-2H-1,2,3-triazol-4-yl)methyl)-2,9-diazaspiro[5.5]undecan-1-one;
9-(4,6-dimethylpyrimidin-2-yl)-2-((5-(3-(methoxymethyl)phenyl)oxazol-4-yl)methyl)-2,9-diazaspiro[5.5]undecan-1-one;
9-(4,6-dimethylpyrimidin-2-yl)-2-((5-(3-(methoxymethyl)phenyl)-2-methyloxazol-4-yl)methyl)-2,9-diazaspiro[5.5]undecan-1-one;
9-(4,6-dimethylpyrimidin-2-yl)-2-(3-(methoxymethyl)benzyl)-2,9-diazaspiro[5.5]undecan-1-one;
9-(4,6-dimethylpyrimidin-2-yl)-2-(2-(methoxymethyl)benzyl)-2,9-diazaspiro[5.5]undecan-1-one; and
9-(4-methoxy-6-methylpyrimidin-2-yl)-2-(3-(methoxymethyl)benzyl)-2,9-diazaspiro[5.5]undecan-1-one
   wherein said compound is in free form or in salt form or in pharmaceutically acceptable salt form.

In a further aspect, the invention also provides a process for the production of compounds of the formula I.

Compounds of the formula Ia in free form or in salt form

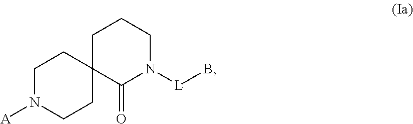

wherein A, L and B are as defined under formula I, are obtainable according to the following processes as described in scheme 1 or scheme 1a:

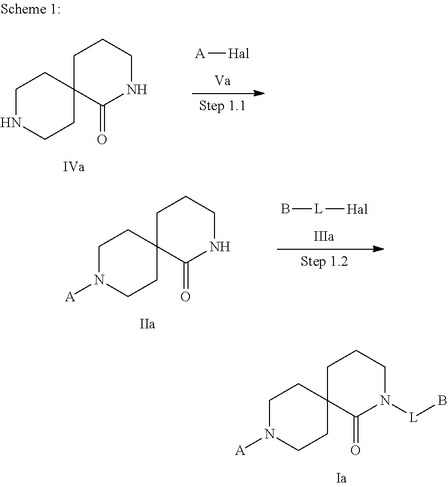

The process steps are described in more detail below:

### Step 1.1:

A compound of formula IIa in free form or in salt form, in which A is as defined under formula I, may be obtained by reacting the compound of formula IVa in free form or in salt form—being 2,9-diaza-spiro[5.5]undecan-1-one—with a compound of formula Va, in which A is defined under formula I and Hal is a halogen atom, such as chloro or bromo, in the presence of a base, such as K₂CO₃, and in the presence of a suitable solvent, such as dimethylformamide.

### Step 1.2:

A compound of formula Ia in free form or in salt form, in which A, L and B are as defined under formula I, may be obtained by reacting the compound of formula IIa in free form or in salt form with a compound of formula IIIa, in which B and L are as defined under formula I and Hal is a halogen atom, such as chloro or bromo, in the presence of a strong base, such as NaH, and in the presence of a suitable solvent, such as tetrahydrofuran.

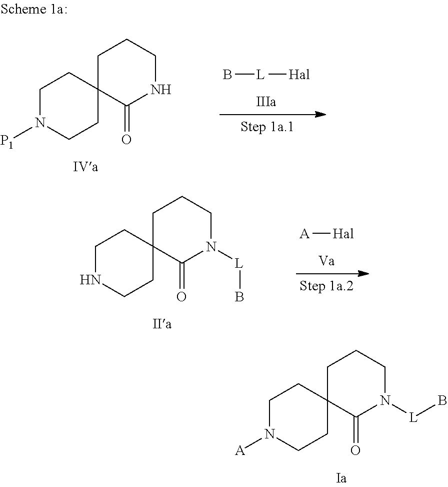

The process steps are described in more detail below:

### Step 1a.1:

A compound of formula II′a in free form or in salt form, in which L and B are as defined under formula I, may be obtained by reacting the compound of formula IV′a in free form or in salt form in which P₁ is a protecting group such as tert-butyl-oxy-carbonyl, with a compound of formula IIIa, in which B and L are as defined under formula I and Hal is a halogen atom, such as chloro or bromo, wherein B may bear a protecting group, such as toluenesulfonyl, in the presence of a base, such as sodium hydride, and a phase transfer reagent such as tetrabutylammonium iodide and in the presence of a suitable solvent, such as tetrahydrofuran, followed by deprotection using a suitable deprotecting agent such as trifluoroacetic acid in a suitable solvent such as isopropyl acetate, and optionally deprotection using a suitable base such as caesium carbonate in a suitable solvent such as methanol.

### Step 1a.2:

A compound of formula Ia in free form or in salt form, in which A, L and B are as defined under formula I, may be obtained by reacting the compound of formula II′a in free form or in salt form with a compound of formula Va, in which A is as defined under formula I and Hal is a halogen atom, such as chloro or bromo, in the presence of a base such as N,N-diisopropylethylamine and a catalyst such as 1,8-diazabicyclo[5.4.0]undec-7-ene (DBU) in a suitable solvent such as acetonitrile under heat and/or pressure.

Compounds of the formula Ib in free form or in salt form,

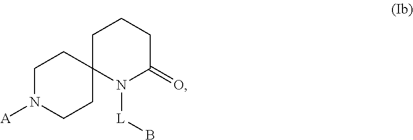

wherein A, L and B are as defined under formula I, are obtainable according to the following process as described in scheme 2:

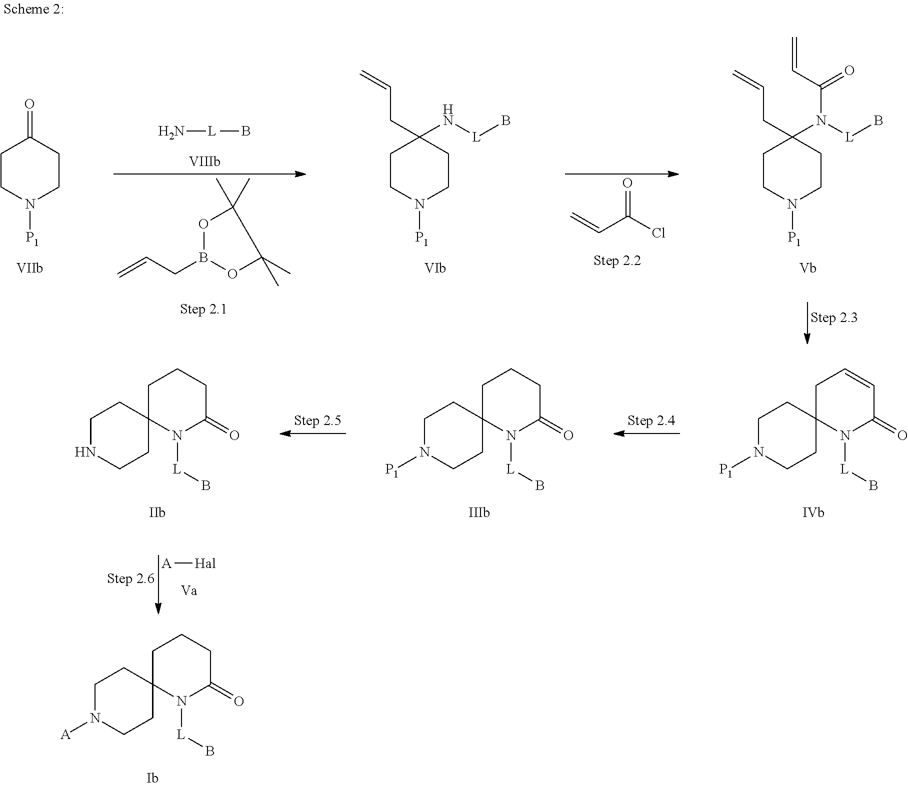

The process steps are described in more detail below:

Step 2.1: A compound of formula VIb, in which L and B are as defined under formula I and P, is a protecting group, such as tertiary-butyl-oxy-carbonyl, may be obtained by reacting a compound of formula VIIb, in which P₁ is a protecting group as defined under formula VIb, with a compound of formula VIIIb, in which L and B are as defined under formula I, and with allyl boronic acid pinacol ester (being depicted in scheme 2), in the presence of a water-binding agent, such as a 4 Å (4 angstroem) molecular sieve, and in the presence of a suitable solvent, such as toluene.

### Step 2.2:

A compound of formula Vb, in which L and B are as defined under formula I and P₁ is a protecting group as defined under formula VIb, may be obtained by reacting the compound of formula VIb with acroloyl chloride (being depicted in scheme 2) in the presence of a base, such as Huenig's base (DIPEA), and in the presence of a suitable solvent, such as dichloromethane.

### Step 2.3:

A compound of formula IVb, in which L and B are as defined under formula I and P₁ is a protecting group as defined under formula VIb, may be obtained by conversion of the compound of formula Vb via ring closure metathesis using a suitable catalyst, such as a Grubbs 2^{nd} generation catalyst, in the presence of a suitable solvent, such as dichloromethane, under an inert gas atmosphere, e.g. under an argon atmosphere.

### Step 2.4:

A compound of formula IIIb, in which L and B are as defined under formula I and P₁ is a protecting group as defined under formula VIb, may be obtained by hydrogenation of the compound of formula IVb using a suitable hydrogenation agent, such as hydrogen and a Pd/C-catalyst, in the presence of a suitable solvent, such as methanol.

### Step 2.5:

A compound of formula fib, in which L and B are as defined under formula I, may be obtained by deprotecting the compound of formula IIIb with a strong acid, such as trifluoroacetic acid, in the presence of a suitable solvent, such as dichloromethane.

### Step 2.6:

A compound of formula Ib, in which A, L and B are as defined under formula I, may be obtained by reacting the compound of formula IIb with the compound of formula Va (said compound being described under scheme 1 above) in the presence of a base, such as K₂CO₃, and in the presence of a suitable solvent, such as dimethylformamide.

Further compounds of formula I in free form or in salt form may be obtainable from compounds of formula Ia or Ib in free form or in salt form—prepared as described according to scheme 1 or scheme 2—by reduction, oxidation and/or other functionalization of resulting compounds and/or by cleavage of any protecting group(s) optionally present, and of recovering the so obtainable compound of the formula I.

The reactions can be effected according to conventional methods, for example as described in the Examples.

The work-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, e.g. as described in the Examples, which processes are further aspects of the invention.

The starting materials of the formulae IIIa, IVa, Va, VIIb and VIIIb are known or may be prepared according to conventional procedures starting from known compounds, for example as described in the Examples. In some cases, an intermediate of scheme 1 or scheme 2 may be known. In such a situation, said intermediate could be used as an alternative starting point for the process according to scheme 1 or scheme 2. All starting materials and intermediates may be used in free form or in salt form.

In a further aspect, the invention also provides a process for the production of compounds of the formula Ia in free form or in salt form

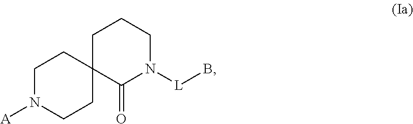

in which A, B and L are as defined under formula I, which comprises reacting a compound of the formula IIa in free form or in salt form

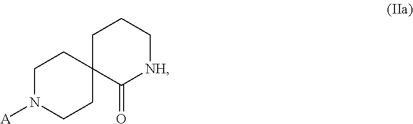

in which A is as defined under formula I, with a compound of the formula IIIa

B-L-Hal (IIIa),

in which B and L are as defined under formula I, and Hal is chloro or bromo, in the presence of a strong base and in the presence of a suitable solvent.

In a further aspect, the invention also provides a process for the production of compounds of the formula Ia in free form or in salt form

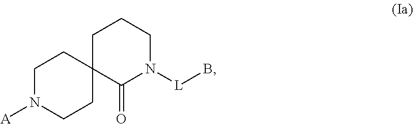

in which A, B and L are as defined under formula I, which comprises reacting a compound of the formula II′a in free form or in salt form

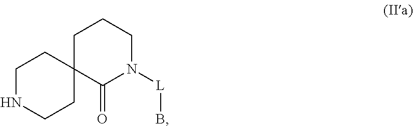

in which L and B are as defined under formula I, with a compound of the formula Va

A-Hal (Va),

in which A is as defined under formula I, and Hal is chloro or bromo, in the presence of a base and in the presence of a suitable solvent.

In a further aspect, the invention also provides a process for the production of compounds of the formula Ib in free form or in salt form

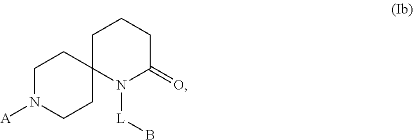

in which A, B and L are as defined under formula I, which comprises reacting a compound of the formula IIb in free form or in salt form

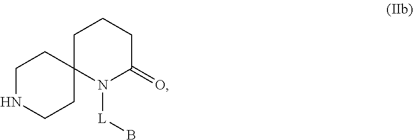

in which B and L are as defined under formula I, with a compound of the formula Va

A-Hal (Va),

in which A is as defined under formula I, and Hal is chloro or bromo, in the presence of a base and in the presence of a suitable solvent.

In another aspect, the invention provides a pharmaceutical composition comprising a compound of the invention and a pharmaceutically acceptable carrier. The pharmaceutical composition can be formulated for particular routes of administration such as oral administration, parenteral administration, and rectal administration, etc. In addition, the pharmaceutical compositions of the invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, solutions, microemulsions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. A further example of formulations for oral use include nanosuspensions comprising an effective amount of a compound of the invention, e.g. about 10% w/w, in water and stabilisers such as hydroxypropylcellulose and sodium dodecyl sulfate typically in an amount of about 1.5% and about 0.05% respectively.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The invention further provides anhydrous pharmaceutical compositions and dosage forms comprising the compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the invention as an active ingredient will decompose. Such agents, which are referred to herein as “stabilizers,” include, but are not limited to, antioxidants such as ascorbic acid, pH buffers, or salt buffers, etc.

As used herein, the term “pharmaceutically acceptable carrier” includes any and all solvents, dispersion media, coatings, surfactants, antioxidants, preservatives (a g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of formula I in free form or in pharmaceutically acceptable salt form, exhibit valuable pharmacological properties, e.g. orexin receptor modulating properties, e.g. as indicated in in-vitro and in-vivo tests as provided in the next sections and are therefore indicated for therapy.

Preferred compounds of formula I show an inhibition of calcium accumulation in recombinant cells expressing at least one of hO×1R or hO×2R at 10 μM of test compound of at least 10%. In one embodiment of the invention, compounds of formula I, which are described in Table 2 as showing an inhibition of calcium accumulation in recombinant cells expressing at least one of hO×1R or hO×2R at 10 μM of test compound of lower than 10%, are excluded.

Further preferred compounds of formula (I) show a Ki value for said calcium accumulation in recombinant cells expressing at least one of hO×1R or hO×2R of at least 1 μM.

Further preferred compounds of formula (I) show a Ki value for said calcium accumulation in recombinant cells expressing at least one of hO×1R or hO×2R of at least 500 nM.

Further preferred compounds of formula (I) show a Ki value for said calcium accumulation in recombinant cells expressing at least one of hO×1R or hO×2R of at least 100 nM.

Further preferred compounds of formula (I) show a Ki value for said calcium accumulation in recombinant cells expressing at least one of hO×1R or hO×2R of at least 50 nM.

Compounds of the invention may be useful in the treatment of an indication selected from:

i) sleep disorders;
ii) eating disorders;
iii) substance-related disorders;
iv) Alzheimers disease;
v) psychiatric, neurological and neurodegenerative disorders, such as depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or haemorrhagic stroke; migraine; and neurodegenerative disorder including nosological entities such as disinhibition-dementia-parkinsonism-amyotrophy complex; pallido-ponto-nigral degeneration epilepsy; seizure disorders;
vi) cardiovascular diseases, diabetes; asthma; Cushing's syndrome/disease; basophil adenoma; prolactinoma; hyperprolactinemia; hypopituitarism; hypophysis tumour/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid hemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and
vii) other diseases related to general orexin system dysfunction.

Compounds of the invention may be especially useful in the treatment of an indication selected from: sleep disorders, eating disorders, substance-related disorders and Alzheimers disease.

“Eating disorders” may be defined as comprising metabolic dysfunction; dysregulated appetite control; compulsive obesities; emeto-bulimia or anorexia nervosa. This pathologically modified food intake may result from disturbed appetite (attraction or aversion for food); altered energy balance (intake vs expenditure); disturbed perception of food quality (high fat or carbohydrates, high palatability); disturbed food availability (unrestricted diet or deprivation) or disrupted water balance.

“Sleep disorders” include insomnias, narcolepsy and other disorders of excessive sleepiness, sleep-related dystonias; restless leg syndrome; sleep apneas; jet-lag syndrome; shift-work syndrome, delayed or advanced sleep phase syndrome. Insomnias are defined as comprising sleep disorders associated with aging; intermittent treatment of chronic insomnia; situational transient insomnia (new environment, noise) or short-term insomnia due to stress; grief; pain or illness.

“Substance-related disorders” include substance abuse, substance dependence and substance withdrawal disorders, e.g. nicotine withdrawal or narcotics withdrawal.

Thus, as a further embodiment, the invention provides the use of a compound of formula (I) in free form or in pharmaceutically acceptable salt form as a medicament.

As a further embodiment, the invention provides the use of a compound of formula (I) in free form or in pharmaceutically acceptable salt form in therapy.

In a further embodiment, the therapy is selected from a disease which is ameliorated by modulation, preferably antagonism, of orexin receptors. In another embodiment, the disease is selected from the afore-mentioned list, suitably sleep disorders, eating disorders, substance-related disorders or Alzheimers disease.

In another embodiment, the invention provides a method of treating a disease which is ameliorated by modulation, preferably antagonism, of orexin receptors comprising administration of a therapeutically acceptable amount of a compound of formula (I) in free form or in pharmaceutically acceptable salt form. In a further embodiment, the disease is selected from the afore-mentioned list, suitably sleep disorders, eating disorders or Alzheimers disease.

In one embodiment, the invention provides a method of inhibiting orexin receptor activity in a subject, wherein the method comprises administering to the subject a therapeutically effective amount of a compound of formula I.

In a further embodiment, the invention provides a method of treating a disorder or a disease in a subject mediated by orexin receptors, wherein the method comprises administering to the subject a therapeutically effective amount of a compound of formula I. Preferably said disorder or said disease is selected from sleep disorders, eating disorders, substance-related disorders, mental health disorders or Alzheimer's disease.

In yet a further embodiment, the invention provides the use of a compound of formula I, for the treatment of a disorder or disease in a subject mediated by orexin receptors.

In yet a further embodiment, the invention provides the use of a compound of formula I, for the treatment of a disorder or disease in a subject characterized by an abnormal activity of orexin receptors. Preferably said disorder or said disease is selected from sleep disorders, eating disorders, substance-related disorders, mental health disorders or Alzheimer's disease.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by orexin receptors, or (ii) associated with orexin receptor activity, or (iii) characterized by abnormal activity of orexin receptors; or (2) reducing or inhibiting the activity of orexin receptors; or (3) reducing or inhibiting the expression of orexin receptors. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of orexin receptors; or at least partially reducing or inhibiting the expression of orexin receptors.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The pharmaceutical composition or combination of the invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the invention can be assessed by in vitro & in vivo methods described herein.

The compound of the invention may be administered either simultaneously with, or before or after, at least one other therapeutic agent. The compound of the invention may be administered separately, by the same or different route of administration, or together in the same pharmaceutical composition.

The following Examples illustrate the invention, but do not limit it.

### ABBREVIATIONS

Boc tert-butoxycarbonyl
d day(s)
DBU 1,8-diazabicyclo[5.4.0]undec-7-en
1,2-DCE 1,2-dichloroethane
DCM dichloromethane
DIPEA N-ethyl-N-isopropylpropan-2-amine (Diisopropyletyhlamine)
DMAP N,N-dimethylpyridin-4-amine
DMF dimethylformamide
DMSO dimethylsulfoxide
EtOAc ethyl acetate
Et₂O diethyl ether
h hour(s)
Hex hexane
HPLC high pressure liquid chromatography
LCMS liquid chromatography mass spectroscopy
min minute(s)
NMP N-methyl-2-pyrrolidone (1-methyl-2-pyrrolidone)
NMR nuclear magnetic resonance spectrometry
quant. quantitative
Rt retention time
rt room temperature
TBAI tetrabutylammonium iodide
TBME tert-butyl methyl ether
THF tetrahydrofuran
TFA trifluoroacetic acid
Ts Tosyl
UPLC ultra performance liquid chromatography

### LCMS Conditions (%=Percent by Volume):

### Method A (Rt_{A}=Retention Time A)

Acquity UPLC/MS Waters, column Waters Acquity HSS T3 1.8 μm, 2.1×50 mm; A: water+0.05% formic acid+0.05% ammonium acetate/B: acetonitrile+0.04% formic acid; 98% A to 98% B in 1.4 min, 98% B 0.75 min; to 98% A in 0.05 min; flow 1.2 ml/min; column temperature 50° C.

Method B (Rt_{B}=retention time B)

Agilent 1100series; LC-MS; column Zorbax SB-C18 1.8 μm, 3.0×30 mm; A: water+0.05% trifluoroacetic acid I B: acetonitrile+0.05% trifluoroacetic acid in; 100% A to 100% B in 3.25 min, 100% B 0.75 min, to 100% A in 0.25 min; flow 0.7 ml/min; column temperature 35° C.

### Method C (Rt_{c}=Retention Time C)

Agilent 1100series; LC-MS; column Zorbax SB-C18 1.8 μm, 3.0×30 mm; A: water+0.05% trifluoroacetic acid/B: acetonitrile+0.05% trifluoroacetic acid in; 90% A to 100% B in 3.25 min, 100% B 0.75 min, to 90% A in 0.25 min; flow 0.7 ml/min; column temperature 35° C.

### Method D (Rt_{D}=Retention Time D)

Agilent 1100series; LC-MS; column Zorbax SB-C18 1.8 μm, 3.0×30 mm; A: water+0.05% trifluoroacetic acid/B: acetonitrile+0.05% trifluoroacetic acid in; 70% A to 100% B in 3.25 min, 100% B 0.75 min, to 70% A in 0.25 min; flow 0.7 ml/min; column temperature 35° C.

### Method E (Rt_{E}=Retention Time E)

Agilent 1100series; LC-MSD; column Mercury MS Synergi 2μ, 20×4.0 mm; A: water+0.1% formic acid/B-acetonitrile; 0-0.5 min 70A-30B; 1.5-2.4 min 5A-95B; 2.5-3.0 min 70A-30B; flow 2.0 ml/min; column temperature 30° C.

### Method F (Rt_{F}=Retention Time F)

API 2000 series; LC-MSD; column Mercury MS Synergi 2μ, 20×4.0 mm; A: water+0.1% formic acid/B-acetonitrile; 0-0.5 min 70A-30B; 1.5-2.4 min 5A-95B; 2.5-3.0 min 70A-30B; flow 2.0 ml/min; column temperature 30° C.

### Method G (Rt_{G}=Retention Time G)

Agilent 1100series; LC-MS; column Ascentis Express FusedCore 2.1×30 mm 2.7 μm C18; A: water+0.05% trifluoroacetic acid I B: acetonitrile+0.04% trifluoroacetic acid in; 90% A to 95% B in 1.7 min, 95% B 0.7 min, to 90% A in 0.05 min; flow 1.4 ml/min; column temperature 50° C.

¹H-NMR Instruments: Varian Mercury (300 MHz), Bruker BioSpin (600 MHz), Bruker (400 MHz), Varian (400 MHz)

### EXAMPLES

### Method A

### Example 1

### 9-(4,6-dimethylpyrimidin-2-0)-2-(2-(3-methyl-1,2,4-oxadiazol-5-yl)benzyl)-2,9-diazaspiro[5.5]undecan-1-one

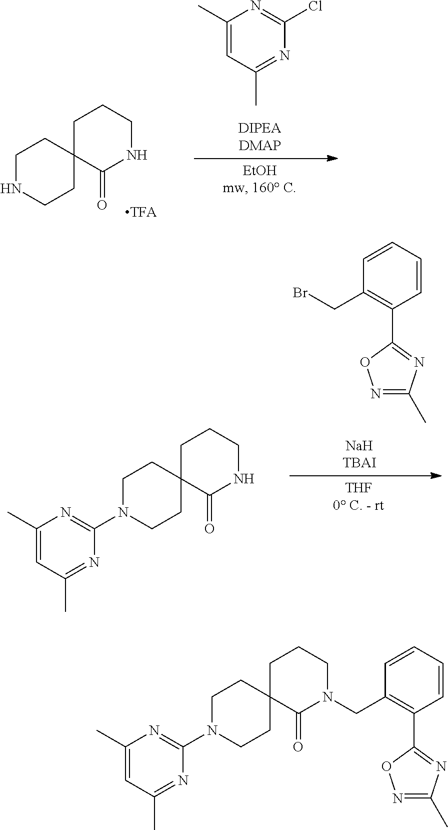

### a) 9-(4,6-dimethylpyrimidin-2-yl)-2,9-diazaspiro[5.5]undecan-1-one

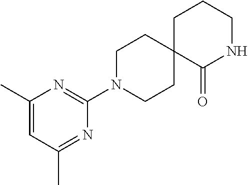 [SMILES: Cc1cc(C)nc(N2CCC3(CCCNC3=O)CC2)n1]

To a solution of 2,9-diazaspiro[5.5]undecan-1-one (TFA salt) (1.4 g, 4.96 mmol) in ethanol (11 mL) was added 2-chloro-4,6-dimethylpyrimidine (0.875 g, 6.0 mmol), DIPEA (4.3 mL, 25 mmol) and DMAP (30 mg, 0.25 mmol) in a microwave tube. The tube was sealed and the suspension was heated at 160′C over 2 h under microwave conditions. The solvent was removed under reduced pressure and the resulting crude product was taken up in ethanol, filtered and the solid washed with ethanol. The filtrate was concentrated, the precipitate from the filtrate was filtered, washed with ethanol and the combined solids were taken up in ethyl acetate. The ethyl acetate layer was washed with water and brine, and dried over anhydrous sodium sulfate. The organic layer was filtered and concentrated under reduced pressure to give 1.02 g (75%) of a white solid. No further purification was required. [¹H NMR (600 MHz, DMSO-d₆) δ 7.34 (br. s., 1H), 6.36 (s, 1H), 4.19-4.38 (m, 2H), 3.23 (t, J=11.10 Hz, 2H), 3.03-3.15 (m, 2H), 2.21 (s, 6H), 1.81-1.92 (m, 2H), 1.75-1.81 (m, 2H), 1.63-1.73 (m, 2H), 1.40 (d, J=13.32 Hz, 2H); LCMS Rt_{B}=2.56 min, [M+H]⁺=275.2].

### b) 9-(4,6-dimethylpyrimidin-2-yl)-2-(2-(3-methyl-1,2,4-oxadiazol-5-yl)benzyl)-2,9-diazaspiro[5.5]undecan-1-one

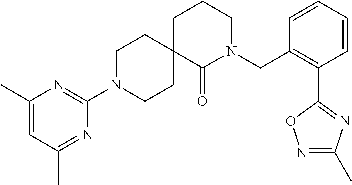

To a suspension of 9-(4,6-dimethylpyrimidin-2-yl)-2,9-diazaspiro[5.5]undecan-1-one (40 mg, 0.146 mmol) and TBAI (2.7 mg, 7.3 μmol) in dry THF (1 mL) was added at 0° C. under argon sodium hydride (95%, 7.4 mg, 0.29 mmol). After stirring for 20 min a solution of 5-(2-(bromomethyl)phenyl)-3-methyl-1,2,4-oxadiazole (46 mg, 0.18 mmol) in dry THF (0.4 mL) was added. The mixture was allowed to warm to room temperature and stirred for 2 h. Water was added and the reaction mixture was extracted with ethyl acetate. The organic layer was washed with water and brine and dried over anhydrous sodium sulfate, filtered and concentrated under reduced pressure. The resulting crude product was purified by flash chromatography (EtOAc/hexane 4:6) to yield 57 mg (86%) of the title compound [¹H NMR (600 MHz, DMSO-d6) δ 8.05 (d, J=7.87 Hz, 1H), 7.67 (t, J=7.57 Hz, 1H), 7.50 (t, J=7.47 Hz, 1H), 7.24 (d, J=7.87 Hz, 1H), 6.36 (s, 1H), 4.89 (s, 2H), 4.36 (d, J=13.72 Hz, 2H), 3.21-3.31 (m, 4H), 2.44 (s, 3H), 2.21 (s, 6H), 1.89-2.00 (m, 4H), 1.79-1.89 (m, 2H), 1.54 (d, J=13.52 Hz, 2H); LCMS Rt_{c}=2.97 min, [M+H]⁺=447.2].

### Method B

### Example 2

### 2-(2-(2H-1,2,3-triazol-2-yl)benzyl)-9-(4,6-dimethylpyrimidin-2-yl)-2,9-diazaspiro[5.5]undecan-1-one

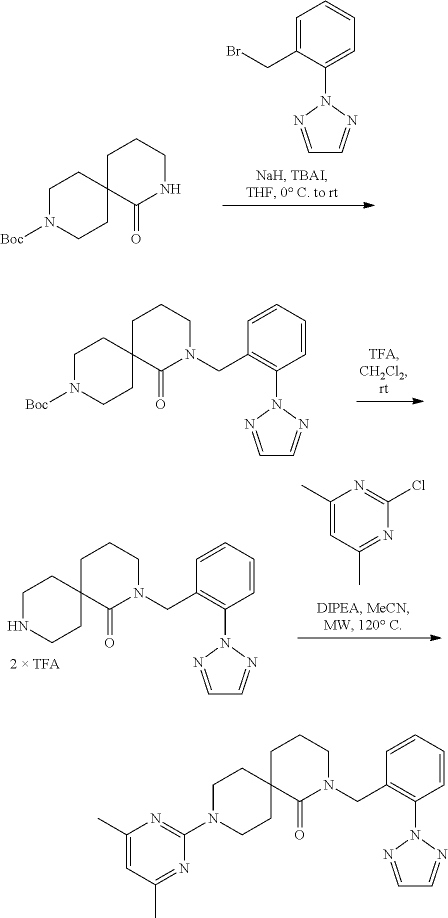

### a) tert-butyl 2-(2-(2H-1,2,3-triazol-2-yl)benzyl)-1-oxo-2,9-diazaspiro[5.5]undecane-9-carboxylate

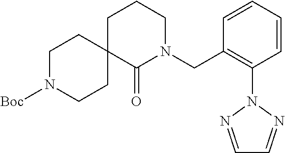 [SMILES: CC(C)(C)OC(=O)N1CCC2(CCCN(Cc3ccccc3-n3nccn3)C2=O)CC1]

NaH (308 mg, 7.69 mmol, 60% in mineral oil) was added to an ice-cold solution of tert-butyl 1-oxo-2,9-diazaspiro[5.5]undecane-9-carboxylate (1.127 g, 4.08 mmol), 2-(2-(bromomethyl)phenyl)-2H-1,2,3-triazole (described separately as building block) (1.0 g, 4.16 mmol) and TBAI (78 mg, 0.208 mmol) in THF (30 mL). The resulting mixture was stirred at 0° C. for 1 h. The reaction mixture was allowed to warm to rt and stirred for 4 h. To the mixture water was added and the solution was extracted twice with ethyl acetate. The organic layer was washed with water and brine, filtered and dried over anhydrous sodium sulfate. The organic layer was concentrated under reduced pressure. The product was purified by flash-column chromatography over silicagel (eluent: gradient 5%-65% ethyl acetate/heptane) to yield the title compound (1.77 g, 99%). [¹H NMR (400 MHz, DMSO-d₆) δ ppm 8.11 (s, 2H), 7.61-7.59 (m, 1H), 7.52-7.46 (m, 2H), 7.23 (d, J=8.78 Hz, 1H), 4.53 (s, 2H), 3.69-3.65 (m, 2H), 3.11-3.07 (m, 2H), 3.03 (br s, 2H), 1.86-1.72 (m, 6H), 1.45-1.40 (m, 2H), 1.38 (s, 9H); LCMS Rt_{A}=1.17, [M+H]⁺=426.4].

### b) 2-(2-(2H-1,2,3-triazol-2-yl)benzyl)-2,9-diazaspiro[5.5]undecan-1-one (TFA salt)

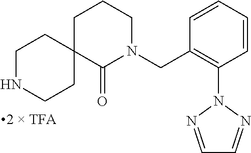 [SMILES: O=C1N(Cc2ccccc2-n2nccn2)CCCC12CCNCC2]

To a solution of tert-butyl 2-(2-(2H-1,2,3-triazol-2-yl)benzyl)-1-oxo-2,9-diazaspiro[5.5]-undecane-9-carboxylate (1.76 g, 4.09 mmol) in dichloromethane (15 mL) was added TFA (3.15 mL, 40.9 mmol). The solution was stirred for 25 min at rt. After completion of the reaction the mixture was evaporated to dryness. The residue was dried under high vacuum to yield the title compound as a solid (2.79 g, quant.). [¹H NMR (400 MHz, DMSO-d₆) δ ppm 8.42 (br s, 2H), 8.11 (s, 2H), 7.64-7.60 (m, 1H), 7.51-7.47 (m, 2H), 7.24 (d, J=4 Hz, 1H), 4.55 (s, 2H), 3.24-3.18 (m, 2H), 3.13-3.11 (m, 2H), 3.09-2.97 (m, 2H), 2.13-2.04 (m, 2H), 1.82-1.70 (m, 4H), 1.65-1.55 (m, 2H); LCMS Rt_{A}=0.60, [M+H]⁺=326.3].

### c) 2-(2-(2H-1,2,3-triazol-2-yl)benzyl)-9-(4,6-dimethylpyrimidin-2-yl)-2,9-diazaspiro[5.5]undecan-1-one

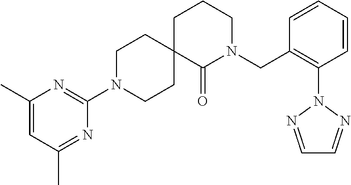 [SMILES: Cc1cc(C)nc(N2CCC3(CCCN(Cc4ccccc4-n4nccn4)C3=O)CC2)n1]

To a stirred solution of 2-(2-(2H-1,2,3-triazol-2-yl)benzyl)-2,9-diazaspiro[5.5]undecan-1-one (TFA salt) (200 mg, 0.354 mmol) and diisopropylethylamine (0.64 mL, 3.61 mmol) in acetonitrile (1 mL) was added 2-chloro-4,6-dimethylpyrimidine [4472-44-0] (115 mg, 0.782 mmol). The mixture was heated at 120° C. in the microwave for 40 min. The reaction mixture was cooled to rt and concentrated under reduced pressure. The crude mixture was purified by flash-column chromatography over silicagel (eluent: gradient 5%-65% ethyl acetate/heptane) to yield the title compound (79 mg, 51%). [¹H NMR (400 MHz, DMSO-d₆) δ ppm 8.11 (s, 2H), 7.62-7.59 (m, 1H), 7.51-7.46 (m, 2H), 7.24 (d, J=8 Hz, 1H), 6.35 (s, 1 H), 4.53 (s, 2H), 4.36-4.28 (m, 2H), 3.26-3.20 (m, 2H), 3.15-3.10 (m, 2H), 2.20 (s, 6 H), 1.90-1.75 (m, 6 H), 1.51-1.40 (m, 2H); LCMS Rt_{A}=1.14, [M+H]⁺=432.4].

### Method C

### Example 3

### 1-((1H-indol-4-yl)methyl)-9-(4,6-dimethylpyrimidin-2-yl)-1,9-diazaspiro[5.5]undecan-2-one

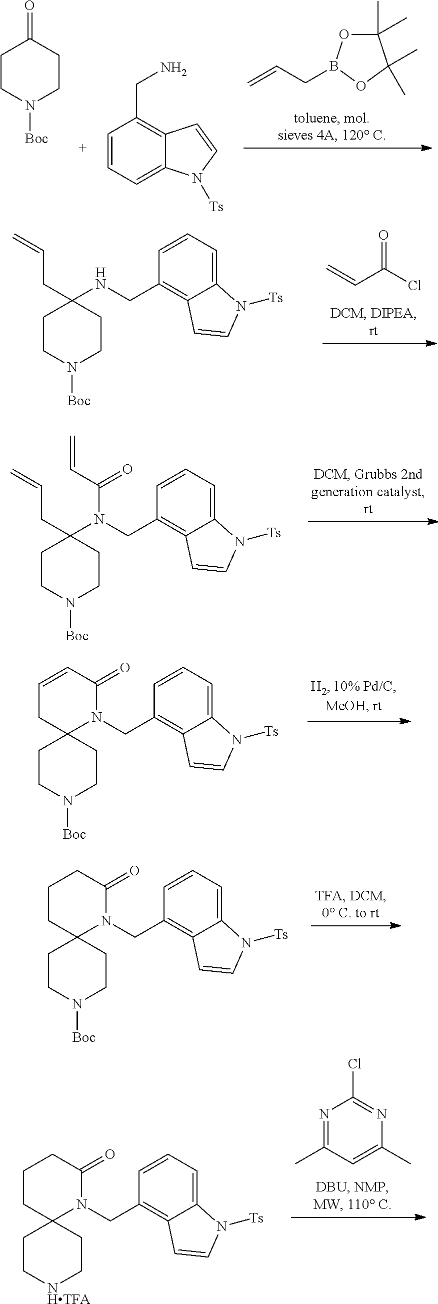

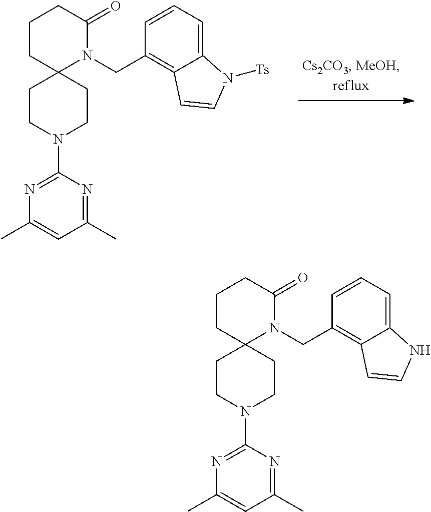 [SMILES: Cc1ccc(S(=O)(=O)n2ccc3c(CN4C(=O)CCCC45CCN(c4nc(C)cc(C)n4)CC5)cccc32)cc1 ; Cc1cc(C)nc(N2CCC3(CCCC(=O)N3Cc3cccc4[nH]ccc34)CC2)n1]

### a) tert-butyl 4-allyl-4-((1-tosyl-1H-indol-4-yl)methylamino)piperidine-1-carboxylate

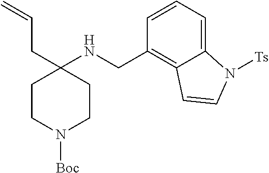 [SMILES: C=CCC1(NCc2cccc3c2ccn3S(=O)(=O)c2ccc(C)cc2)CCN(C(=O)OC(C)(C)C)CC1]

To a stirred mixture of 1-Boc-piperidin-4-one (0.25 g, 1.256 mmol), 4 Å molecular sieves (0.25 g), allyl boronic acid pinacol ester (0.255 g, 1.507 mmol) in toluene (10.0 mL), 1-tosyl-1H-indol-4-yl)methanamine (0.45 g, 1.507 mmol) was added and the reaction mixture was heated to reflux for 16 h. The mixture was filtered through a pad of celite. The filtrate was concentrated under reduced pressure and the residue was purified by column chromatography (eluent: 10% ethyl acetate in hexane) to yield the title compound as a white solid (0.2 g, 70%). [LCMS Rt_{E}=0.341, [M+H]⁺=524.0]

### b) tert-butyl 4-allyl-4-(N-((1-tosyl-1H-indol-4-yl)methyl)acrylamido)piperidine-1-carboxylate

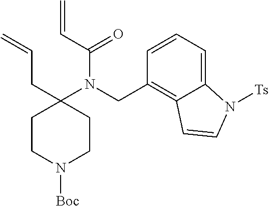 [SMILES: C=CCC1(N(Cc2cccc3c2ccn3S(=O)(=O)c2ccc(C)cc2)C(=O)C=C)CCN(C(=O)OC(C)(C)C)CC1]

Acryloyl chloride (0.360 g, 0.401 mmol) was added at 0° C. to a stirred solution of tert-butyl 4-allyl-4-((1-tosyl-1H-indol-4-yl)methylamino)piperidine-1-carboxylate (0.2 g, 0.382 mmol), diisopropylethylamine (0.32 mL, 1.91 mmol) in dichloromethane (5.0 mL). The reaction mixture was stirred at 0° C. for 30 min and was then allowed to warm to rt and stirred for 4 h. The reaction mixture was concentrated under reduced pressure and the crude product was purified by column chromatography (eluent: 5% ethyl acetate in hexane) to yield the title compound as a white solid (0.16 g, 72%). [LCMS Rt_{E}=0.774, [M+H-Boc]⁺=477.9]

### c) tert-butyl 2-oxo-1-((1-tosyl-1H-indol-4-yl)methyl)-1,9-diazaspiro[5.5]undec-3-ene-9-carboxylate

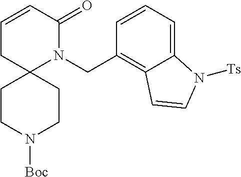 [SMILES: Cc1ccc(S(=O)(=O)n2ccc3c(CN4C(=O)C=CCC45CCN(C(=O)OC(C)(C)C)CC5)cccc32)cc1]

To a solution of tert-butyl 4-allyl-4-(N-((1-tosyl-1H-indol-4-yl)methyl)acrylamido)piperidine-1-carboxylate (0.075 g, 0.13 mmol) in dichloromethane (5.0 mL) was added Grubbs 2^{nd} generation catalyst (0.006 g, 0.006 mmol) under argon and the reaction mixture was stirred at rt overnight. The dark brown solution was concentrated under reduced pressure and the crude product was purified by column chromatography (eluent: 25% ethyl acetate in hexane) to yield the title compound as a solid (0.060 g, 84%). [LCMS Rt_{E}=0.523, [M+H]⁺=549.8]

### d) tert-butyl 2-oxo-1-((1-tosyl-1H-indol-4-yl)methyl)-1,9-diazaspiro[5.5]undecane-9-carboxylate

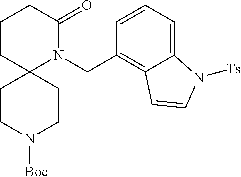 [SMILES: Cc1ccc(S(=O)(=O)n2ccc3c(CN4C(=O)CCCC45CCN(C(=O)OC(C)(C)C)CC5)cccc32)cc1]

To a solution of tert-butyl 2-oxo-1-((1-tosyl-1H-indol-4-yl)methyl)-1,9-diazaspiro[5.5]undec-3-ene-9-carboxylate (0.12 g, 0.218 mmol) in methanol (6.0 mL) was added 10% Pd/C and the reaction mixture was stirred for 6 h under hydrogen (1 atm. pressure) at rt. The reaction mixture was filtered through a pad of celite and washed with methanol. The filtrate was concentrated and the product was isolated as a white solid (0.120 g, 99%). [LCMS Rt_{E}=0.511, [M+H]⁺=551.9]

### e) 1-((1-tosyl-1H-indol-4-yl)methyl)-1,9-diazaspiro[5.5]undecan-2-one (TFA salt)

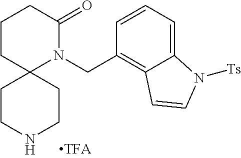 [SMILES: Cc1ccc(S(=O)(=O)n2ccc3c(CN4C(=O)CCCC45CCNCC5)cccc32)cc1]

To a stirred solution of tert-butyl 2-oxo-1-((1-tosyl-1H-indol-4-yl)methyl)-1,9-diazaspiro[5.5]undecane-9-carboxylate (0.12 g, 0.21 mmol) in dichloromethane (5.0 mL), TFA (0.5 mL) was added at 0° C. and the reaction mixture was stirred for 16 h at rt under a nitrogen atmosphere. The reaction mixture was concentrated to yield the title compound as colourless oil (0.11 g, 95%) which was used in the next step.

### f) 9-(4,6-dimethylpyrimidin-2-yl)-1-((1-tosyl-1H-indol-4-yl)methyl)-1,9-diazaspiro[5.5]undecan-2-one

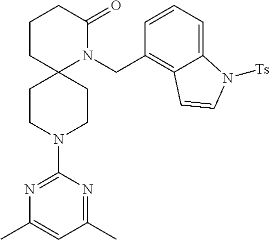 [SMILES: Cc1ccc(S(=O)(=O)n2ccc3c(CN4C(=O)CCCC45CCN(c4nc(C)cc(C)n4)CC5)cccc32)cc1]

To a stirred solution of 1-((1-tosyl-1H-indol-4-yl)methyl)-1,9-diazaspiro[5.5]undecan-2-one (TFA salt) (125 mg, 0.210 mmol) in 1 mL NMP, 2-chloro-4,6-dimethylpyrimidine (37 mg, 0.252 mmol) and DBU (0.112 mL, 0.735 mmol) were added and the reaction mixture was stirred for 25 min at 110° C. in the microwave. The reaction mixture was quenched with saturated NaHCO₃ solution and extracted with TBME (2×25 mL). The organic layer was washed with saturated NH₄Cl solution and brine, then dried over anhydrous Na₂SO₄, filtered and concentrated under reduced pressure. Preparative HPLC (eluent: gradient ethyl acetate/heptane) yielded the title compound as a white foam (67 mg, 57%). [¹H NMR (400 MHz, CHLOROFORM-d) δ ppm 7.82 (d, J=8.28 Hz, 1H), 7.74 (d, J=8.53 Hz, 2H), 7.54 (d, J=3.76 Hz, 1H), 7.22 (d, J=7.78 Hz, 3H), 6.89 (d, J=7.53 Hz, 1H), 6.62 (d, J=4.27 Hz, 1H), 6.26 (s, 1H), 4.80 (br. s., 2H), 4.71 (d, J=18.07 Hz, 2H), 2.93-2.85 (m, 2H), 2.60 (t, J=6.78 Hz, 2H), 2.35 (s, 3H), 2.24 (s, 6 H), 2.15-2.12 (m, 2H), 1.96-1.89 (m, 4H), 1.63 (br. s., 1 H), 1.60 (br. s., 1H), 1.53 (s, 1H), 1.28-1.25 (m, 1H); LCMS Rt_{A}=1.32, [M+H]⁺=558.4].

### q) 1-((1H-indol-4-yl)methyl)-9-(4,6-dimethylpyrimidin-2-yl)-1,9-diazaspiro[5.5]undecan-2-one

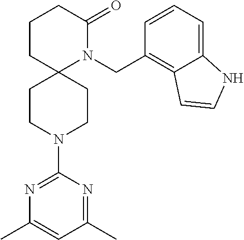 [SMILES: Cc1cc(C)nc(N2CCC3(CCCC(=O)N3Cc3cccc4[nH]ccc34)CC2)n1]

Cs₂CO₃ (137 mg, 0.415 mmol) was added to a stirred solution of 9-(4,6-dimethylpyrimidin-2-yl)-1-((1-tosyl-1H-indol-4-yl)methyl)-1,9-diazaspiro[5.5]undecan-2-one (55 mg, 0.098 mmol) in methanol (2 mL) and stirring was continued for 18 h at 78° C. The reaction mixture was quenched with ice-cold water and extracted with ethyl acetate (2×50 mL), dried over anhydrous Na₂SO₄, filtered and concentrated. The crude product was purified by flash column chromatography (eluent: ethyl acetate) to yield the title compound as a white foam (37 mg, 94%). [¹H NMR (400 MHz, DMSO-d₆) δ ppm 7.26 (t, J=2.76 Hz, 1H), 7.20 (d, J=8.28 Hz, 1H), 6.96 (t, J=7.65 Hz, 1H), 6.62 (d, J=7.03 Hz, 1H), 6.43 (br. s., 1H), 6.32 (s, 1H), 4.72 (br. s., 2H), 4.54 (d, J=13.80 Hz, 2H), 2.89 (t, J=12.42 Hz, 2H), 2.43 (t, J=6.53 Hz, 2H), 2.14 (s, 6 H), 2.09 (br. s., 2H), 1.83-1.75 (m, 4H), 1.55 (d, J=12.80 Hz, 2H); LCMS Rt_{A}=1.01, [M+H]⁺=404.4].

### Method D

### Example 4

### 2-((1H-indol-3-yl)methyl)-9-(4-methoxy-6-methylpyrimidin-2-yl)-2,9-diazaspiro[5.5]undecan-1-one

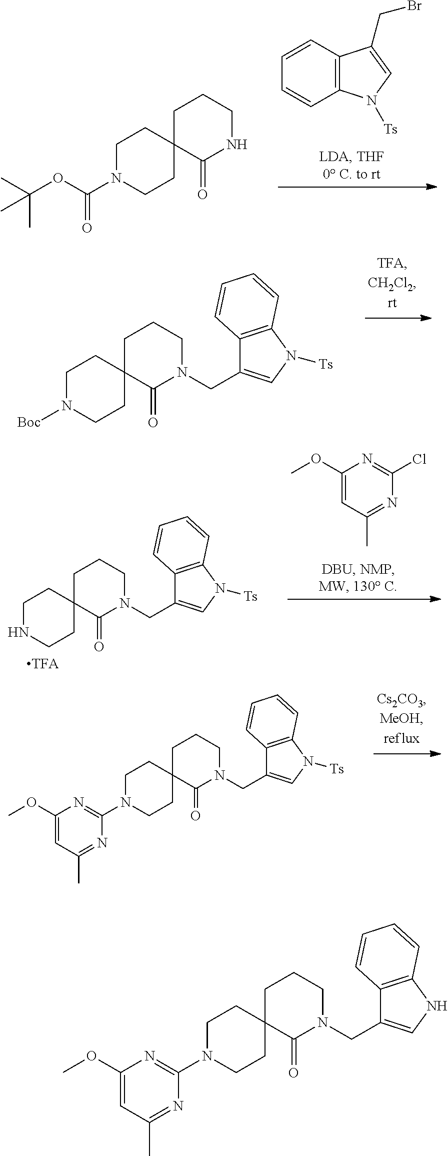

### a) tert-butyl 1-oxo-2-((1-tosyl-1H-indol-3-yl)methyl)-2,9-diazaspiro[5.5]undecane-9-carboxylate

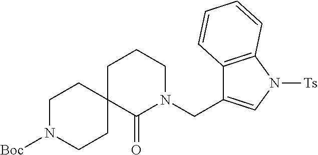 [SMILES: Cc1ccc(S(=O)(=O)n2cc(CN3CCCC4(CCN(C(=O)OC(C)(C)C)CC4)C3=O)c3ccccc32)cc1]

To a solution of diisopropylamine (1.238 mL, 8.60 mmol) in THF (40 mL) n-butyllithium (6.01 mL, 9.61 mmol) was added at 0° C. and the mixture was stirred for 30 min at 0° C. Then a solution of tert-butyl 1-oxo-2,9-diazaspiro[5.5]undecane-9-carboxylate (2.57 g, 9.28 mmol) in THF (10 mL) was added within 3 min and the mixture was stirred for 30 min at 0° C. 3-(bromomethyl)-1-tosyl-1H-indole (3.2 g, 8.43 mmol) in THF (10 mL) was dropped to the reaction mixture within 15 min. The mixture was stirred at 0° C. for 1 h and allowed to warm to room temperature overnight. The reaction mixture was quenched with ice-cold water and extracted with TBME (2×150 mL). The combined organic layers were washed with 5% aqueous citric acid and brine, dried over anhydrous Na₂SO₄, filtered and concentrated (5.3 g, 100%). NMR (400 MHz, DMSO-d₆) δ ppm 7.88 (d, J=8.28 Hz, 1H), 7.81 (d, J=8.28 Hz, 2 H), 7.74 (s, 1H), 7.55 (d, J=7.78 Hz, 1H), 7.36 (d, J=8.03 Hz, 2H), 7.32 (t, J=7.91 Hz, 1H), 7.25-7.21 (m, 1H), 4.58 (s, 2H), 3.75-3.63 (m, 2H), 3.09 (t, J=5.77 Hz, 2H), 2.99 (br. s., 2 H), 2.30 (s, 3H), 1.90-1.80 (m, 2H), 1.72-1.57 (m, 4H), 1.39 (s, 9 H), 1.36-1.28 (m, 2H); LCMS Rt_{A}=1.37, [M+H]⁺=552.3].

### b) 2-((1-tosyl-1H-indol-3-yl)methyl)-2,9-diazaspiro[5.5]undecan-1-one (TFA salt)

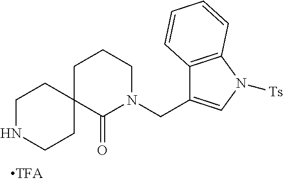 [SMILES: Cc1ccc(S(=O)(=O)n2cc(CN3CCCC4(CCNCC4)C3=O)c3ccccc32)cc1]

To a solution of tert-butyl 1-oxo-2-((1-tosyl-1H-indol-3-yl)methyl)-2,9-diazaspiro[5.5]undecane-9-carboxylate (5.3 g, 8.45 mmol) in dichloromethane (30 mL) was added TFA (4.93 mL, 63.4 mmol). The solution was stirred for 70 min at rt. After completion of the reaction the mixture was evaporated to dryness. The residue was crystallized in THF/heptane 3:1 to yield the title compound as white crystals (5.5 g, quant.). [¹H NMR (400 MHz, DMSO-d₆) δ ppm 8.43 (br. s, 2H), 7.89 (d, J=8.28 Hz, 1H), 7.82 (d, J=8.28 Hz, 2H), 7.78 (s, 1H), 7.56 (d, J=7.78 Hz, 1H), 7.38-7.31 (m, 3H), 7.24-7.20 (m, 1H), 4.59 (s, 2H), 3.29-3.19 (m, 2H), 3.12 (t, J=5.77 Hz, 2H), 3.07-2.95 (m, 2H), 2.30 (s, 3H), 2.10 (ddd, J=14.24, 10.35, 4.02 Hz, 2H), 1.75-1.59 (m, 4H), 1.58-1.48 (m, 2H); LCMS Rt_{A}=0.88, [M+H]⁺=452.3].

### c) 9-(4-methoxy-6-methylpyrimidin-2-yl)-2-((1-tosyl-1H-indol-3-yl)methyl)-2,9-diazaspiro[5.5]undecan-1-one

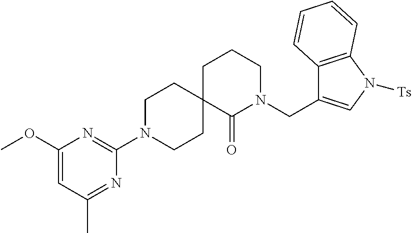 [SMILES: COc1cc(C)nc(N2CCC3(CCCN(Cc4cn(S(=O)(=O)c5ccc(C)cc5)c5ccccc45)C3=O)CC2)n1]

To a stirred solution of 2-((1-tosyl-1H-indol-3-yl)methyl)-2,9-diazaspiro[5.5]undecan-1-one (TFA salt) (130 mg, 0.197 mmol) in 1 mL NMP, 2-chloro-4-methoxy-6-methylpyrimidine (45.2 mg, 0.257 mmol) and DBU (0.11 mL, 0.73 mmol) were added and the reaction mixture was stirred for 20 min at 130° C. in the microwave. The reaction mixture was quenched with saturated NaHCO₃ solution and extracted with TBME (2×25 mL). The organic layer was washed with saturated NH₄Cl solution and brine, then dried over anhydrous Na₂SO₄, filtered and concentrated under reduced pressure. The crude mixture was purified by flash-column chromatography over silicagel (eluent: gradient 10%-100% ethyl acetate/heptane) to yield the title compound (70 mg, 61%). [¹H NMR (400 MHz, DMSO-d₆) δ ppm 7.89 (d, J=8.28 Hz, 1H), 7.81 (d, J=8.53 Hz, 2H), 7.75 (s, 1H), 7.56 (d, J=7.78 Hz, 1H), 7.37-7.30 (m, 3H), 7.25-7.21 (m, 1H), 5.90 (s, 1H), 4.59 (s, 2H), 4.36-4.27 (m, 2H), 3.79 (s, 3H), 3.21 (t, J=13.55 Hz, 2H), 3.12 (t, J=5.90 Hz, 2H), 2.30 (s, 3H), 2.17 (s, 3H), 1.98-1.88 (m, 2H), 1.79-1.73 (m, 2H), 1.70-1.61 (m, 2H), 1.44-1.35 (m, 2H); LCMS Rt_{A}=1.35, [M+H]+=574.4].

### d) 2-((1H-indol-3-yl)methyl)-9-(4-methoxy-6-methylpyrimidin-2-yl)-2,9-diazaspiro[5.5]undecan-1-one

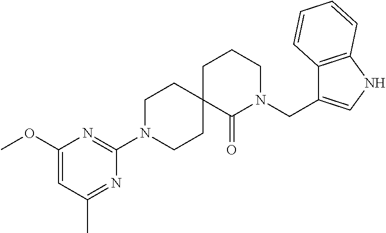 [SMILES: COc1cc(C)nc(N2CCC3(CCCN(Cc4c[nH]c5ccccc45)C3=O)CC2)n1]

Cs₂CO₃ (165 mg, 0.507 mmol) was added to a stirred solution of 9-(4-methoxy-6-methylpyrimidin-2-yl)-2-((1-tosyl-1H-indol-3-yl)methyl)-2,9-diazaspiro[5.5]undecan-1-one (66 mg, 0.113 mmol) in methanol (2 mL) and stirring was continued for 4 h at 80° C. The reaction mixture was quenched with ice-cold water and extracted with dichloromethane (2×50 mL), dried over anhydrous Na₂SO₄, filtered and concentrated to yield the title compound (33 mg, 70%). [¹H NMR (400 MHz, DMSO-d₆) δ ppm 7.52 (d, J=8.03 Hz, 1H), 7.33 (d, J=8.03 Hz, 1 H), 7.27 (d, J=2.26 Hz, 1H), 7.05 (t, J=8.16 Hz, 1H), 6.96-6.92 (m, 1H), 5.91 (s, 1H), 4.61 (s, 2H), 4.39-4.31 (m, 2H), 3.80 (s, 3H), 3.25-3.11 (m, 4H), 2.18 (s, 3H), 2.03-1.92 (m, 2 H), 1.80-1.74 (m, 2H), 1.69-1.61 (m, 2H), 1.45-1.37 (m, 2H); LCMS Rt_{A}=0.98, [M+H]⁺=420.4].

### Example 5

### 2-((1H-indol-3-yl)methyl)-9-(4,6-dimethylpyrimidin-2-yl)-2,9-diazaspiro[5.5]undecan-1-one

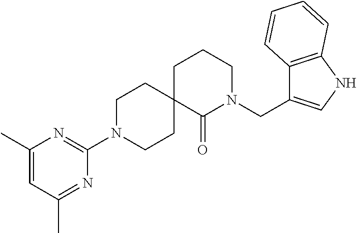 [SMILES: Cc1cc(C)nc(N2CCC3(CCCN(Cc4c[nH]c5ccccc45)C3=O)CC2)n1]

The title compound was synthesized according to method D from 2-((1-tosyl-1H-indol-3-yl)methyl)-2,9-diazaspiro[5.5]undecan-1-one (TEA salt) (see example 4b) and 2-chloro-4,6-dimethylpyrimidine.

[¹H NMR (400 MHz, DMSO-d₆) δ ppm 7.52 (d, J=8.03 Hz, 1H), 7.33 (d, J=8.03 Hz, 1H), 7.27 (d, J=2.01 Hz, 1H), 7.05 (t, J=7.53 Hz, 1H), 6.96-6.92 (m, 1H), 6.36 (s, 1H), 4.61 (s, 2H), 4.42-4.33 (m, 2H), 3.23-3.09 (m, 4H), 2.21 (s, 6 H), 2.01-1.90 (m, 2H), 1.81-1.73 (m, 2H), 1.70-1.60 (m, 2H), 1.45-1.33 (m, 2H); LCMS Rt_{A}=1.11, [M+H]⁺=404.4].

### Example 6

### 9-(4,6-dimethylpyrimidin-2-yl)-2-((1-methyl-1H-indol-3-yl)methyl)-2,9-diazaspiro[5.5]undecan-1-one

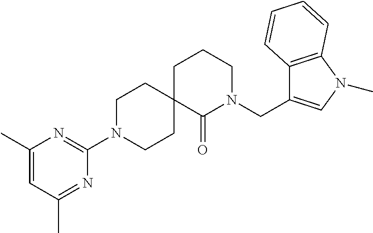

The title compound was synthesized from 2-((1H-indol-3-yl)methyl)-9-(4,6-dimethylpyrimidin-2-yl)-2,9-diazaspiro[5.5]undecan-1-one (example 5) via methylation as follows: NaH (10 mg, 0.26 mmol, 60% in mineral oil) was added to an ice-cold solution of 2-((1H-indol-3-yl)methyl)-9-(4,6-dimethylpyrimidin-2-yl)-2,9-diazaspiro[5.5]undecan-1-one (example 5) (69 mg, 0.17 mmol) in THE (3 mL). The resulting mixture was stirred at 0° C. for 10 min. Then methyl iodide (0.02 mL, 0.26 mmol) was added at 0° C. and the reaction mixture was allowed to warm to rt over a period of 18 h. The mixture was poured into water and the solution was extracted twice with ethyl acetate. The combined organic layers were washed with water and brine, filtered and dried over anhydrous sodium sulfate. The organic layer was concentrated under reduced pressure. The product was purified by preparative HPLC (column AG/PP-C18-15/021, flow 20 mL/min, mobile phase 0.1% TFA in water (A): acetonitrile (B) gradient) to yield the title compound (18 mg, 25%). [¹H NMR (400 MHz, CDCl₃) δ ppm 7.67 (d, 1H), 7.36-7.22 (m, 2H), 7.14 (t, 1H), 7.04 (s, 1H), 6.26 (s, 1H), 4.75 (s, 2H), 4.55-4.43 (m, 2H), 3.80 (s, 3H), 3.38-3.22 (m, 4H), 2.28 (s, 6 H), 2.28-2.20 (m, 2H), 1.88-1.81 (m, 2H), 1.80-1.72 (m, 2H), 1.58-1.39 (m, 2H); LCMS Rt_{F}=1.26, [M+H]⁺=418.0].

### Method E

### Example 7

### 1-((1H-indol-3-yl)methyl)-9-(6-methylpyrazin-2-yl)-1,9-diazaspiro[5.5]undecan-2-one

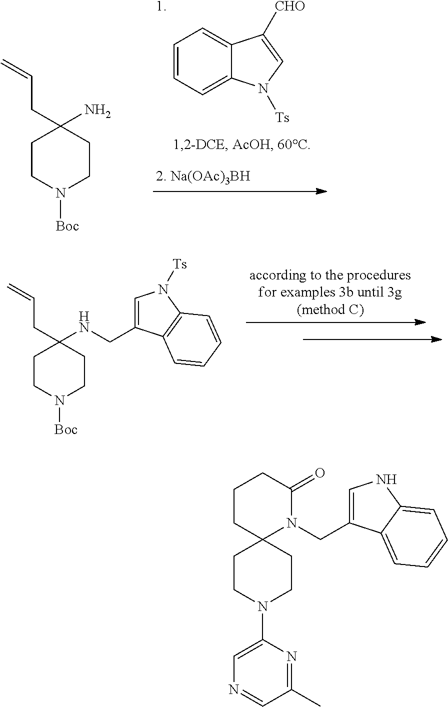 [SMILES: C=CCC1(N)CCN(C(=O)OC(C)(C)C)CC1 ; "1." ; Cc1ccc(S(=O)(=O)n2cc(C=O)c3ccccc32)cc1 ; C=CCC1(NCc2cn(S(=O)(=O)c3ccc(C)cc3)c3ccccc23)CCN(C(=O)OC(C)(C)C)CC1 ; Cc1cncc(N2CCC3(CCCC(=O)N3Cc3c[nH]c4ccccc34)CC2)n1]

### a) tert-butyl 4-allyl-4-((1-tosyl-1H-indol-3-yl)methylamino)piperidine-1-carboxylate

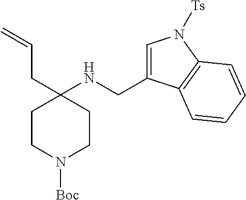 [SMILES: C=CCC1(NCc2cn(S(=O)(=O)c3ccc(C)cc3)c3ccccc23)CCN(C(=O)OC(C)(C)C)CC1]

To a solution of 1-tosyl-1H-indole-3-carbaldehyde (1.90 g, 6.35 mmol) in 1,2-dichloroethane (30 mL) were added tert-butyl 4-allyl-4-aminopiperidine-1-carboxylate (1.52 g, 6.35 mmol) and acetic acid (381 mg, 6.35 mmol). The resulting solution was heated at 60° C. for 3 h. Then NaBH(OAc)₃ was added and the reaction mixture was heated at 60° C. for 38 h. The mixture was cooled to rt, saturated NaHCO₃ solution (30 mL) was added and extracted with ethyl acetate (3×50 mL). The combined organic layers were washed with water and brine, then dried over anhydrous Na₂SO₄, filtered and concentrated under reduced pressure. The crude mixture was purified by flash-column chromatography over silicagel (eluent: 20% ethyl acetate/hexane) to yield the title compound (2.0 g, 60%). [LCMS Rt_{E}=0.34, [M+H]⁺=524.0]

### b) 1-((1H-indol-3-yl)methyl)-9-(6-methylpyrazin-2-yl)-1,9-diazaspiro[5.5]undecan-2-one

The title compound was synthesized from tert-butyl 4-allyl-4-((1-tosyl-1H-indol-3-yl)methylamino)piperidine-1-carboxylate (example 7a) according to the procedures for examples 3b to 3 g (method C).

[¹H NMR (400 MHz, CDCl₃) δ ppm 8.10 (br s, 1H), 7.91 (s, 1H), 7.78 (s, 1H), 7.55 (d, 1H), 7.36 (d, 1H), 7.19 (t, 1H), 7.10 (t, 1H), 4.80 (s, 2H), 4.28-4.15 (m, 2H), 3.05-2.91 (m, 2 H), 2.65-2.55 (t, 2H), 2.38 (s, 3H), 2.31-2.18 (m, 2H), 2.08-2.00 (m, 2H), 1.91-1.80 (m, 2 H), 1.68-1.55 (m, 2H); LCMS Rt_{F}=0.96, [M+H]⁺=390.1].

### Method F

### Example 8

### 2-((1H-pyrrolo[2,3-b]pyridin-3-yl)methyl)-9-(6-methoxypyrazin-2-yl)-2,9-diazaspiro[5.5]undecan-1-one

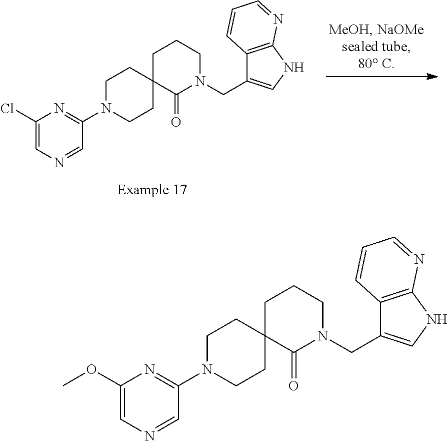 [SMILES: O=C1N(Cc2c[nH]c3ncccc23)CCCC12CCN(c1cncc(Cl)n1)CC2 ; COc1cncc(N2CCC3(CCCN(Cc4c[nH]c5ncccc45)C3=O)CC2)n1]

A stirred solution of 2-((1H-pyrrolo[2,3-b]pyridin-3-yl)methyl)-9-(6-chloropyrazin-2-yl)-2,9-diazaspiro[5.5]undecan-1-one (example 17) (30 mg, 0.073 mmol) and NaOMe (27.6 mg, 0.511 mmol) in MeOH (3 mL) was heated at 90° C. for 18 h. Then the solvent was evaporated under reduced pressure, water was added and the mixture was extracted with ethyl acetate. The combined organic layer was dried over anhydrous sodium sulfate, filtered and concentrated under reduced pressure. The crude mixture was purified by preparative HPLC (column Zorbax eclipse xdb C18 21.2×150 mm 5 μm, flow 20 ml/min, mobile phase 0.1% TFA in water (A): acetonitrile (B) gradient) to yield the title compound (19 mg, 65%). [¹H NMR (CDCl₃, 400 MHz) δ ppm 9.37 (br s, 1H), 8.32 (s, 1H), 8.12 (d, 1H), 7.67 (s, 1H), 7.50 (s, 1H), 7.29 (s, 1H), 7.08-7.15 (m, 1H), 4.72 (s, 2H), 4.15-4.00 (m, 2H), 3.89 (s, 3H), 3.40-3.30 (m, 2H), 3.29-3.22 (m, 2H), 2.35-2.23 (m, 2H), 2.00-1.75 (m, 4H), 1.62-1.50 (m, 2H); LCMS Rt_{E}=0.35, [M+H]⁺=407.2].

### Method G

### Example 9

### 2-((1H-indol-3-yl)methyl)-9-(6-methylpyridin-2-yl)-2,9-diazaspiro[5.5]undecan-1-one

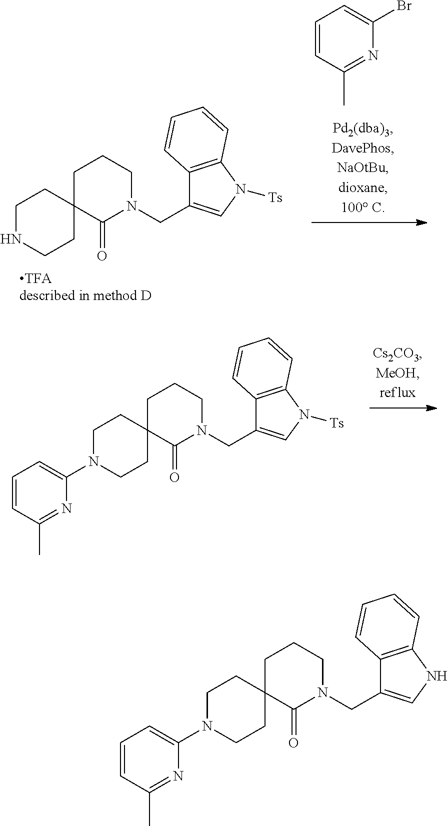

### a) 9-(6-methylpyridin-2-yl)-2-((1-tosyl-1H-indol-3-yl)methyl)-2,9-diazaspiro[5.5]undecan-1-one

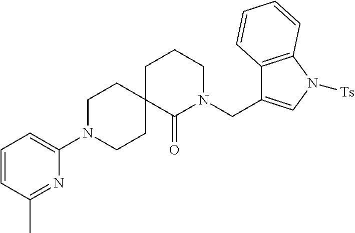 [SMILES: Cc1ccc(S(=O)(=O)n2cc(CN3CCCC4(CCN(c5cccc(C)n5)CC4)C3=O)c3ccccc32)cc1]

The mixture of 2-((1-tosyl-1H-indol-3-yl)methyl)-2,9-diazaspiro[5.5]undecan-1-one (50 mg, 0.077 mmol, contains 1.7 moleq TFA), 2-chloro-6-methylpyridine (13 μl, 0.116 mmol), Pd₂ dba₃ (3.6 mg, 3.9 μmol), sodium t-butanolate (22.3 mg, 0.23 mmol), 2-(2-dicyclohexylphosphanylphenyl)-N,N-dimethylaniline (DavePhos, 3.1 mg, 7.8 μmol) and dry dioxane (1 ml) was placed in a microwave tube and flushed with argon. The tube was sealed and the suspension was heated at 100° C. for 1 h under microwave conditions. The reaction mixture was diluted with ethyl acetate and washed with water and brine and dried over anhydrous sodium sulfate, filtered and concentrated under reduced pressure. The resulting brown oil was purified by flash chromatography (EtOAc/hexane 1:1) to yield 34 mg (80%) of the title compound [LCMS Rt_{D}=2.85 min, [M+H]⁺=543.2].

### b) 2-((1H-indol-3-yl)methyl)-9-(6-methylpyridin-2-yl)-2,9-diazaspiro[5.5]undecan-1-one

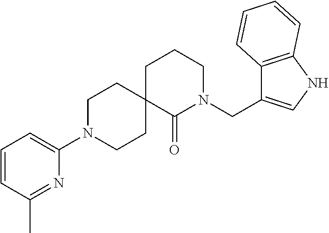 [SMILES: Cc1cccc(N2CCC3(CCCN(Cc4c[nH]c5ccccc45)C3=O)CC2)n1]

The mixture of 9-(6-methylpyridin-2-yl)-2-((1-tosyl-1H-indol-3-yl)methyl)-2,9-diazaspiro[5.5]undecan-1-one (54 mg, 0.1 mmol) and Cs₂CO₃ (162 mg, 0.50 mmol) in methanol (1 ml) was heated under reflux for 18 h. The reaction mixture was diluted with ethyl acetate and washed with water and brine and dried over anhydrous sodium sulfate, filtered and concentrated under reduced pressure to afford the title compound (31 mg, 79%) as a pale beige foam. ¹H NMR (600 MHz, DMSO-d₆) δ ppm 10.95 (br. s., 1H), 7.53 (d, J=7.9 Hz, 1H), 7.39 (t, J=7.8 Hz, 1H), 7.34 (d, J=8.1 Hz, 1H), 7.28 (s, 1H), 7.06 (t, J=7.4 Hz, 1H), 6.95 (t, J=7.5 Hz, 1H), 6.59 (d, J=8.3 Hz, 1H), 6.45 (d, J=7.1 Hz, 1H), 4.62 (s, 2H), 4.04 (d, J=13.3 Hz, 2H), 3.15 (t, J=5.9 Hz, 2H), 3.06 (t, J=11.3 Hz, 2H), 2.29 (s, 3H), 2.04 (td, J=12.4, 4.0 Hz, 2H), 1.72-1.86 (m, 2H), 1.66 (d, J=5.2 Hz, 2H), 1.42 (d, J=13.1 Hz, 2H); LCMS Rt_{c}=2.83 min, [M+H]⁺=389.2].

### Method H

### Example 10

### 2-(2-((1H-indol-3-yl)methyl)-1-oxo-2,9-diazaspiro[5.5]undecan-9-yl)isonicotinonitrile

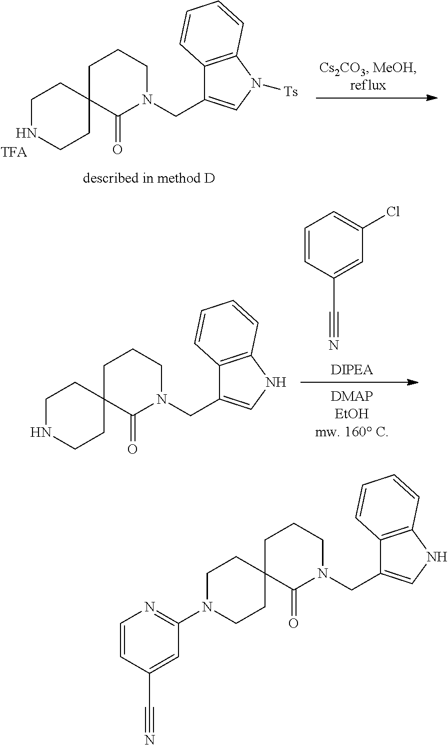 [SMILES: Cc1ccc(S(=O)(=O)n2cc(CN3CCCC4(CCNCC4)C3=O)c3ccccc32)cc1 ; O=C1N(Cc2c[nH]c3ccccc23)CCCC12CCNCC2 ; N#Cc1cccc(Cl)c1 ; N#Cc1ccnc(N2CCC3(CCCN(Cc4c[nH]c5ccccc45)C3=O)CC2)c1]

### a) 2-((1H-indol-3-yl)methyl)-2,9-diazaspiro[5.5]undecan-1-one

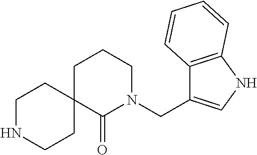 [SMILES: O=C1N(Cc2c[nH]c3ccccc23)CCCC12CCNCC2]

The suspension of 2-((1-tosyl-1H-indol-3-yl)methyl)-2,9-diazaspiro[5.5]undecan-1-one (TFA salt) (15 g, 23.2 mmol) and Cs₂CO₃ (45.4 g, 139 mmol) in methanol (170 ml) was refluxed for 2.5 h. The solution was diluted with water and extracted with CH₂Cl₂. The aqueous phase was adjusted to pH 11 by the addition of saturated aqueous K₂CO₃ solution and extraction with CH₂Cl₂ was repeated. The combined organic layers were dried over sodium sulfate, filtered and evaporated to give 6.96 g (99%) of a beige foam. [¹H NMR (400 MHz, DMSO-d₆) δ ppm 10.91 (br. s., 1H), 7.42-7.61 (m, 1H), 7.32 (d, J=8.2 Hz, 1H), 7.24 (d, J=2.0 Hz, 1 H), 7.04 (t, J=7.6 Hz, 1H), 6.88-6.99 (m, 1H), 4.60 (s, 2H), 3.10 (t, J=6.1 Hz, 2H), 2.51-2.88 (m, 4H), 1.88-2.10 (m, 3H), 1.60 (d, J=5.5 Hz, 4H), 1.25-1.40 (m, 2H); LCMS Rt_{c}=2.44 min, [M+H]⁺=298.2].

### b) 2-(2-((1H-indol-3-yl)methyl)-1-oxo-2,9-diazaspiro[5.5]undecan-9-yl)isonicotinonitrile

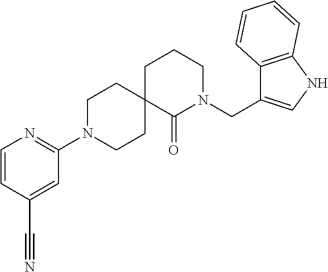 [SMILES: N#Cc1ccnc(N2CCC3(CCCN(Cc4c[nH]c5ccccc45)C3=O)CC2)c1]

To the solution of 2-((1H-indol-3-yl)methyl)-2,9-diazaspiro[5.5]undecan-1-one (50 mg, 0.17 mmol) in ethanol (0.8 ml) was added 2-chloroisonicotinonitrile (36 mg, 0.25 mmol), DIPEA (0.15 ml, 0.84 mmol) and DMAP (1 mg, 0.0084 mmol) in a microwave tube. The tube was sealed and the suspension was heated at 160° C. over 1.5 h under microwave conditions. The solvent was removed under reduced pressure. The resulting crude product was purified by flash chromatography (EtOAc/hexane 9:1) to yield 34 mg (46%) of the title compound CH NMR (600 MHz, DMSO-d₆) δ ppm 10.95 (br. s., 1H), 8.28 (d, J=5.0 Hz, 1H), 7.52 (d, J=7.9 Hz, 1H), 7.34 (d, J=8.1 Hz, 1H), 7.31 (s, 1H), 7.28 (s, 1H), 7.06 (t, J=7.6 Hz, 1H), 6.95 (t, J=7.5 Hz, 1H), 6.90 (d, J=4.8 Hz, 1H), 4.62 (s, 2H), 4.03-4.16 (m, 2H), 3.22 (t, J=11.3 Hz, 2H), 3.16 (t, J=5.9 Hz, 2H), 1.97-2.07 (m, 2H), 1.72-1.83 (m, 2H), 1.60-1.72 (m, 2 H), 1.44 (d, J=13.9 Hz, 2H); LCMS Rt_{D}=2.31 min, [M+H]⁺=400.2].

### Example 11

### 2-((1H-indol-3-yl)methyl)-9-(6-methylpyrazin-2-yl)-2,9-diazaspiro[5.6]undecan-1-one

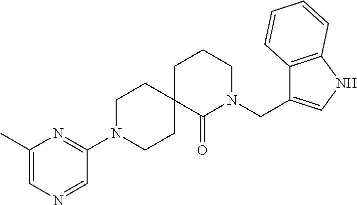 [SMILES: Cc1cncc(N2CCC3(CCCN(Cc4c[nH]c5ccccc45)C3=O)CC2)n1]

The title compound was synthesized according to method H from 2-((1H-indol-3-yl)methyl)-2,9-diazaspiro[5.5]undecan-1-one (see example 10a) and 2-chloro-6-methylpyrazine. [¹H NMR (600 MHz, DMSO-d₆) δ ppm 10.95 (br. s., 1H), 8.10 (s, 1H), 7.69 (s, 1H), 7.53 (d, J=7.9 Hz, 1H), 7.34 (d, J=8.1 Hz, 1H), 7.28 (s, 1H), 7.06 (t, J=7.6 Hz, 1H), 6.95 (t, J=7.4 Hz, 1H), 4.62 (s, 2H), 4.00-4.14 (m, 2H), 3.07-3.24 (m, 4H), 2.30 (s, 3H), 1.99-2.10 (m, 2H), 1.72-1.82 (m, 2H), 1.66 (br. s., 2H), 1.46 (d, J=13.5 Hz, 2H); LCMS Rt_{D}=2.03 min, [M+H]⁺=390.2].

### Example 12

### 2-((1H-indol-3-yl)-methyl)-9-(4-methylpyrimidin-2-yl)-2,9-diazaspiro[5.5]undecan-1-one

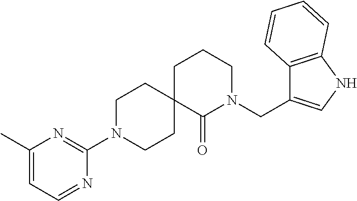 [SMILES: Cc1ccnc(N2CCC3(CCCN(Cc4c[nH]c5ccccc45)C3=O)CC2)n1]

The title compound was synthesized according to method H from 2-((1H-indol-3-yl)methyl)-2,9-diazaspiro[5.5]undecan-1-one (see example 10a) and 2-chloro-4-methylpyrimidine. [¹H NMR (400 MHz, DMSO-d₆) δ ppm 8.19 (d, J=4.77 Hz, 1H), 7.52 (d, J=7.78 Hz, 1H), 7.33 (d, J=8.03 Hz, 1H), 7.27 (d, J=2.26 Hz, 1H), 7.05 (t, J=7.53 Hz, 1H), 6.96-6.92 (m, 1 H), 6.47 (d, J=4.77 Hz, 1H), 4.61 (s, 2H), 4.40-4.31 (m, 2H), 3.26-3.11 (m, 4H), 2.26 (s, 3 H), 2.02-1.91 (m, 2H), 1.82-1.74 (m, 2H), 1.70-1.61 (m, 2H), 1.45-1.37 (m, 2H); LCMS Rt_{A}=1.04, [M+H]⁺=390.2].

### Example 13

### 2-((1H-indol-3-yl)methyl)-9-(4-methoxypyrimidin-2-yl)-2,9-diazaspiro[5.5]undecan-1-one

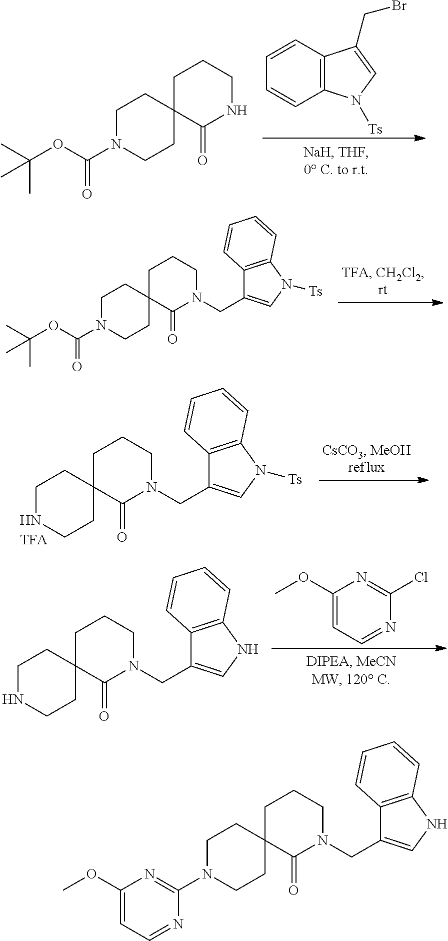

### a) tert-butyl 1-oxo-2-((1-tosyl-1H-indol-3-yl)methyl)-2,9-diazaspiro[5.5]undecane-9-carboxylate

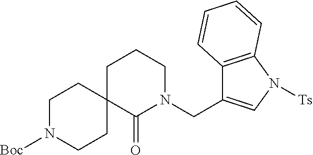 [SMILES: Cc1ccc(S(=O)(=O)n2cc(CN3CCCC4(CCN(C(=O)OC(C)(C)C)CC4)C3=O)c3ccccc32)cc1]

To a solution of 3-(bromomethyl)-1-tosyl-1H-indole (20.1 g, 51.3 mmol), tert-butyl 1-oxo-2,9-diazaspiro[5.5]undecane-9-carboxylate (14.7 g, 54.4 mmol) and tert-butyl ammonium iodide (0.38 g, 1.03 mmol) in THF (200 mL) was added sodium hydride (2.38 g, 57.5 mmol, 58% in dispersion oil) at 0° C. The mixture was stirred for 20 min at 0° C. and allowed to warm to room temperature overnight. The reaction mixture was quenched with ice-cold water and extracted with TBME (3×300 mL). The combined organic layers were washed with brine, dried over anhydrous Na₂SO₄, filtered and concentrated. The residue was crystallized in ethyl acetate/heptane 1:1 (300 mL) to yield the title compound as white crystals (18.1 g, 64%).

[¹H NMR (400 MHz, chloroform-d) δ ppm 7.96 (d, J=8.3 Hz, 1H), 7.75 (d, J=8.3 Hz, 2H), 7.58 (d, J=7.8 Hz, 1H), 7.48 (s, 1H), 7.36-7.29 (m, 1H), 7.25-7.16 (m, 3H), 4.67 (s, 2 H), 3.96-3.76 (m, 2H), 3.22-3.02 (m, 4H), 2.35 (s, 3H), 2.19-2.04 (m, 2H), 1.83-1.67 (m, 4H), 1.47 (s, 9 H), 1.34-1.23 (m, 2H); LCMS Rt_{A}=1.37, [M+H]⁺=552.3].

### b) 2-((1-tosyl-1H-indol-3-yl)methyl)-2,9-diazaspiro[5.5]undecan-1-one (TFA salt)

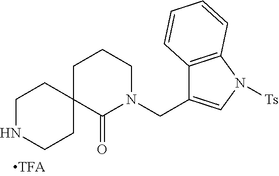 [SMILES: Cc1ccc(S(=O)(=O)n2cc(CN3CCCC4(CCNCC4)C3=O)c3ccccc32)cc1]

To a solution of tert-butyl 1-oxo-2-((1-tosyl-1H-indol-3-yl)methyl)-2,9-diazaspiro[5.5]undecane-9-carboxylate (5.3 g, 8.45 mmol) in dichloromethane (30 mL) was added TFA (4.93 mL, 63.4 mmol). The solution was stirred for 70 min at rt. After completion of the reaction the mixture was evaporated to dryness. The residue was crystallized in THF/heptane 3:1 to yield the title compound as white crystals (5.5 g, 99%). [¹H NMR (400 MHz, DMSO-d₆) δ ppm 8.39 (br. s., 2H), 7.89 (d, J=8.3 Hz, 1H), 7.82 (d, J=8.3 Hz, 2H), 7.78 (s, 1H), 7.56 (d, J=7.8 Hz, 1H), 7.40-7.29 (m, 3H), 7.25-7.17 (m, 1H), 4.59 (s, 2 H), 3.30-3.19 (m, 2H), 3.12 (t, J=5.8 Hz, 2H), 3.07-2.93 (m, 2H), 2.30 (s, 3H), 2.10 (ddd, J=4.0, 10.4, 14.2 Hz, 2H), 1.75-1.59 (m, 4H), 1.58-1.47 (m, 2H); LCMS Rt_{A}=0.88, [M+H]⁺=452.3].

### c) 2-((1H-indol-3-yl)methyl)-2,9-diazaspiro[5.5]undecan-1-one

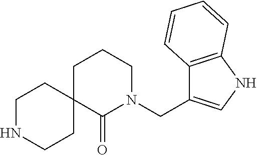 [SMILES: O=C1N(Cc2c[nH]c3ccccc23)CCCC12CCNCC2]

The suspension of 2-((1-tosyl-1H-indol-3-yl)methyl)-2,9-diazaspiro[5.5]undecan-1-one (TFA salt) (15 g, 23.2 mmol) and Cs₂CO₃ (45.4 g, 139 mmol) in methanol (170 ml) was refluxed for 2.5 h. The solution was diluted with water and extracted with CH₂Cl₂. The aqueous phase was adjusted to pH 11 by the addition of saturated aqueous K₂CO₃ solution and extraction with CH₂Cl₂ was repeated. The combined organic layers were dried over sodium sulfate, filtered and evaporated to give 6.96 g (99%) of a beige foam. [¹H NMR (400 MHz, DMSO-d₆) δ ppm 10.91 (br. s., 1H), 7.51 (d, J=7.8 Hz, 1H), 7.32 (d, J=8.2 Hz, 1H), 7.24 (d, J=2.0 Hz, 1H), 7.04 (t, J=7.6 Hz, 1H), 6.97-6.87 (m, 1H), 4.60 (s, 2H), 3.10 (t, J=6.1 Hz, 2 H), 2.89-2.72 (m, 2H), 2.64 (dt, J=2.7, 12.1 Hz, 2H), 2.10-1.89 (m, 3H), 1.73-1.53 (m, 4H), 1.39-1.23 (m, 2H); LCMS Rt_{c}=2.44 min, [M+H]⁺=298.2].

### d) 2-((1H-indol-3-yl)methyl)-9-(4-methoxypyrimidin-2-yl)-2,9-diazaspiro[5.5]undecan-1-one

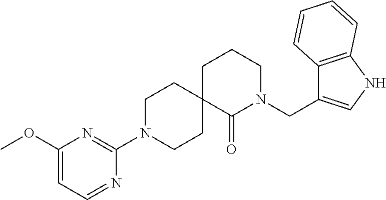 [SMILES: COc1ccnc(N2CCC3(CCCN(Cc4c[nH]c5ccccc45)C3=O)CC2)n1]

To the solution of 2-((1H-indol-3-yl)methyl)-2,9-diazaspiro[5.5]undecan-1-one (3.4 g, 11.32 mmol) in acetonitrile (10 ml) was added 2-chloro-4-methoxypyrimidine (2.17 g, 14.71 mmol), DIPEA (6.99 ml, 39.6 mmol) and DBU (0.052 ml, 0.34 mmol) in a microwave tube. The tube was sealed and the reaction mixture was heated at 120′C over 2 h under microwave conditions. The solvent was removed under reduced pressure. The resulting crude product was purified by flash chromatography (Biotage Isolera Four, heptane/EtOAc 55/45 to heptane/EtOAc 13/87 in 14 min). The product was crystallized from TBME to give the title compound as white crystals (3.33 g, 72%). [¹H NMR (400 MHz, DMSO-d₆) δ ppm 10.93 (br. s., 1H), 8.07 (d, J=5.8 Hz, 1H), 7.52 (d, J=8.0 Hz, 1H), 7.33 (d, J=8.0 Hz, 1H), 7.26 (d, J=2.3 Hz, 1H), 7.05 (t, J=7.5 Hz, 1H), 6.98-6.88 (m, 1H), 6.02 (d, J=5.5 Hz, 1H), 4.61 (s, 2H), 4.33 (td, J=4.1, 13.4 Hz, 2H), 3.82 (s, 3H), 3.29-3.18 (m, 2H), 3.14 (t, J=5.9 Hz, 2H), 2.06-1.90 (m, 2H), 1.82-1.72 (m, 2H), 1.71-1.59 (m, 2H), 1.48-1.35 (m, 2H); LCMS Rt_{A}=0.96, [M+H]⁺=406.3; MP=190-191° C.].

On one occasion, the product was obtained as a TBME hemisolvate.

### Method I

### Example 14

### 6-(1-((1H-indol-4-yl)methyl)-2-oxo-1,9-diazaspiro[5.5]undecan-9-yl)nicotinonitrile

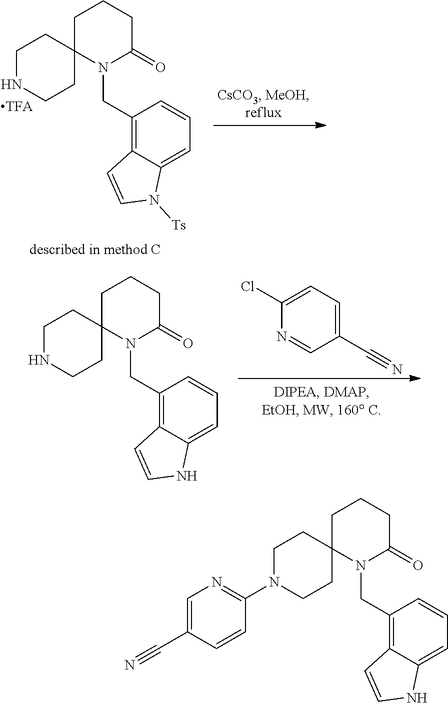 [SMILES: Cc1ccc(S(=O)(=O)n2ccc3c(CN4C(=O)CCCC45CCNCC5)cccc32)cc1 ; O=C1CCCC2(CCNCC2)N1Cc1cccc2[nH]ccc12 ; N#Cc1ccc(Cl)nc1 ; N#Cc1ccc(N2CCC3(CCCC(=O)N3Cc3cccc4[nH]ccc34)CC2)nc1]

### a) 1-((1H-indol-4-yl)methyl)-1,9-diazaspiro[5.5]undecan-2-one

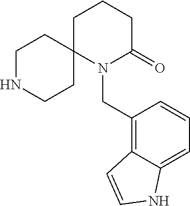 [SMILES: O=C1CCCC2(CCNCC2)N1Cc1cccc2[nH]ccc12]

Cs₂CO₃ (1.1 g, 3.40 mmol) was added to a stirred solution of 1-((1-tosyl-1H-indol-4-yl)methyl)-1,9-diazaspiro[5.5]undecan-2-one (TFA salt) (example 3e) (350 mg, 0.619 mmol) in methanol (7 mL) and stirring was continued for 20 h at 80° C. The reaction mixture was quenched with ice-cold water and the precipitate was filtered off. The filtrate was treated with 1M aqueous NaOH solution and extracted with dichloromethane (3×50 mL), dried over anhydrous Na₂SO₄, filtered and concentrated to yield the title compound (89 mg, 44%). [LCMS Rt_{A}=0.41, [M+H]⁺=298.3].

### b) 6-(1-((1H-indol-4-yl)methyl)-2-oxo-1,9-diazaspiro[5.5]undecan-9-yl)nicotinonitrile

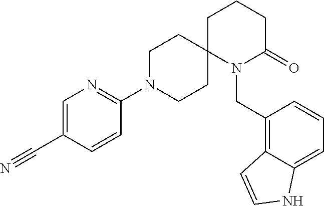 [SMILES: N#Cc1ccc(N2CCC3(CCCC(=O)N3Cc3cccc4[nH]ccc34)CC2)nc1]

To a stirred solution of 1-((1H-indol-4-yl)methyl)-1,9-diazaspiro[5.5]undecan-2-one (40 mg, 0.135 mmol), diisopropylethylamine (0.12 mL, 0.673 mmol) and DMAP (0.82 mg, 6.73 mmol) in ethanol (0.7 mL) was added 6-chloronicotinonitrile (28 mg, 0.202 mmol). The mixture was heated at 160° C. in the microwave for 2 h. The reaction mixture was cooled to it and concentrated under reduced pressure. The crude mixture was purified by flash-column chromatography over silicagel (eluent: dichloromethane/methanol 98:2) to yield the title compound (26 mg, 42%). [¹H NMR (400 MHz, DMSO-d₆) δ ppm 8.39 (d, J=1.76 Hz, 1H), 7.75 (dd, J=9.16, 2.38 Hz, 1H), 7.26 (t, J=2.76 Hz, 1H), 7.19 (d, J=8.03 Hz, 1H), 6.96 (t, J=7.65 Hz, 1H), 6.85 (d, J=9.03 Hz, 1H), 6.62 (d, J=7.03 Hz, 1H), 6.41 (br. s., 1H), 4.72 (br. s., 2H), 4.26 (d, J=12.55 Hz, 2H), 3.04 (t, J=12.17 Hz, 2H), 2.43 (t, J=6.65 Hz, 2H), 2.15-2.06 (m, 2H), 1.94-1.77 (m, 4H), 1.61 (d, J=13.55 Hz, 2H); LCMS R₁₋₄=0.94, [M+H]⁺=400.3].

### Method K

### Example 15

### 9-(4,6-dimethylpyrimidin-2-yl)-2-(2-(furan-2-yl)benzyl)-2,9-diazaspiro[5.5]undecan-1-one

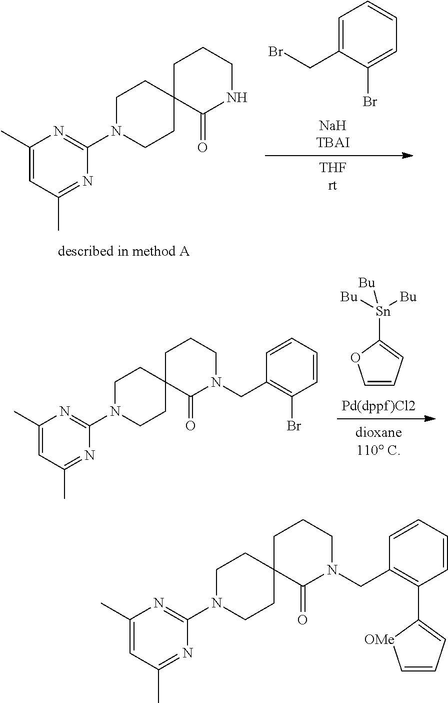

### a) 2-(2-bromobenzyl)-9-(4,6-dimethylpyrimidin-2-yl)-2,9-diazaspiro[5.5]undecan-1-one

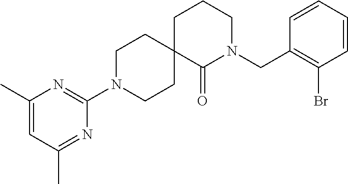 [SMILES: Cc1cc(C)nc(N2CCC3(CCCN(Cc4ccccc4Br)C3=O)CC2)n1]

To a suspension of 9-(4,6-dimethylpyrimidin-2-yl)-2,9-diazaspiro[5.5]undecan-1-one (200 mg, 0.73 mmol) and TBAI (27 mg, 0.073 μmol) in dry THF (6 mL) was added at 0° C. under argon sodium hydride (95%, 37 mg, 1.5 mmol). After stirring for 20 min a solution of 1-bromo-2-(bromomethyl)benzene (98%, 204 mg, 0.80 mmol) in dry THF (1.0 mL) was added. The mixture was allowed to warm to room temperature and stirred for 1.5 h. Water was added and the reaction mixture was extracted with diethyl ether. The organic layer was washed with water and brine and dried over anhydrous sodium sulfate, filtered and concentrated under reduced pressure to afford the title compound (306 mg, 95%) [LCMS Rt_{D}=2.31 min, [M+H]⁺=443.0/445.0].

### b) 9-(4,6-dimethylpyrimidin-2-yl)-2-(2-(furan-2-yl)benzyl)-2,9-diazaspiro[5.5]undecan-1-one

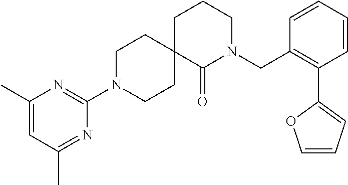 [SMILES: Cc1cc(C)nc(N2CCC3(CCCN(Cc4ccccc4-c4ccco4)C3=O)CC2)n1]

To a mixture of 2-(2-bromobenzyl)-9-(4,6-dimethylpyrimidin-2-yl)-2,9-diazaspiro[5.5]undecan-1-one (50 mg, 0.11 mmol) and PdCl₂(dppf) (CH₂Cl₂ adduct) (9.2 mg, 0.011 mmol) in dioxane (1 mL) was added under Argon 2-(tributylstannyl)furan (97%, 0.073 mL, 0.27 mmol). The reaction vessel was sealed and heated at 100° C. for 4 h. The reaction mixture was concentrated under reduced pressure.

Methanol was added and the mixture filtered through PL-thiol MP SPE cartridge (previously washed with MeOH) and evaporated to give 100 mg of a pale yellow oil which was purified by preparative HPLC (column Waters Sunfire C18, 5 um, 4.6×50 mm, flow 5 mL/min, solvent A: Water+0.1% TFA; Solvent B: Acetonitrile+0.1% TFA/gradient 5-100% B in 2.5 min) to yield the title compound (30 mg, 62%). [¹H NMR (600 MHz, DMSO-d₆) δ ppm 7.81 (s, 1H), 7.59-7.72 (m, 1H), 7.35 (s, 2H), 7.02-7.18 (m, 1H), 6.73 (d, J=3.2 Hz, 1H), 6.56-6.69 (m, 1H), 6.37 (s, 1H), 4.66 (s, 2H), 4.37 (d, J=13.5 Hz, 2H), 3.25 (t, J=11.3 Hz, 2H), 3.20 (t, J=5.9 Hz, 2H), 2.22 (s, 6 H), 1.88-2.03 (m, 4H), 1.83 (m, 2H), 1.53 (d, J=13.3 Hz, 2H); LCMS Rt_{c}=3.19 min, [M+H]⁺=431.2].

### Method L

### Example 16

### 1-(2-(2H-1,2,3-triazol-2-yl)benzyl)-9-(4-methylpyrimidin-2-yl)-1,9-diazaspiro[5.5]undecan-2-one

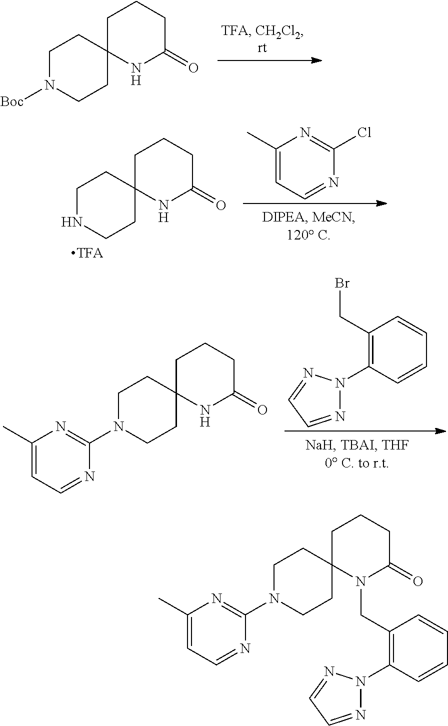

### a) 1,9-diazaspiro[5.5]undecan-2-one (TFA salt)

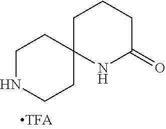 [SMILES: O=C1CCCC2(CCNCC2)N1]

To a solution of tert-butyl 2-oxo-1,9-diazaspiro[5.5]undecane-9-carboxylate (920 mg, 3.26 mmol) in dichloromethane (10 mL) was added TFA (2.53 mL, 32.6 mmol). The solution was stirred for 40 min at rt. After completion of the reaction the mixture was evaporated under reduced pressure and dried under high vacuum (1.90 g, 100%). [¹H NMR (400 MHz, DMSO-d₆) δ ppm 8.59-8.35 (m, 2H), 7.83 (s, 1H), 3.27-3.14 (m, 2H), 3.12-2.98 (m, 2H), 2.17-2.04 (m, 2H), 1.80-1.57 (m, 8 H); LCMS Rt_{A}=0.20, [M+H]⁺=169.2].

### b) 9-(4-methylpyrimidin-2-yl)-1,9-diazaspiro[5.5]undecan-2-one

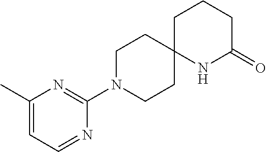 [SMILES: Cc1ccnc(N2CCC3(CCCC(=O)N3)CC2)n1]

To a stirred solution of 1,9-diazaspiro[5.5]undecan-2-one (TFA salt) (1.85 g, 3.17 mmol) and diisopropylethylamine (3.39 mL, 19.05 mmol) in acetonitrile (10 mL) was added 2-chloro-4-methylpyrimidine (600 mg, 4.57 mmol). The mixture was heated at 120° C. in the microwave for 90 min. The reaction mixture was cooled to rt and concentrated under reduced pressure. The crude mixture was purified by flash-column chromatography over silicagel (eluent: dichloromethane/methanol 95:5) to yield the title compound (701 mg, 83%). [¹H NMR (400 MHz, DMSO-d₆). δ ppm 8.17 (d, J=5.02 Hz, 1H), 7.50 (s, 1H), 6.46 (d, J=4.77 Hz, 1H), 4.18-4.08 (m, 2H), 3.47-3.38 (m, 2H), 2.24 (s, 3H), 2.14-2.05 (m, 2H), 1.74-1.49 (m, 8 H); LCMS Rt_{A}=0.71, [M+H]⁺=261.3].

### c) 1-(2-(2H-1,2,3-triazol-2-yl)benzyl)-9-(4-methylpyrimidin-2-yl)-1,9-diazaspiro[5.5]undecan-2-one

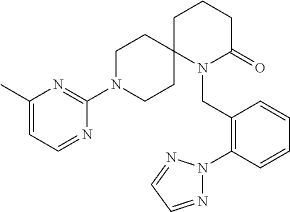 [SMILES: Cc1ccnc(N2CCC3(CCCC(=O)N3Cc3ccccc3-n3nccn3)CC2)n1]

NaH (14 mg, 0.352 mmol, 60% in mineral oil) was added to an ice-cold solution of 9-(4-methylpyrimidin-2-yl)-1,9-diazaspiro[5.5]undecan-2-one (50 mg, 0.19 mmol), 2-(2-(bromomethyl)phenyl)-2H-1,2,3-triazole (described separately as building block) (48 mg, 0.20 mmol) and TBAI (7 mg, 0.019 mmol) in THF (1.5 mL). The resulting mixture was stirred at 0° C. for 1 h. The reaction mixture was allowed to warm to rt and stirred for 6 h. To the mixture water was added and the solution was extracted twice with ethyl acetate. The organic layer was washed with water and brine, filtered and dried over anhydrous sodium sulfate. The organic layer was concentrated under reduced pressure. The crude mixture was purified by flash-column chromatography over silicagel (eluent: gradient 25%-100% ethyl acetate/heptane) to yield the title compound (5 mg, 6%). [¹H NMR (400 MHz, DMSO-d₆) δ ppm 8.15 (d, J=4.77 Hz, 1H), 7.98 (s, 2H), 7.54 (d, J=9.03 Hz, 1H), 7.47-7.37 (m, 2H), 7.22 (d, J=7.03 Hz, 1H), 6.49 (d, J=5.02 Hz, 1H), 4.53-4.40 (m, 4H), 2.97-2.85 (m, 2H), 2.45-2.37 (m, 2H), 2.14-2.06 (m, 2H), 1.83-1.73 (m, 2H), 1.62-1.48 (m, 4H); LCMS Rt_{A}=1.02, [M+H]⁺=418.4].

### Method M

### Example 17

### 2-((1H-indol-3-yl)methyl)-9-(5-methyl-1,3,4-oxadiazol-2-yl)-2,9-diazaspiro[5.5]undecan-1-one

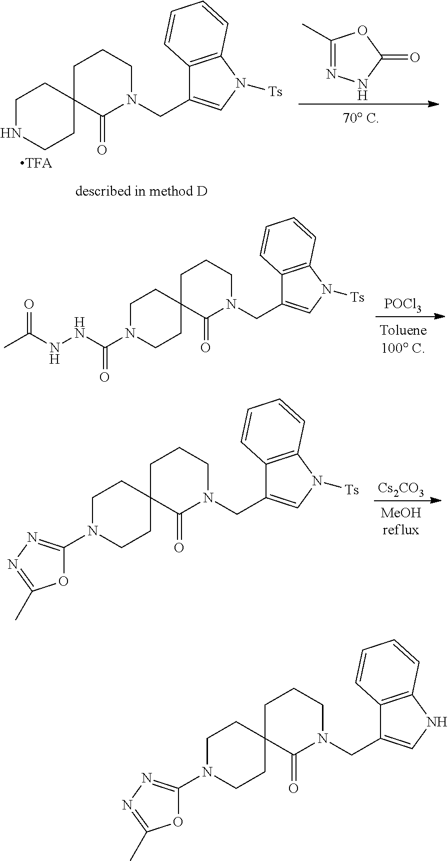

### a) N′-acetyl-1-oxo-2-((1-tosyl-1H-indol-3-yl)methyl)-2,9-diazaspiro[5.5]undecane-9-carbohydrazide

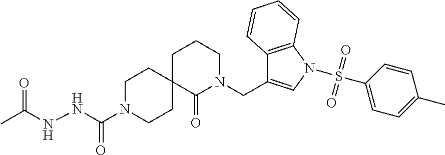 [SMILES: CC(=O)NNC(=O)N1CCC2(CCCN(Cc3cn(S(=O)(=O)c4ccc(C)cc4)c4ccccc34)C2=O)CC1]

The solution of 2-((1-tosyl-1H-indol-3-yl)methyl)-2,9-diazaspiro[5.5]undecan-1-one (TFA salt) (100 mg, 0.155 mmol, prepared according to method D) and 5-methyl-1,3,4-oxadiazol-2-(3H)-one (180 mg, 0.54 mmol) in THF (0.22 ml) was stirred under reflux for 5 h. Thereafter 5-methyl-1,3,4-oxadiazol-2(3H)-one was added again (23 mg, 0.23 mmol) and the mixture stirred for 65 h. The product was purified by flash-column chromatography over silicagel (eluent: gradient 0%-10% DCM/methanol) to yield the title compound (55 mg, 64%). LCMS Rt_{D}=2.52 min, [M+H]⁺=552.2].

### b) 9-(5-methyl-1,3,4-oxadiazol-2-yl)-2-((1-tosyl-1H-indol-3-yl)methyl)-2,9-diazaspiro[5.5]undecan-1-one

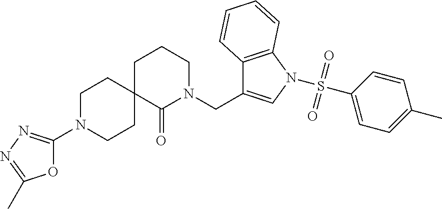 [SMILES: Cc1ccc(S(=O)(=O)n2cc(CN3CCCC4(CCN(c5nnc(C)o5)CC4)C3=O)c3ccccc32)cc1]

The mixture of N′-acetyl-1-oxo-2-((1-tosyl-1H-indol-3-yl)methyl)-2,9-diazaspiro[5.5]undecane-9-carbohydrazide (48 mg, 0.087 mmol) and POCl₃ (40.5 μl, 0.435 mmol) in toluene (1 ml) was stirred at 100° C. overnight. The reaction mixture was poured onto ice and the mixture quenched with saturated aqueous Na₂CO₃ solution. The mixture was extracted with ethyl acetate and the organic layer was washed with water and brine, and dried over anhydrous sodium sulfate. The organic layer was filtered and concentrated under reduced pressure to give 33 mg (71%) of the title compound as a colorless oil. [LCMS Rt_{c}=3.61, [M+H]⁺=534.2].

### c) 2-((1H-indol-3-yl)methyl)-9-(5-methyl-1,3,4-oxadiazol-2-yl)-2,9-diazaspiro[5.5]undecan-1-one

Detosylation of 9-(5-methyl-1,3,4-oxadiazol-2-yl)-2-((1-tosyl-1H-indol-3-yl)methyl)-2,9-diazaspiro[5.5]undecan-1-one was performed according to method D d). The title compound was obtained after chromatography (DCM/ethyl acetate 15:85) on silica gel as a white solid. [¹H NMR (600 MHz, DMSO-d₆) δ ppm 10.98 (br. s., 1H), 7.53 (d, J=8.1 Hz, 1H), 7.35 (d, J=8.1 Hz, 1H), 7.29 (s, 1H), 7.07 (t, J=7.6 Hz, 1H), 6.96 (t, J=7.5 Hz, 1H), 4.63 (s, 2H), 3.61-3.71 (m, 2H), 3.24 (t, J=11.7 Hz, 2H), 3.16 (t, J=5.7 Hz, 2H), 2.34 (s, 3H), 2.06 (s, 2 H), 1.69-1.81 (m, 4H), 1.59-1.69 (m, 2H), 1.47 (d, J=12.9 Hz, 2H); LCMS Rt_{D}=1.57, [M+H]⁺=380.2].

### Method N

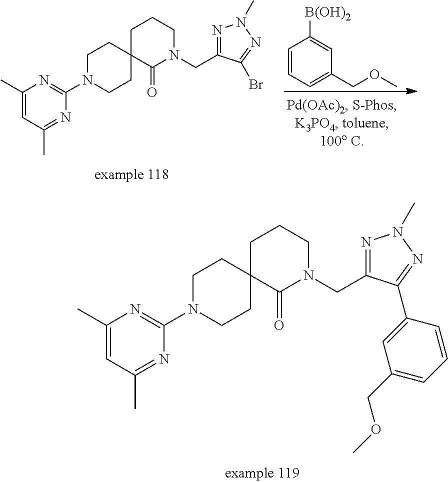

### Example 118

### 2-((5-bromo-2-methyl-2H-1,2,3-triazol-4-yl)methyl)-9-(4,6-dimethylpyrimidin-2-yl)-2,9-diazaspiro[5.5]undecan-1-one

The title compound was obtained as described in method B from tert-butyl 1-oxo-2,9-diazaspiro[5.5]undecane-9-carboxylate and 4-bromo-5-(bromomethyl)-2-methyl-2H-1,2,3-triazole in 3 steps. [¹H NMR (400 MHz, DMSO-d₆) δ ppm 6.35 (s, 1H), 4.52 (s, 2H), 4.30 (dt, J=13.3, 4.1 Hz, 2H), 4.10 (s, 3H), 3.14-3.28 (m, 4H), 2.20 (s, 6 H), 1.70-1.95 (m, 6 H), 1.43 (d, J=13.6 Hz, 2H); LCMS Rt_{A}=1.02 min, [M+H]⁺=448.2/450.2].

### Example 119

### 9-(4,6-dimethylpyrimidin-2-yl)-2-((5-(3-(methoxymethyl)phenyl)-2-methyl-2H-1,2,3-triazol-4-yl)methyl)-2,9-diazaspiro[5.5]undecan-1-one

To a solution of 2-((5-bromo-2-methyl-2H-1,2,3-triazol-4-yl)methyl)-9-(4,6-dimethylpyrimidin-2-yl)-2,9-diazaspiro[5.5]undecan-1-one (157 mg, 0.34 mmol) in toluene (3 ml) was added S-Phos (45.1 mg, 0.11 mmol), (3-(methoxymethyl)phenyl)boronic acid (114 mg, 0.69 mmol), K₃PO₄ (152 mg, 1.09 mmol) and Pd(OAc)₂ (11.6 mg, 0.05 mmol). The reaction vessel was sealed and the mixture was heated at 100° C. for 14 h under argon. Then, the reaction mixture was allowed to warm to room temperature and filtered through a pad of celite. The residue was concentrated under reduced pressure and the crude product was purified by chromatography on silica (Biotage Isolera Four, heptane/EtOAc 90/10 for 2 min, to heptane/EtOAc 0/100 in 13 min and heptane/EtOAc 0/100 for 15 min). The product was crystallized from diisopropylether to give the title compound as white crystals (120 mg, 70%). [¹H NMR (400 MHz, DMSO-d₆) δ ppm 7.49-7.61 (m, 2H), 7.35-7.43 (m, 1H), 7.28-7.35 (m, 1H), 6.35 (s, 1H), 4.75 (s, 2H), 4.43 (s, 2H), 4.22-4.34 (m, 2H), 4.15 (s, 3H), 3.29 (s, 3H), 3.13-3.24 (m, 4H), 2.20 (s, 6 H), 1.79-1.90 (m, 2H), 1.63-1.78 (m, 4H), 1.27-1.41 (m, 2H); LCMS Rt_{A}=1.16 min; 1M+H]⁺=490.4].

TABLE 1
Compounds of Formula (I)
Examples (Ex) 18-127 were synthesized according to respective synthetic methods (SM) A
to N. LCMS: LCMS Rt, [min], (method)

| Ex. | Structure | Name | SM | LCMS | [M + H]⁺ |
|---|---|---|---|---|---|
| 18 | 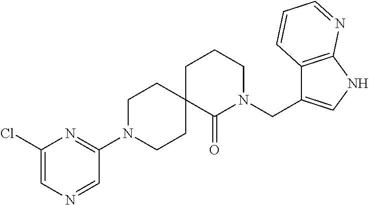 [SMILES: O=C1N(Cc2c[nH]c3ncccc23)CCCC12CCN(c1cncc(Cl)n1)CC2] | 2-((1H-pyrrolo[2,3-b]pyridin-3- yl)methyl)-9-(6-chloropyrazin- 2-yl)-2,9- diazaspiro[5.5]undecan-1-one | A | 0.77 (F) | 411.1 |
| 19 | 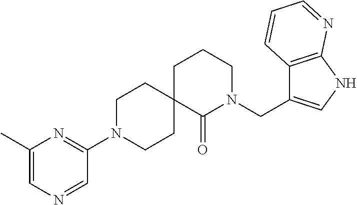 [SMILES: Cc1cncc(N2CCC3(CCCN(Cc4c[nH]c5ncccc45)C3=O)CC2)n1] | 2-((1H-pyrrolo[2,3-b]pyridin-3- yl)methyl)-9-(6-methylpyrazin- 2-yl)-2,9- diazaspiro[5.5]undecan-1-one | A | 0.23 (E) | 391.2 |
| 20 | 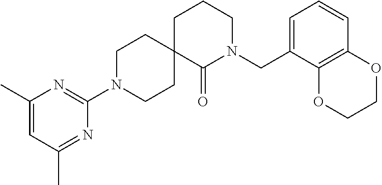 [SMILES: Cc1cc(C)nc(N2CCC3(CCCN(Cc4cccc5c4OCCO5)C3=O)CC2)n1] | 2-((2,3- dihydrobenzo[b][1,4]dioxin-5- yl)methyl)-9-(4,6- dimethylpyrimidin-2-yl)-2,9- diazaspiro[5.5]undecan-1-one | A | 0.82 (F) | 423.1 |
| 21 | 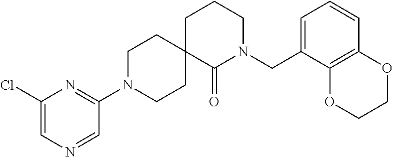 [SMILES: O=C1N(Cc2cccc3c2OCCO3)CCCC12CCN(c1cncc(Cl)n1)CC2] | 9-(6-chloropyrazin-2-yl)-2- ((2,3- dihydrobenzo[b][1,4]dioxin-5- yl)methyl)-2,9- diazaspiro[5.5]undecan-1-one | A | 1.69 (E) | 429.1 |
| 22 | 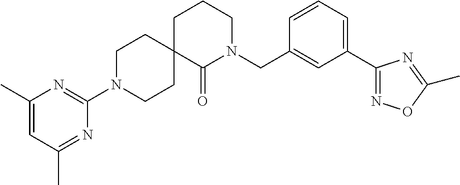 | 9-(4,6-dimethylpyrimidin-2-yl)- 2-(3-(5-methyl-1,2,4-oxadiazol- 3-yl)benzyl)-2,9- diazaspiro[5.5]undecan-1-one | A | 2.94 (C) | 447.2 |
| 23 | 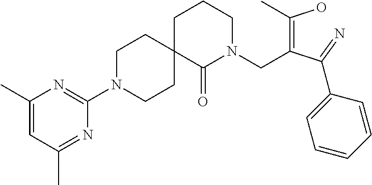 [SMILES: C/C(O)=C(\CN1CCCC2(CCN(c3nc(C)cc(C)n3)CC2)C1=O)C(=N)c1ccccc1] | 9-(4,6-dimethylpyrimidin-2-yl)- 2-((5-methyl-3-phenylisoxazol- 4-yl)methyl)-2,9- diazaspiro[5.5]undecan-1-one | A | 2.98 (C) | 446.2 |
| 24 | 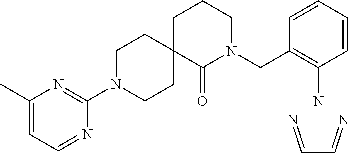 | 2-(2-(2H-1,2,3-triazol-2- yl)benzyl)-9-(4- methylpyrimidin-2-yl)-2,9- diazaspiro[5.5]undecan-1-one | B | 1.10 (A) | 418.4 |
| 25 | 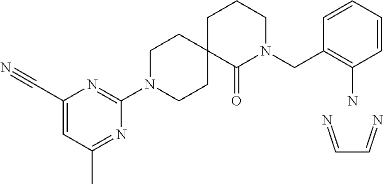 | 2-(2-(2-(2H-1,2,3-triazol-2- yl)benzyl)-1-oxo-2,9- diazaspiro[5.5]undecan-9-yl)-6- methylpyrimidine-4-carbonitrile | B | 1.24 (A) | 443.4 |
| 26 | 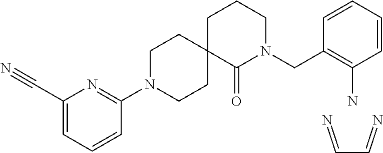 | 6-(2-(2-(2H-1,2,3-triazol-2- yl)benzyl)-1-oxo-2,9- diazaspiro[5.5]undecan-9- yl)picolinonitrile | B | 1.17 (A) | 428.4 |
| 27 | 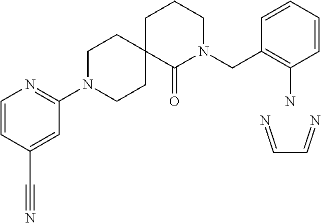 | 2-(2-(2-(2H-1,2,3-triazol-2- yl)benzyl)-1-oxo-2,9- diazaspiro[5.5]undecan-9- yl)isonicotinonitrile | B | 1.14 (A) | 428.4 |
| 28 | 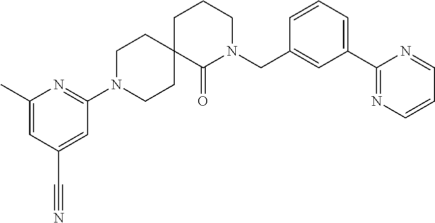 [SMILES: Cc1cc(C#N)cc(N2CCC3(CCCN(Cc4cccc(-c5ncccn5)c4)C3=O)CC2)n1] | 2-methyl-6-(1-oxo-2-(3- (pyrimidin-2-yl)benzyl)-2,9- diazaspiro[5.5]undecan-9- yl)isonicotinonitrile | B | 1.26 (A) | 453.4 |
| 29 | 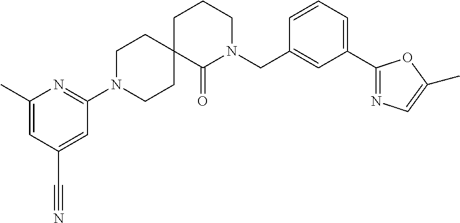 | 2-methyl-6-(2-(3-(5- methyloxazol-2-yl)benzyl)-1- oxo-2,9- diazaspiro[5.5]undecan-9- yl)isonicotinonitrile | B | 1.30 (A) | 453.4 |
| 30 | 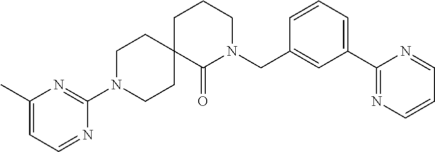 [SMILES: Cc1ccnc(N2CCC3(CCCN(Cc4cccc(-c5ncccn5)c4)C3=O)CC2)n1] | 9-(4-methylpyrimidin-2-yl)-2-(3- (pyrimidin-2-yl)benzyl)-2,9- diazaspiro[5.5]undecan-1-one | B | 1.18 (A) | 429.4 |
| 31 | 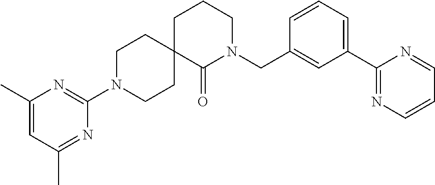 [SMILES: Cc1cc(C)nc(N2CCC3(CCCN(Cc4cccc(-c5ncccn5)c4)C3=O)CC2)n1] | 9-(4,6-dimethylpyrimidin-2-yl)- 2-(3-(pyrimidin-2-yl)benzyl)- 2,9-diazaspiro[5.5]undecan-1- one | B | 1.15 (A) | 443.4 |
| 32 | 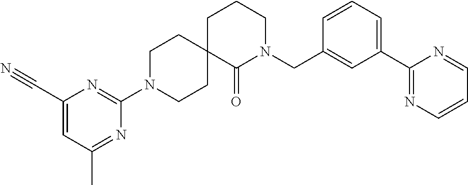 [SMILES: Cc1cc(C#N)nc(N2CCC3(CCCN(Cc4cccc(-c5ncccn5)c4)C3=O)CC2)n1] | 6-methyl-2-(1-oxo-2-(3- (pyrimidin-2-yl)benzyl)-2,9- diazaspiro[5.5]undecan-9- yl)pyrimidine-4-carbonitrile | B | 1.25 (A) | 454.4 |
| 33 | 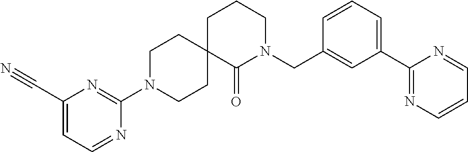 [SMILES: N#Cc1ccnc(N2CCC3(CCCN(Cc4cccc(-c5ncccn5)c4)C3=O)CC2)n1] | 2-(1-oxo-2-(3-(pyrimidin-2- yl)benzyl)-2,9- diazaspiro[5.5]undecan-9- yl)pyrimidine-4-carbonitrile | B | 1.17 (A) | 440.4 |
| 34 | 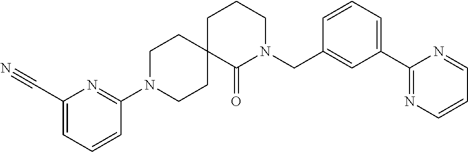 [SMILES: N#Cc1cccc(N2CCC3(CCCN(Cc4cccc(-c5ncccn5)c4)C3=O)CC2)n1] | 6-(1-oxo-2-(3-(pyrimidin-2- yl)benzyl)-2,9- diazaspiro[5.5]undecan-9- yl)picolinonitrile | B | 1.17 A) | 439.4 |
| 35 | 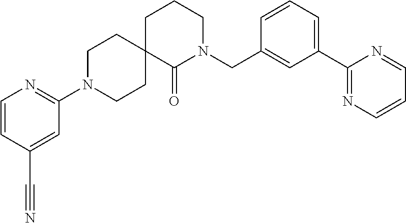 [SMILES: N#Cc1ccnc(N2CCC3(CCCN(Cc4cccc(-c5ncccn5)c4)C3=O)CC2)c1] | 2-(1-oxo-2-(3-(pyrimidin-2- yl)benzyl)-2,9- diazaspiro[5.5]undecan-9- yl)isonicotinonitrile | B | 1.15 (A) | 439.4 |
| 36 | 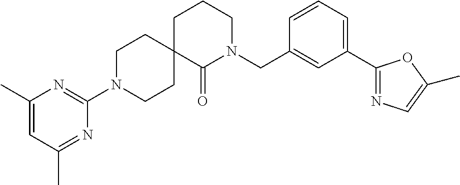 | 9-(4,6-dimethylpyrimidin-2-yl)- 2-(3-(5-methyloxazol-2- yl)benzyl)-2,9- diazaspiro[5.5]undecan-1-one | B | 1.21 (A) | 446.4 |
| 37 | 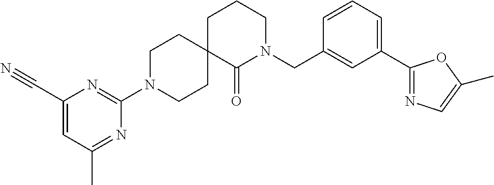 [SMILES: Cc1cc(C#N)nc(N2CCC3(CCCN(Cc4cccc(-c5ncc(C)o5)c4)C3=O)CC2)n1] | 6-methyl-2-(2-(3-(5- methyloxazol-2-yl)benzyl)-1- oxo-2,9- diazaspiro[5.5]undecan-9- yl)pyrimidine-4-carbonitrile | B | 1.29 (A) | 457.4 |
| 38 | 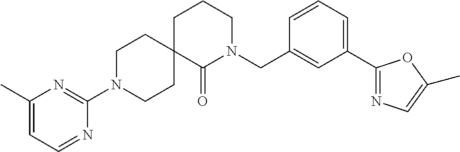 | 2-(3-(5-methyloxazol-2- yl)benzyl)-9-(4- methylpyrimidin-2-yl)-2,9- diazaspiro[5.5]undecan-1-one | B | 1.16 (A) | 432.4 |
| 39 | 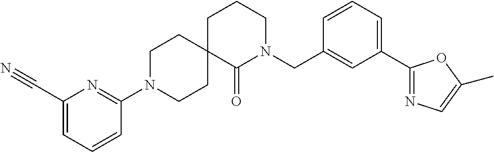 [SMILES: Cc1cnc(-c2cccc(CN3CCCC4(CCN(c5cccc(C#N)n5)CC4)C3=O)c2)o1] | 6-(2-(3-(5-methyloxazol-2- yl)benzyl)-1-oxo-2,9- diazaspiro[5.5]undecan-9- yl)picolinonitrile | B | 1.21 (A) | 442.4 |
| 40 | 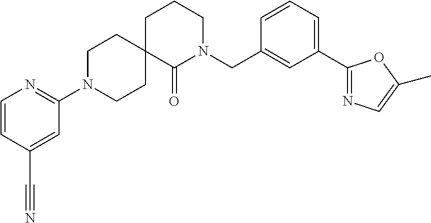 [SMILES: Cc1cnc(-c2cccc(CN3CCCC4(CCN(c5cc(C#N)ccn5)CC4)C3=O)c2)o1] | 2-(2-(3-(5-methyloxazol-2- yl)benzyl)-1-oxo-2,9- diazaspiro[5.5]undecan-9- yl)isonicotinonitrile | B | 1.19 (A) | 442.4 |
| 41 | 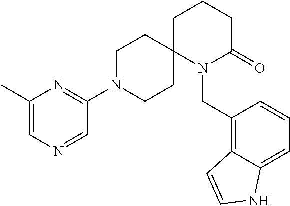 [SMILES: Cc1cncc(N2CCC3(CCCC(=O)N3Cc3cccc4[nH]ccc34)CC2)n1] | 1-((1H-indol-4-yl)methyl)-9-(6- methylpyrazin-2-yl)-1,9- diazaspiro[5.5]undecan-2-one | C | 0.85 (F) | 390.2 |
| 42 | 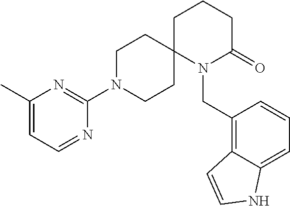 [SMILES: Cc1ccnc(N2CCC3(CCCC(=O)N3Cc3cccc4[nH]ccc34)CC2)n1] | 1-((1H-indol-4-yl)methyl)-9-(4- methylpyrimidin-2-yl)-1,9- diazaspiro[5.5]undecan-2-one | C | 0.95 (A) | 390.3 |
| 43 | 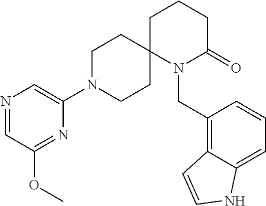 [SMILES: COc1cncc(N2CCC3(CCCC(=O)N3Cc3cccc4[nH]ccc34)CC2)n1] | 1-((1H-indol-4-yl)methyl)-9-(6- methoxypyrazin-2-yl)-1,9- diazaspiro[5.5]undecan-2-one | C | 1.15 (F) | 406.1 |
| 44 | 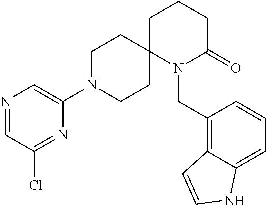 [SMILES: O=C1CCCC2(CCN(c3cncc(Cl)n3)CC2)N1Cc1cccc2[nH]ccc12] | 1-((1H-indol-4-yl)methyl)-9-(6- chloropyrazin-2-yl)-1,9- diazaspiro[5.5]undecan-2-one | C | 1.44 (F) | 410.2 |
| 45 | 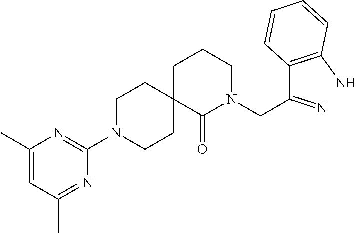 [SMILES: Cc1cc(C)nc(N2CCC3(CCCN(CC(=N)c4ccccc4N)C3=O)CC2)n1] | 2-((1H-indazol-3-yl)methyl)-9- (4,6-dimethylpyrimidin-2-yl)- 2,9-diazaspiro[5.5]undecan-1- one | D | 1.00 (A) | 405.4 |
| 46 | 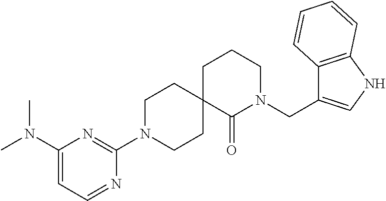 [SMILES: CN(C)c1ccnc(N2CCC3(CCCN(Cc4c[nH]c5ccccc45)C3=O)CC2)n1] | 2-((1H-indol-3-yl)methyl)-9-(4- (dimethylamino)pyrimidin-2-yl)- 2,9-diazaspiro[5.5]undecan-1- one | D | 0.81 (A) | 419.4 |
| 47 | 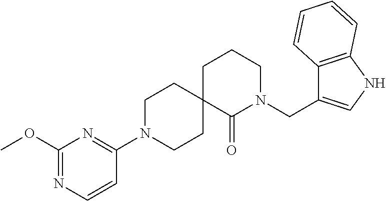 [SMILES: COc1nccc(N2CCC3(CCCN(Cc4c[nH]c5ccccc45)C3=O)CC2)n1] | 2-((1H-indol-3-yl)methyl)-9-(2- methoxypyrimidin-4-yl)-2,9- diazaspiro[5.5]undecan-1-one | D | 0.77 (A) | 406.3 |
| 48 | 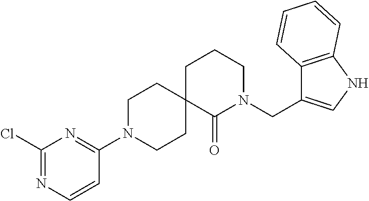 [SMILES: O=C1N(Cc2c[nH]c3ccccc23)CCCC12CCN(c1ccnc(Cl)n1)CC2] | 2-((1H-indol-3-yl)methyl)-9-(2- chloropyrimidin-4-yl)-2,9- diazaspiro[5.5]undecan-1-one | D | 1.02 (A) | 410.3 |
| 49 | 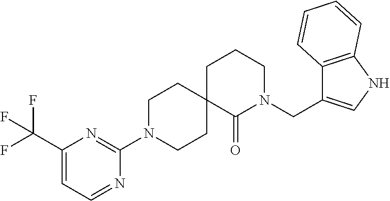 [SMILES: O=C1N(Cc2c[nH]c3ccccc23)CCCC12CCN(c1nccc(C(F)(F)F)n1)CC2] | 2-((1H-indol-3-yl)methyl)-9-(4- (trifluoromethyl)pyrimidin-2-yl)- 2,9-diazaspiro[5.5]undecan-1- one | D | 1.25 (A) | 444.3 |
| 50 | 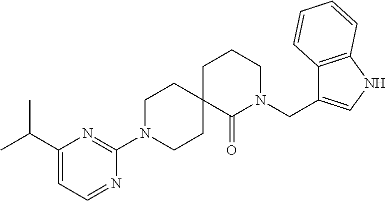 [SMILES: CC(C)c1ccnc(N2CCC3(CCCN(Cc4c[nH]c5ccccc45)C3=O)CC2)n1] | 2-((1H-indol-3-yl)methyl)-9-(4- isopropylpyrimidin-2-yl)-2,9- diazaspiro[5.5]undecan-1-one | D | 1.25 (A) | 418.4 |
| 51 | 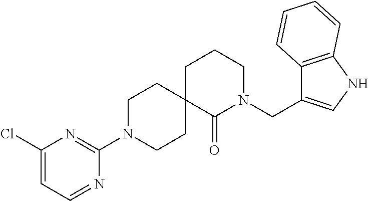 [SMILES: O=C1N(Cc2c[nH]c3ccccc23)CCCC12CCN(c1nccc(Cl)n1)CC2] | 2-((1H-indol-3-yl)methyl)-9-(4- chloropyrimidin-2-yl)-2,9- diazaspiro[5.5]undecan-1-one | D | 1.20 (A) | 410.3 |
| 52 | 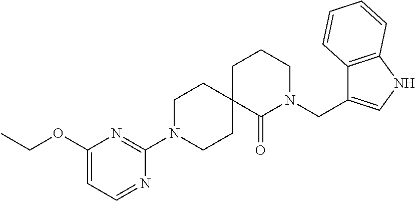 [SMILES: CCOc1ccnc(N2CCC3(CCCN(Cc4c[nH]c5ccccc45)C3=O)CC2)n1] | 2-((1H-indol-3-yl)methyl)-9-(4- ethoxypyrimidin-2-yl)-2,9- diazaspiro[5.5]undecan-1-one | D | 1.03 (A) | 420.4 |
| 53 | 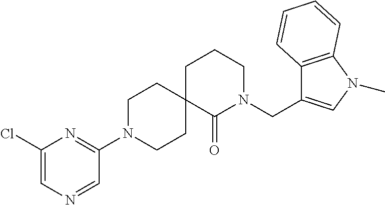 [SMILES: Cn1cc(CN2CCCC3(CCN(c4cncc(Cl)n4)CC3)C2=O)c2ccccc21] | 9-(6-chloropyrazin-2-yl)-2-((1- methyl-1H-indol-3-yl)methyl)- 2,9-diazaspiro[5.5]undecan-1- one | D¹ | 1.08 (F) | 424.2 |
| 54 | 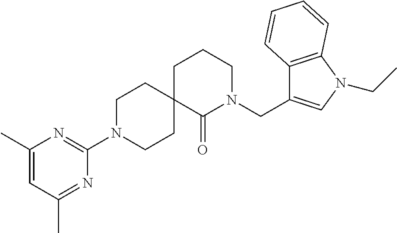 [SMILES: CCn1cc(CN2CCCC3(CCN(c4nc(C)cc(C)n4)CC3)C2=O)c2ccccc21] | 9-(4,6-dimethylpyrimidin-2-yl)- 2-((1-ethyl-1H-indol-3- yl)methyl)-2,9- diazaspiro[5.5]undecan-1-one | D² | 1.75 (F) | 432.2 |
| 55 | 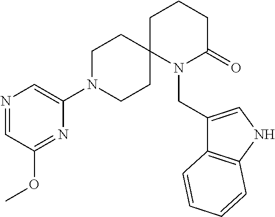 [SMILES: COc1cncc(N2CCC3(CCCC(=O)N3Cc3c[nH]c4ccccc34)CC2)n1] | 1-((1H-indol-3-yl)methyl)-9-(6- methoxypyrazin-2-yl)-1,9- diazaspiro[5.5]undecan-2-one | E | 1.73 (F) | 406.0 |
| 56 | 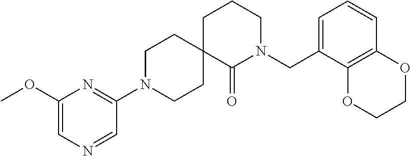 [SMILES: COc1cncc(N2CCC3(CCCN(Cc4cccc5c4OCCO5)C3=O)CC2)n1] | 2-((2,3- dihydrobenzo[b][1,4]dioxin-5- yl)methyl)-9-(6- methoxypyrazin-2-yl)-2,9- diazaspiro[5.5]undecan-1-one | F | 1.57 (F) | 425.4 |
| 57 | 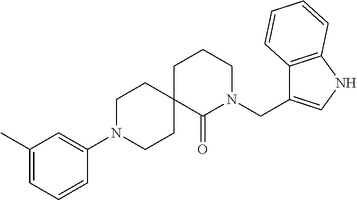 [SMILES: Cc1cccc(N2CCC3(CCCN(Cc4c[nH]c5ccccc45)C3=O)CC2)c1] | 2-((1H-indol-3-yl)methyl)-9-m- tolyl-2,9- diazaspiro[5.5]undecan-1-one | G | 1.11 (A) | 388.3 |
| 58 | 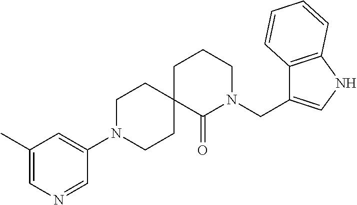 [SMILES: Cc1cncc(N2CCC3(CCCN(Cc4c[nH]c5ccccc45)C3=O)CC2)c1] | 2-((1H-indol-3-yl)methyl)-9-(5- methylpyridin-3-yl)-2,9- diazaspiro[5.5]undecan-1-one | G | 0.76 (A) | 389.3 |
| 59 | 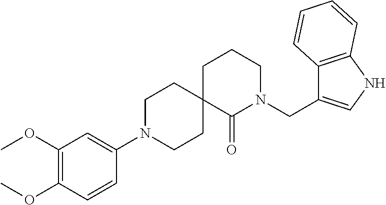 [SMILES: COc1ccc(N2CCC3(CCCN(Cc4c[nH]c5ccccc45)C3=O)CC2)cc1OC] | 2-((1H-indol-3-yl)methyl)-9- (3,4-dimethoxyphenyl)-2,9- diazaspiro[5.5]undecan-1-one | G | 0.88 (A) | 434.4 |
| 60 | 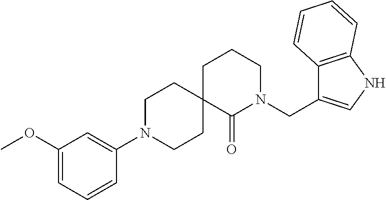 [SMILES: COc1cccc(N2CCC3(CCCN(Cc4c[nH]c5ccccc45)C3=O)CC2)c1] | 2-((1H-indol-3-yl)methyl)-9-(3- methoxyphenyl)-2,9- diazaspiro[5.5]undecan-1-one | G | 1.11 (A) | 404.4 |
| 61 | 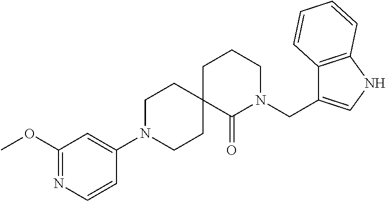 [SMILES: COc1cc(N2CCC3(CCCN(Cc4c[nH]c5ccccc45)C3=O)CC2)ccn1] | 2-((1H-indol-3-yl)methyl)-9-(2- methoxypyridin-4-yl)-2,9- diazaspiro[5.5]undecan-1-one | G | 0.74 (A) | 405.4 |
| 62 | 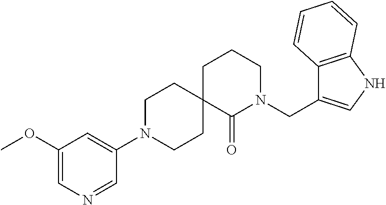 [SMILES: COc1cncc(N2CCC3(CCCN(Cc4c[nH]c5ccccc45)C3=O)CC2)c1] | 2-((1H-indol-3-yl)methyl)-9-(5- methoxypyridin-3-yl)-2,9- diazaspiro[5.5]undecan-1-one | G | 0.81 (A) | 405.0 |
| 63 | 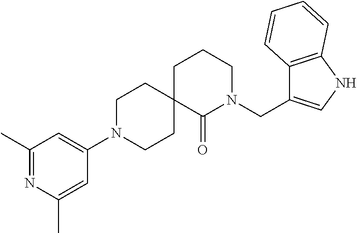 [SMILES: Cc1cc(N2CCC3(CCCN(Cc4c[nH]c5ccccc45)C3=O)CC2)cc(C)n1] | 2-((1H-indol-3-yl)methyl)-9- (2,6-dimethylpyridin-4-yl)-2,9- diazaspiro[5.5]undecan-1-one | G | 0.77 (A) | 403.4 |
| 64 | 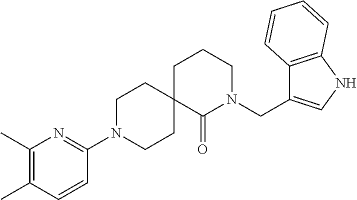 [SMILES: Cc1ccc(N2CCC3(CCCN(Cc4c[nH]c5ccccc45)C3=O)CC2)nc1C] | 2-((1H-indol-3-yl)methyl)-9- (5,6-dimethylpyridin-2-yl)-2,9- diazaspiro[5.5]undecan-1-one | G | 0.82 (A) | 403.2 |
| 65 | 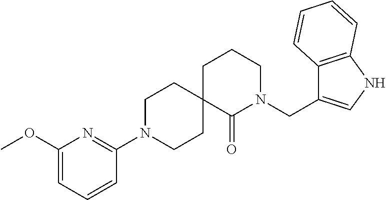 [SMILES: COc1cccc(N2CCC3(CCCN(Cc4c[nH]c5ccccc45)C3=O)CC2)n1] | 2-((1H-indol-3-yl)methyl)-9-(6- methoxypyridin-2-yl)-2,9- diazaspiro[5.5]undecan-1-one | G | 3.33 (C) | 405.2 |
| 66 | 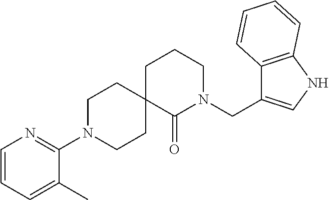 [SMILES: Cc1cccnc1N1CCC2(CCCN(Cc3c[nH]c4ccccc34)C2=O)CC1] | 2-((1H-indol-3-yl)methyl)-9-(3- methylpyridin-2-yl)-2,9- diazaspiro[5.5]undecan-1-one | G | 2.86 (C) | 389.2 |
| 67 | 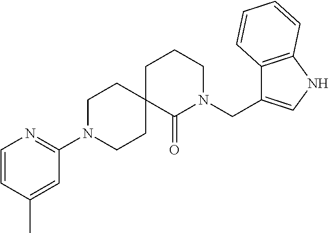 [SMILES: Cc1ccnc(N2CCC3(CCCN(Cc4c[nH]c5ccccc45)C3=O)CC2)c1] | 2-((1H-indol-3-yl)methyl)-9-(4- methylpyridin-2-yl)-2,9- diazaspiro[5.5]undecan-1-one | G | 2.86 (C) | 389.2 |
| 68 | 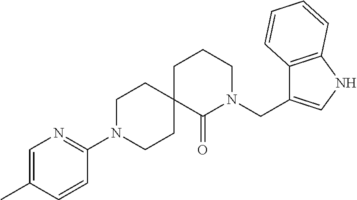 [SMILES: Cc1ccc(N2CCC3(CCCN(Cc4c[nH]c5ccccc45)C3=O)CC2)nc1] | 2-((1H-indol-3-yl)methyl)-9-(5- methylpyridin-2-yl)-2,9- diazaspiro[5.5]undecan-1-one | G | 2.84 (C) | 389.2 |
| 69 | 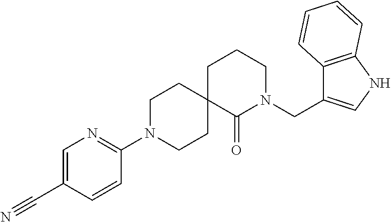 [SMILES: N#Cc1ccc(N2CCC3(CCCN(Cc4c[nH]c5ccccc45)C3=O)CC2)nc1] | 6-(2-((1H-indol-3-yl)methyl)-1- oxo-2,9- diazaspiro[5.5]undecan-9- yl)nicotinonitrile | G | 2.71 (C) | 400.2 |
| 70 | 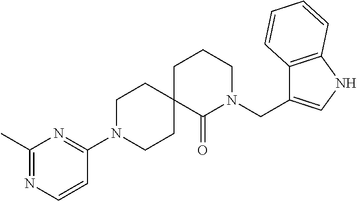 [SMILES: Cc1nccc(N2CCC3(CCCN(Cc4c[nH]c5ccccc45)C3=O)CC2)n1] | 2-((1H-indol-3-yl)methyl)-9-(2- methylpyrimidin-4-yl)-2,9- diazaspiro[5.5]undecan-1-one | G | 2.80 (C) | 390.2 |
| 71 | 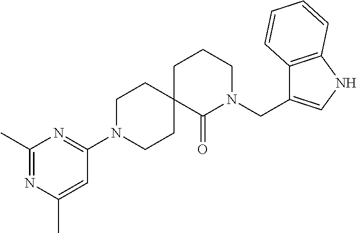 [SMILES: Cc1cc(N2CCC3(CCCN(Cc4c[nH]c5ccccc45)C3=O)CC2)nc(C)n1] | 2-((1H-indol-3-yl)methyl)-9- (2,6-dimethylpyrimidin-4-yl)- 2,9-diazaspiro[5.5]undecan-1- one | G | 1.26 (D) | 404.2 |
| 72 | 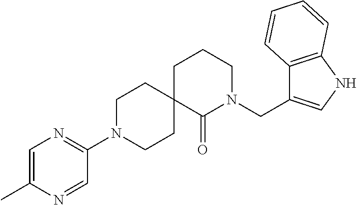 [SMILES: Cc1cnc(N2CCC3(CCCN(Cc4c[nH]c5ccccc45)C3=O)CC2)cn1] | 2-((1H-indol-3-yl)methyl)-9-(5- methylpyrazin-2-yl)-2,9- diazaspiro[5.5]undecan-1-one | G | 1.57 (D) | 390.2 |
| 73 | 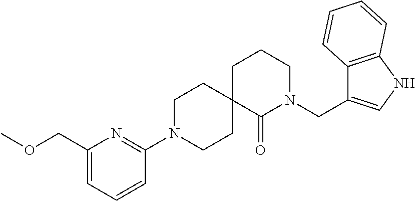 [SMILES: COCc1cccc(N2CCC3(CCCN(Cc4c[nH]c5ccccc45)C3=O)CC2)n1] | 2-((1H-indol-3-yl)methyl)-9-(6- (methoxymethyl)pyridin-2-yl)- 2,9-diazaspiro[5.5]undecan-1- one | G | 2.80 (C) | 419.2 |
| 74 | 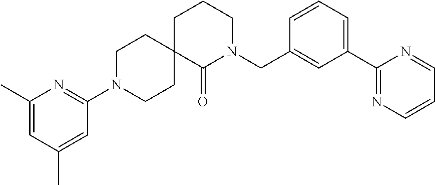 [SMILES: Cc1cc(C)nc(N2CCC3(CCCN(Cc4cccc(-c5ncccn5)c4)C3=O)CC2)c1] | 9-(4,6-dimethylpyridin-2-yl)-2- (3-(pyrimidin-2-yl)benzyl)-2,9- diazaspiro[5.5]undecan-1-one | G | 0.81 (A) | 442.4 |
| 75 | 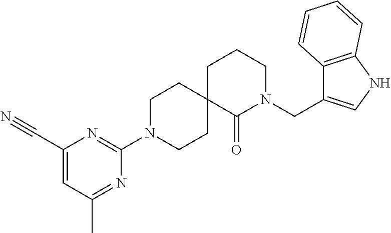 [SMILES: Cc1cc(C#N)nc(N2CCC3(CCCN(Cc4c[nH]c5ccccc45)C3=O)CC2)n1] | 2-(2-((1H-indol-3-yl)methyl)-1- oxo-2,9- diazaspiro[5.5]undecan-9-yl)-6- methylpyrimidine-4-carbonitrile | H | 1.20 (A) | 415.3 |
| 76 | 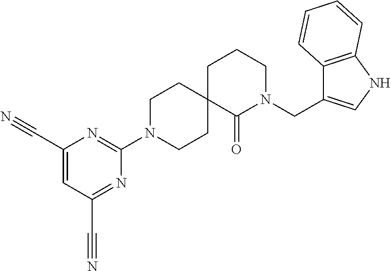 [SMILES: N#Cc1cc(C#N)nc(N2CCC3(CCCN(Cc4c[nH]c5ccccc45)C3=O)CC2)n1] | 2-(2-((1H-indol-3-yl)methyl)-1- oxo-2,9- diazaspiro[5.5]undecan-9- yl)pyrimidine-4,6-dicarbonitrile | H | 1.29 (A) | not found |
| 77 | 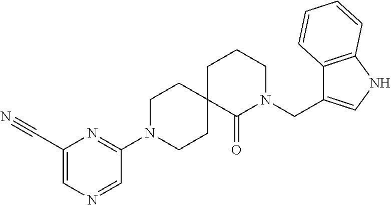 [SMILES: N#Cc1cncc(N2CCC3(CCCN(Cc4c[nH]c5ccccc45)C3=O)CC2)n1] | 6-(2-((1H-indol-3-yl)methyl)-1- oxo-2,9- diazaspiro[5.5]undecan-9- yl)pyrazine-2-carbonitrile | H | 1.07 (A) | 401.3 |
| 78 | 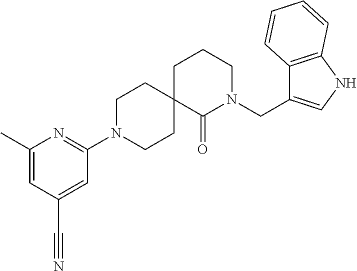 [SMILES: Cc1cc(C#N)cc(N2CCC3(CCCN(Cc4c[nH]c5ccccc45)C3=O)CC2)n1] | 2-(2-((1H-indol-3-yl)methyl)-1- oxo-2,9- diazaspiro[5.5]undecan-9-yl)-6- methylisonicotinonitrile | H | 1.19 (A) | 414.2 |
| 79 | 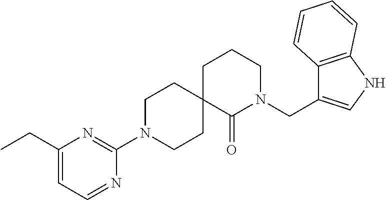 [SMILES: CCc1ccnc(N2CCC3(CCCN(Cc4c[nH]c5ccccc45)C3=O)CC2)n1] | 2-((1H-indol-3-yl)methyl)-9-(4- ethylpyrimidin-2-yl)-2,9- diazaspiro[5.5]undecan-1-one | H | 1.17 (A) | 404.4 |
| 80 | 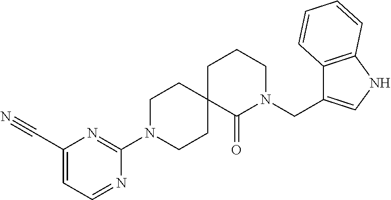 [SMILES: N#Cc1ccnc(N2CCC3(CCCN(Cc4c[nH]c5ccccc45)C3=O)CC2)n1] | 2-(2-((1H-indol-3-yl)methyl)-1- oxo-2,9- diazaspiro[5.5]undecan-9- yl)pyrimidine-4-carbonitrile | H | 1.12 (A) | 401.3 |
| 81 | 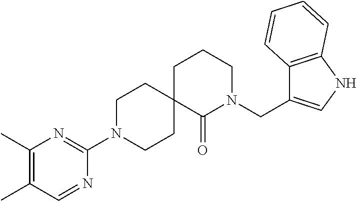 [SMILES: Cc1cnc(N2CCC3(CCCN(Cc4c[nH]c5ccccc45)C3=O)CC2)nc1C] | 2-((1H-indol-3-yl)methyl)-9- (4,5-dimethylpyrimidin-2-yl)- 2,9-diazaspiro[5.5]undecan-1- one | H | 1.11 (A) | 404.4 |
| 82 | 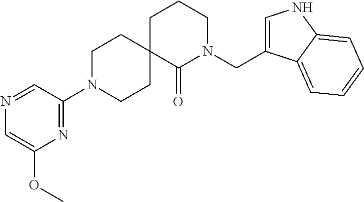 [SMILES: COc1cncc(N2CCC3(CCCN(Cc4c[nH]c5ccccc45)C3=O)CC2)n1] | 2-((1H-indol-3-yl)methyl)-9-(6- methoxypyrazin-2-yl)-2,9- diazaspiro[5.5]undecan-1-one | H | 2.38 (A) | 406.2 |
| 83 | 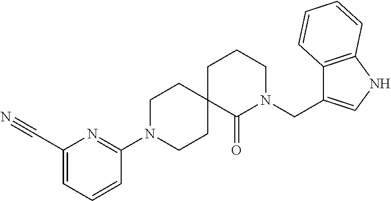 [SMILES: N#Cc1cccc(N2CCC3(CCCN(Cc4c[nH]c5ccccc45)C3=O)CC2)n1] | 6-(2-((1H-indol-3-yl)methyl)-1- oxo-2,9- diazaspiro[5.5]undecan-9- yl)picolinonitrile | H | 3.10 (D) | 400.2 |
| 84 | 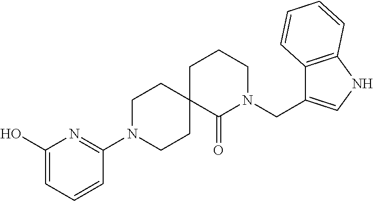 [SMILES: O=C1N(Cc2c[nH]c3ccccc23)CCCC12CCN(c1cccc(O)n1)CC2] | 2-((1H-indol-3-yl)methyl)-9-(6- hydroxypyridin-2-yl)-2,9- diazaspiro[5.5]undecan-1-one | H | 2.84 (C) | 391.2 |
| 85 | 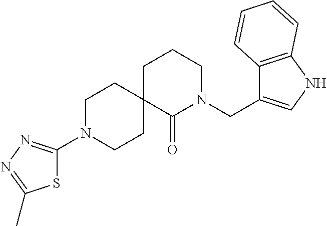 [SMILES: Cc1nnc(N2CCC3(CCCN(Cc4c[nH]c5ccccc45)C3=O)CC2)s1] | 2-((1H-indol-3-yl)methyl)-9-(5- methyl-1,3,4-thiadiazol-2-yl)- 2,9-diazaspiro[5.5]undecan-1- one | H | 1.17 (D) | 396.2 |
| 86 | 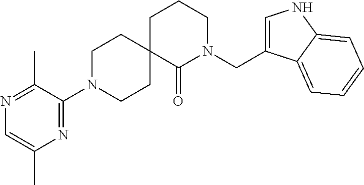 [SMILES: Cc1cnc(C)c(N2CCC3(CCCN(Cc4c[nH]c5ccccc45)C3=O)CC2)n1] | 2-((1H-indol-3-yl)methyl)-9- (3,6-dimethylpyrazin-2-yl)-2,9- diazaspiro[5.5]undecan-1-one | H | 0.98 (G) | 404.0 |
| 87 | 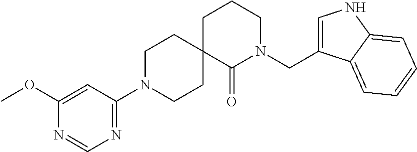 [SMILES: COc1cc(N2CCC3(CCCN(Cc4c[nH]c5ccccc45)C3=O)CC2)ncn1] | 2-((1H-indol-3-yl)methyl)-9-(6- methoxypyrimidin-4-yl)-2,9- diazaspiro[5.5]undecan-1-one | H | 0.82 (G) | 406.0 |
| 88 | 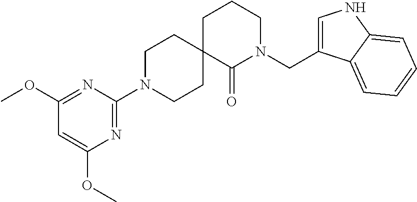 [SMILES: COc1cc(OC)nc(N2CCC3(CCCN(Cc4c[nH]c5ccccc45)C3=O)CC2)n1] | 2-((1H-indol-3-yl)methyl)-9- (4,6-dimethoxypyrimidin-2-yl)- 2,9-diazaspiro[5.5]undecan-1- one | H | 1.25 (G) | 436.0 |
| 89 | 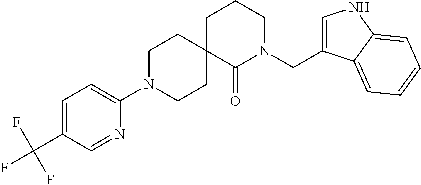 [SMILES: O=C1N(Cc2c[nH]c3ccccc23)CCCC12CCN(c1ccc(C(F)(F)F)cn1)CC2] | 2-((1H-indol-3-yl)methyl)-9-(5- (trifluoromethyl)pyridin-2-yl)- 2,9-diazaspiro[5.5]undecan-1- one | H | 1.17 (G) | 443.0 |
| 90 | 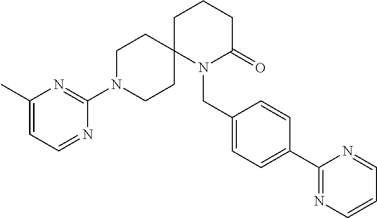 [SMILES: Cc1ccnc(N2CCC3(CCCC(=O)N3Cc3ccc(-c4ncccn4)cc3)CC2)n1] | 9-(4-methylpyrimidin-2-yl)-1-(4- (pyrimidin-2-yl)benzyl)-1,9- diazaspiro[5.5]undecan-2-one | L | 2.63 (C) | 429.2 |
| 91 | 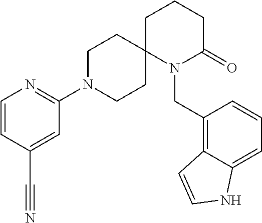 [SMILES: N#Cc1ccnc(N2CCC3(CCCC(=O)N3Cc3cccc4[nH]ccc34)CC2)c1] | 2-(1-((1H-indol-4-yl)methyl)-2- oxo-1,9- diazaspiro[5.5]undecan-9- yl)isonicotinonitrile | I | 0.98 (A) | 400.3 |
| 92 | 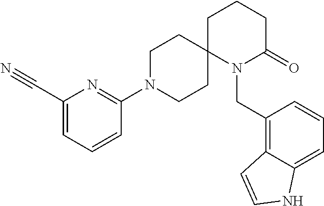 [SMILES: N#Cc1cccc(N2CCC3(CCCC(=O)N3Cc3cccc4[nH]ccc34)CC2)n1] | 6-(1-((1H-indol-4-yl)methyl)-2- oxo-1,9- diazaspiro[5.5]undecan-9- yl)picolinonitrile | I | 0.97 (A) | 400.2 |
| 93 | 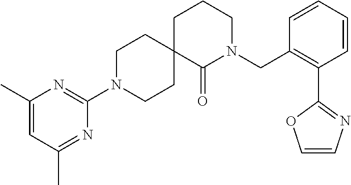 [SMILES: Cc1cc(C)nc(N2CCC3(CCCN(Cc4ccccc4-c4ncco4)C3=O)CC2)n1] | 9-(4,6-dimethylpyrimidin-2-yl)- 2-(2-(oxazol-2-yl)benzyl)-2,9- diazaspiro[5.5]undecan-1-one | K | 2.93 (C) | 432.2 |
| 94 | 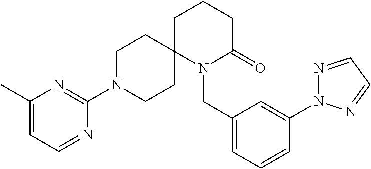 [SMILES: Cc1ccnc(N2CCC3(CCCC(=O)N3Cc3cccc(-n4nccn4)c3)CC2)n1] | 1-(3-(2H-1,2,3-triazol-2- yl)benzyl)-9-(4- methylpyrimidin-2-yl)-1,9- diazaspiro[5.5]undecan-2-one | L | 1.05 (A) | 418.4 |
| 95 | 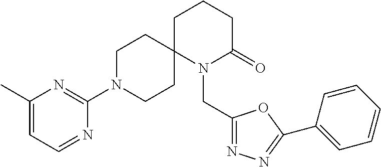 [SMILES: Cc1ccnc(N2CCC3(CCCC(=O)N3Cc3nnc(-c4ccccc4)o3)CC2)n1] | 9-(4-methylpyrimidin-2-yl)-1- ((5-phenyl-1,3,4-oxadiazol-2- yl)methyl)-1,9- diazaspiro[5.5]undecan-2-one | L | 2.66 (C) | 419.2 |
| 96 | 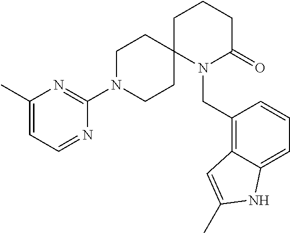 [SMILES: Cc1ccnc(N2CCC3(CCCC(=O)N3Cc3cccc4[nH]c(C)cc34)CC2)n1] | 1-((2-methyl-1H-indol-4- yl)methyl)-9-(4- methylpyrimidin-2-yl)-1,9- diazaspiro[5.5]undecan-2-one | L | 2.70 (C) | 404.2 |
| 97 | 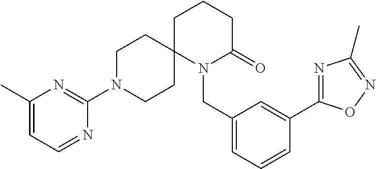 [SMILES: Cc1ccnc(N2CCC3(CCCC(=O)N3Cc3cccc(-c4nc(C)no4)c3)CC2)n1] | 1-(3-(3-methyl-1,2,4-oxadiazol- 5-yl)benzyl)-9-(4- methylpyrimidin-2-yl)-1,9- diazaspiro[5.5]undecan-2-one | L | 2.77 (C) | 433.2 |
| 98 | 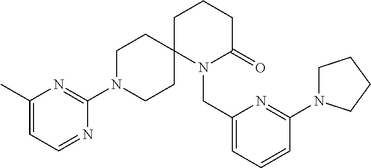 [SMILES: Cc1ccnc(N2CCC3(CCCC(=O)N3Cc3cccc(N4CCCC4)n3)CC2)n1] | 9-(4-methylpyrimidin-2-yl)-1- ((6-(pyrrolidin-1-yl)pyridin-2- yl)methyl)-1,9- diazaspiro[5.5]undecan-2-one | L | 2.73 (B) | 421.2 |
| 99 | 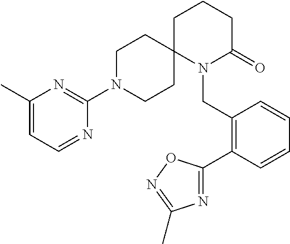 [SMILES: Cc1ccnc(N2CCC3(CCCC(=O)N3Cc3ccccc3-c3nc(C)no3)CC2)n1] | 1-(2-(3-methyl-1,2,4-oxadiazol- 5-yl)benzyl)-9-(4- methylpyrimidin-2-yl)-1,9- diazaspiro[5.5]undecan-2-one | L | 2.78 (C) | 433.2 |
| 100 | 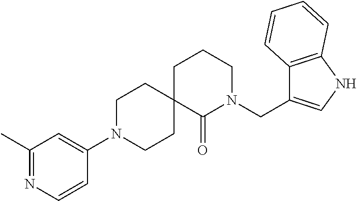 [SMILES: Cc1cc(N2CCC3(CCCN(Cc4c[nH]c5ccccc45)C3=O)CC2)ccn1] | 2-((1H-indol-3-yl)methyl)-9-(2- methylpyridin-4-yl)-2,9- diazaspiro[5.5]undecan-1-one | G | 0.73 (A) | 389.3 |
| 101 | 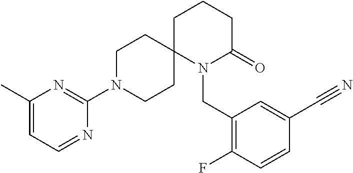 [SMILES: Cc1ccnc(N2CCC3(CCCC(=O)N3Cc3cc(C#N)ccc3F)CC2)n1] | 4-fluoro-3-((9-(4- methylpyrimidin-2-yl)-2-oxo- 1,9-diazaspiro[5.5]undecan-1- yl)methyl)benzonitrile | L | 2.70 (C) | 394.2 |
| 102 | 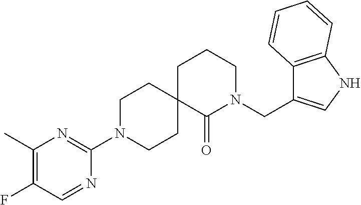 [SMILES: Cc1nc(N2CCC3(CCCN(Cc4c[nH]c5ccccc45)C3=O)CC2)ncc1F] | 2-((1H-indol-3-yl)methyl)-9-(5- fluoro-4-methylpyrimidin-2-yl)- 2,9-diazaspiro[5.5]undecan-1- one | H | 1.19 (A) | 408.4 |
| 103 | 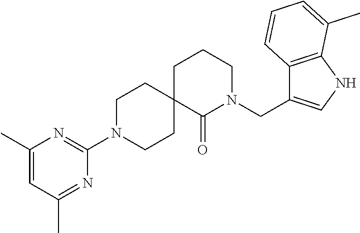 | 9-(4,6-dimethylpyrimidin-2-yl)- 2-((7-methyl-1H-indol-3- yl)methyl)-2,9- diazaspiro[5.5]undecan-1-one | A3 | 1.15 (A) | 418.2 |
| 104 | 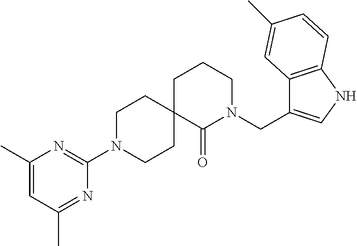 [SMILES: Cc1ccc2[nH]cc(CN3CCCC4(CCN(c5nc(C)cc(C)n5)CC4)C3=O)c2c1] | 9-(4,6-dimethylpyrimidin-2-yl)- 2-((5-methyl-1H-indol-3- yl)methyl)-2,9- diazaspiro[5.5]undecan-1-one | A3 | 1.20 (A) | 418.5 |
| 105 | 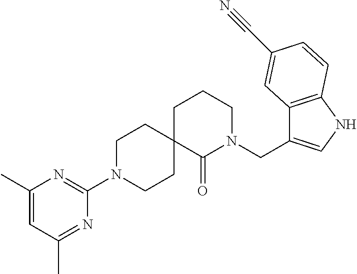 [SMILES: Cc1cc(C)nc(N2CCC3(CCCN(Cc4c[nH]c5ccc(C#N)cc45)C3=O)CC2)n1] | 3-((9-(4,6-dimethylpyrimidin-2- yl)-1-oxo-2,9- diazaspiro[5.5]undecan-2- yl)methyl)-1H-indole-5- carbonitrile | A3 | n.d. | 429.2 (MS) |
| 106 | 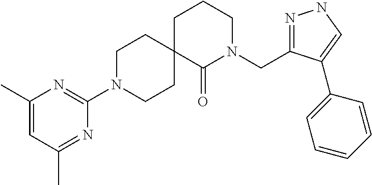 [SMILES: Cc1cc(C)nc(N2CCC3(CCCN(Cc4n[nH]cc4-c4ccccc4)C3=O)CC2)n1] | 9-(4,6-dimethylpyrimidin-2-yl)- 2-((4-phenyl-1H-pyrazol-3- yl)methyl)-2,9- diazaspiro[5.5]undecan-1-one | A | 2.80 (C) | 431.2 |
| 107 | 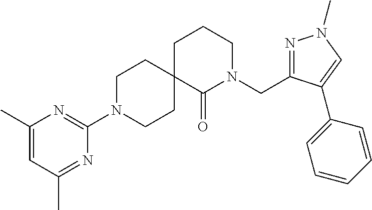 [SMILES: Cc1cc(C)nc(N2CCC3(CCCN(Cc4nn(C)cc4-c4ccccc4)C3=O)CC2)n1] | 9-(4,6-dimethylpyrimidin-2-yl)- 2-((1-methyl-4-phenyl-1H- pyrazol-3-yl)methyl)-2,9- diazaspiro[5.5]undecan-1-one | A1 | 2.95 (C) | 445.2 |
| 108 | 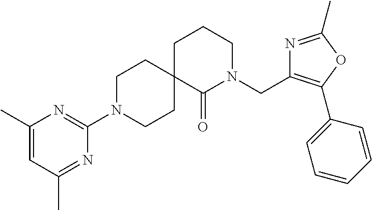 [SMILES: Cc1cc(C)nc(N2CCC3(CCCN(Cc4nc(C)oc4-c4ccccc4)C3=O)CC2)n1] | 9-(4,6-dimethylpyrimidin-2-yl)- 2-((2-methyl-5-phenyloxazol-4- yl)methyl)-2,9- diazaspiro[5.5]undecan-1-one | A | 2.13 (D) | 446.2 |
| 109 | 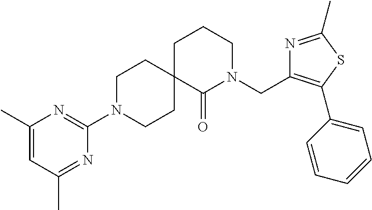 [SMILES: Cc1cc(C)nc(N2CCC3(CCCN(Cc4nc(C)sc4-c4ccccc4)C3=O)CC2)n1] | 9-(4,6-dimethylpyrimidin-2-yl)- 2-((2-methyl-5-phenylthiazol-4- yl)methyl)-2,9- diazaspiro[5.5]undecan-1-one | A | 3.09 (C) | 462.2 |
| 110 | 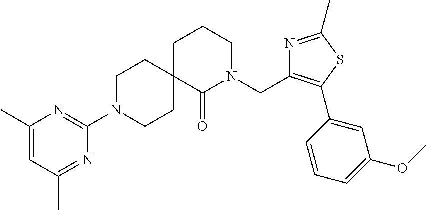 | 9-(4,6-dimethylpyrimidin-2-yl)- 2-((5-(3-methoxyphenyl)-2- methylthiazol-4-yl)methyl)-2,9- diazaspiro[5.5]undecan-1-one | A | 3.13 (C) | 492.2 |
| 111 | 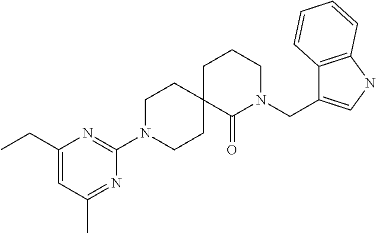 [SMILES: CCc1cc(C)nc(N2CCC3(CCCN(Cc4c[nH]c5ccccc45)C3=O)CC2)n1] | 2-((1H-indol-3-yl)methyl)-9-(4- ethyl-6-methylpyrimidin-2-yl)- 2,9-diazaspiro[5.5]undecan-1- one | H | 1.22 (A) | 418.4 |
| 112 | 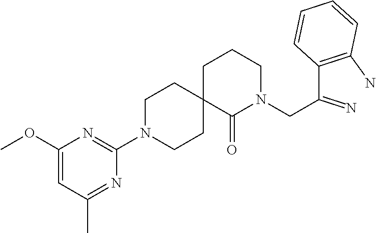 [SMILES: COc1cc(C)nc(N2CCC3(CCCN(CC(=N)c4ccccc4N)C3=O)CC2)n1] | 2-((1H-indazol-3-yl)methyl)-9- (4-methoxy-6-methylpyrimidin- 2-yl)-2,9- diazaspiro[5.5]undecan-1-one | D | 0.88 (A) | 421.4 |
| 113 | 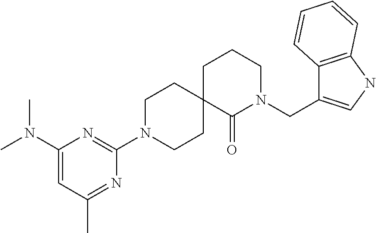 [SMILES: Cc1cc(N(C)C)nc(N2CCC3(CCCN(Cc4c[nH]c5ccccc45)C3=O)CC2)n1] | 2-((1H-indol-3-yl)methyl)-9-(4- (dimethylamino)-6- methylpyrimidin-2-yl)-2,9- diazaspiro[5.5]undecan-1-one | H | 0.86 (A) | 433.4 |
| 114 | 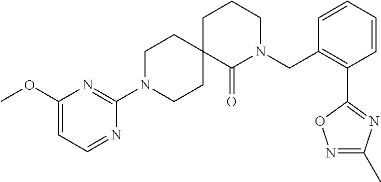 | 9-(4-methoxypyrimidin-2-yl)-2- (2-(3-methyl-1,2,4-oxadiazol-5- yl)benzyl)-2,9- diazaspiro[5.5]undecan-1-one | A | 1.06 (A) | 449.3 |
| 115 | 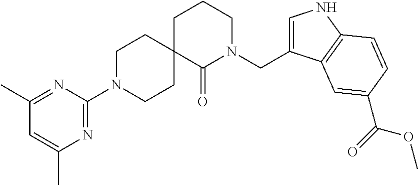 | Methyl 3-((9-(4,6- dimethylpyrimidin-2-yl)-1-oxo- 2,9-diazaspiro[5.5]undecan-2- yl)methyl)-1H-indole-5- carboxylate | A³ | 1.06 (A) | 462.3 |
| 116 | 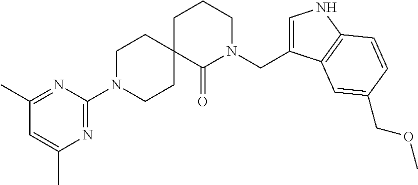 | 9-(4,6-dimethylpyrimidin-2-yl)-2- ((5-(methoxymethyl)-1H-indol-3- yl)methyl)-2,9- diazaspiro[5.5]undecan-1-one | A⁴ | 1.09 (A) | 448.4 |
| 117 | 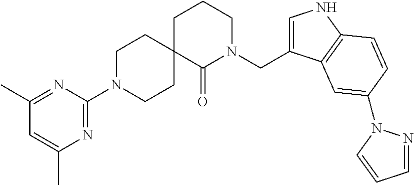 [SMILES: Cc1cc(C)nc(N2CCC3(CCCN(Cc4c[nH]c5ccc(-n6cccn6)cc45)C3=O)CC2)n1] | 2-((5-(1H-pyrazol-1-yl)-1H- indol-3-yl)methyl)-9-(4,6- dimethylpyrimidin-2-yl)-2,9- diazaspiro[5.5]undecan-1-one | A¹ | 1.11 (A) | 470.4 |
| 120 | 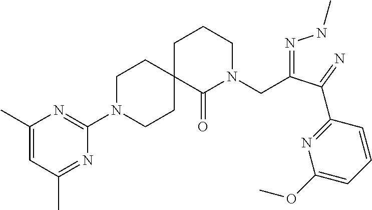 [SMILES: CN/N=C(/CN1CCCC2(CCN(c3nc(C)cc(C)n3)CC2)C1=O)C(=N)c1cccc(OC)n1] | 9-(4,6-dimethylpyrimidin-2-yl)- 2-((5-(6-methoxypyridin-2-yl)- 2-methyl-2H-1,2,3-triazol-4- yl)methyl)-2,9- diazaspiro[5.5]undecan-1-one | N | 1.19 (A) | 477.3 |
| 121 | 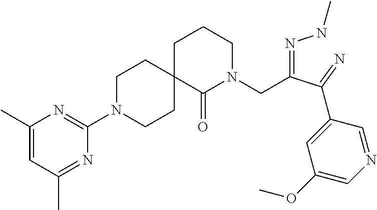 [SMILES: CN/N=C(/CN1CCCC2(CCN(c3nc(C)cc(C)n3)CC2)C1=O)C(=N)c1cncc(OC)c1] | 9-(4,6-dimethylpyrimidin-2-yl)- 2-((5-(5-methoxypyridin-3-yl)- 2-methyl-2H-1,2,3-triazol-4- yl)methyl)-2,9- diazaspiro[5.5]undecan-1-one | N | 0.95 (A) | 477.3 |
| 122 | 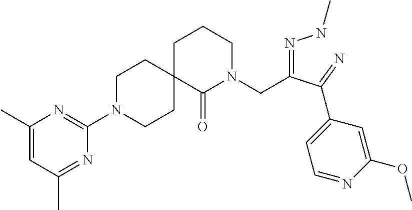 | 9-(4,6-dimethylpyrimidin-2-yl)- 2-((5-(2-methoxypyridin-4-yl)- 2-methyl-2H-1,2,3-triazol-4- yl)methyl)-2,9- diazaspiro[5.5]undecan-1-one | N | 1.09 (A) | 477.3 |
| 123 | 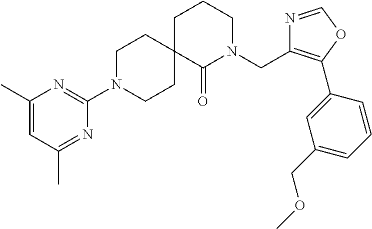 [SMILES: COCc1cccc(-c2ocnc2CN2CCCC3(CCN(c4nc(C)cc(C)n4)CC3)C2=O)c1] | 9-(4,6-dimethylpyrimidin-2-yl)- 2-((5-(3- (methoxymethyl)phenyl)oxazol- 4-yl)methyl)-2,9- diazaspiro[5.5]undecan-1-one | A | 2.98 (A) | 476.2 |
| 124 | 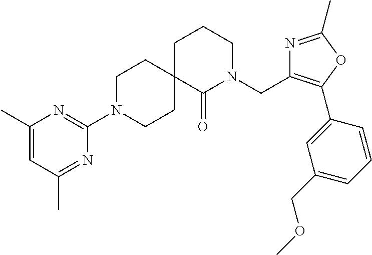 [SMILES: COCc1cccc(-c2oc(C)nc2CN2CCCC3(CCN(c4nc(C)cc(C)n4)CC3)C2=O)c1] | 9-(4,6-dimethylpyrimidin-2-yl)- 2-((5-(3- (methoxymethyl)phenyl)-2- methyloxazol-4-yl)methyl)-2,9- diazaspiro[5.5]undecan-1-one | A | 3.04 (C) | 490.2 |
| 125 | 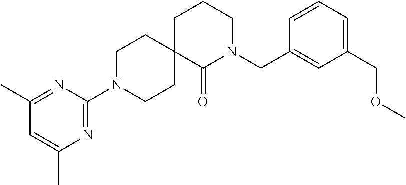 | 9-(4,6-dimethylpyrimidin-2-yl)- 2-(3-(methoxymethyl)benzyl)- 2,9-diazaspiro[5.5]undecan-1- one | A | 2.89 (C) | 409.2 |
| 126 | 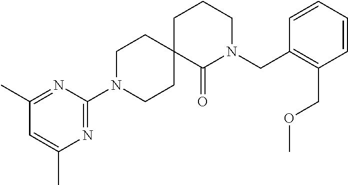 [SMILES: COCc1ccccc1CN1CCCC2(CCN(c3nc(C)cc(C)n3)CC2)C1=O] | 9-(4,6-dimethylpyrimidin-2-yl)- 2-(2-(methoxymethyl)benzyl)- 2,9-diazaspiro[5.5]undecan-1- one | A | 2.91 (C) | 409.2 |
| 127 | 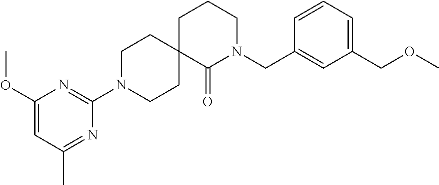 [SMILES: COCc1cccc(CN2CCCC3(CCN(c4nc(C)cc(OC)n4)CC3)C2=O)c1] | 9-(4-methoxy-6- methylpyrimidln-2-yl)-2-(3- (methoxymethyl)benzyl)-2,9- diazaspiro[5.5]undecan-1-one | A | 1.00 (A) | 425.5 |

¹synthesized according to synthesis method D and alkylation with methyl iodide in the last step (see example 6)
²synthesized in analogy to example 6 (using ethyl iodide as alkylation reagent)
³synthesized in analogy to method A using an N-tosyl protected building block, followed by deprotection in analogy to method D
⁴obtained through modification of the ester group in N-tosyl protected example 115 (hydrolysis to the carboxylic acid, formation of the mixed anhydride with ethyl formate, reduction with sodium borohydride to the alcohol, followed by methylation using methyliodide) and deprotection at the nitrogen.

### Building block B2, L16, B23-B26: 2-(2-(bromomethyl)phenyl)-2H-1,2,3-triazole

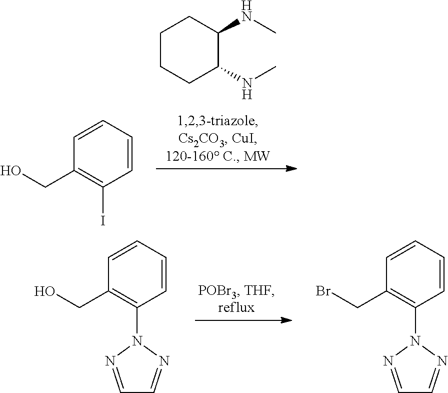

### a) (2-(2H-1,2,3-triazol-2-yl)phenyl)methanol

To a mixture of (2-iodophenyl)methanol (1.50 g, 6.41 mmol), 1H-1,2,3-triazole (0.797 g, 11.54 mmol), trans-N,N′-dimethylcyclohexane-1,2-diamine (0.091 g, 0.641 mmol) and Cs₂CO₃ (3.76 g, 11.54 mmol) in DMF (15 mL), CuI (0.61 g, 3.20 mmol) was added and the reaction mixture was stirred for 20 min at 120° C. and 15 min at 160° C. in the microwave. The reaction mixture was cooled to it and filtered to remove the solids. The filtrate was concentrated under reduced pressure. The residue was purified by flash-column chromatography over silicagel (eluent: gradient 10%-100% ethyl acetate/heptane) to yield the title compound (1.46 g, 64%). [¹H NMR (400 MHz, DMSO-d₆) δ ppm 8.10 (s, 2H), 7.74 (d, J=7.53 Hz, 1H), 7.61 (dd, J=8.03, 1.25 Hz, 1H), 7.52 (td, J=7.53, 1.25 Hz, 1H), 7.44 (m, 1H), 5.26 (t, J=5.40 Hz, 1H), 4.59 (d, J=5.02 Hz, 2H); LCMS Rt_{A}=0.68, [M+H]⁺=176.1].

### b) 2-(2-(bromomethyl)phenyl)-2H-1,2,3-triazole

To a stirred solution of (2-(2H-1,2,3-triazol-2-yl)phenyl)methanol (37 mg, 0.209 mmol) in THF (1 mL), PBr₃ (0.024 mL, 0.251 mmol) was added and the mixture was heated at 70° C. for 20 min. The mixture was cooled to rt, poured into saturated aqueous NaHCO₃ solution and extracted with dichloromethane (2×). The organic layer was dried over anhydrous Na₂SO₄, filtered and concentrated under reduced pressure. The residue was purified by flash-column chromatography over silicagel (eluent: gradient 10%-100% ethyl acetate/heptane) to yield the title compound (28 mg, 54%). [¹H NMR (400 MHz, DMSO-d₆) δ ppm 8.17 (s, 2H), 7.69 (ddd, J=7.59, 3.58, 1.63 Hz, 2H), 7.53 (dqd, J=14.74, 7.51, 7.51, 7.51, 1.63 Hz, 2H), 4.96 (s, 2H); LCMS Rt_{A}=1.02, [M+H]⁺=238.2/240.2].

### Building block F8, A17, A18: 3-(chloromethyl)-1-tosyl-1H-pyrrolo[2,3-b]pyridine

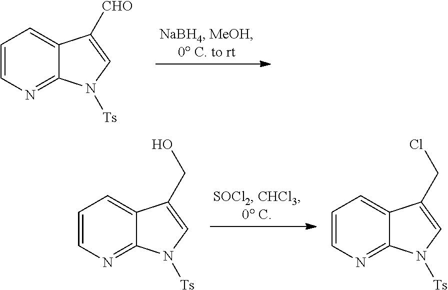 [SMILES: Cc1ccc(S(=O)(=O)n2cc(C=O)c3cccnc32)cc1 ; Cc1ccc(S(=O)(=O)n2cc(CO)c3cccnc32)cc1 ; Cc1ccc(S(=O)(=O)n2cc(CCl)c3cccnc32)cc1]

### a) (1-tosyl-1H-pyrrolo[2,3-b]pyridin-3-yl)methanol

To a stirred solution of 1-tosyl-1H-pyrrolo[2,3-b]pyridine-3-carbaldehyde (2.0 g, 6.66 mmol) in methanol (20 mL) NaBH₄ (0.756 g, 20 mmol) was added in portions at 0° C. Then the mixture was allowed to warm to rt and stirred for 18 h. Methanol was evaporated under reduced pressure and water was added to the stirred residue. The resulting precipitate was filtered off and dried under vacuum to yield the title compound as a white solid (1.6 g, 80%).

[¹H NMR (400 MHz, CHLOROFORM-d) δ ppm 8.45 (dd, J=4.8, 3.2 Hz, 1H), 8.07 (d, J=8.4 Hz, 2H), 7.96 (dd, J=8.0, 1.6 Hz, 1H), 7.70 (s, 1H), 7.26 (d, J=8.4 Hz, 2H), 7.20 (dd, J=7.6, 4.8 Hz, 1H), 4.83 (d, J=5.2 Hz, 2H), 2.37 (s, 3H), 1.73 (t, J=5.2 Hz, 1H); LCMS Rt_{E}=0.85, [M+H]+=302.8].

### b) 3-(chloromethyl)-1-tosyl-1H-pyrrolo[2,3-b]pyridine

To a stirred solution of (1-tosyl-1H-pyrrolo[2,3-b]pyridin-3-yl)methanol (100 mg, 0.331 mmol) in THF (5 mL) POCl₃ (76.2 mg, 0.496 mmol) was added dropwise at 0° C. and stirred for 15 min and then refluxed for 2 h. Then the reaction mixture was quenched with saturated aqueous NaHCO₃ solution and extracted with ethyl acetate (2×). The organic layer was dried over anhydrous Na₂SO₄, filtered and concentrated under reduced pressure at 45° C. The title compound was obtained as a solid (80 mg, 76%). Due to the instability, this compound was used immediately for the next step. [LCMS Rt_{E}=1.75, [M+H]⁺=320.7].

### Building block B27, B29-B34, G73: 2-(3-(bromomethyl)phenyl)pyrimidine

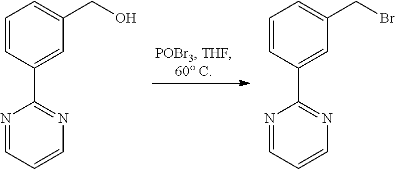 [SMILES: OCc1cccc(-c2ncccn2)c1 ; BrCc1cccc(-c2ncccn2)c1]

To a stirred solution of (3-(pyrimidin-2-yl)phenyl)methanol (2.48 g, 12.92 mmol) in THF (25 mL), POBr₃ (4.81 g, 16.79 mmol) was added and the mixture was heated at 60° C. for 90 min. The mixture was cooled to rt, poured into saturated aqueous NaHCO₃ solution and extracted with ethyl acetate (2×). The combined organic layers were dried over anhydrous Na₂SO₄, filtered and concentrated under reduced pressure. The residue was purified by flash-column chromatography over silicagel (eluent: gradient 5%-40% ethyl acetate/heptane) to yield the title compound (2.59 g, 80%). [¹H NMR (400 MHz, DMSO-d₆) δ ppm 8.92 (d, J=5.0 Hz, 2H), 8.48 (t, J=1.6 Hz, 1H), 8.32 (dt, J=7.7, 1.4 Hz, 1H), 7.58-7.62 (m, 1H), 7.49-7.54 (m, 1 H), 7.47 (t, J=4.8 Hz, 1H), 4.82 (s, 2H); LCMS Rt_{A}=1.04, [M+H]⁺=249.2/251.2].

### Building block B28, B35-B39: 2-(3-(bromomethyl)phenyl)-5-methyloxazole

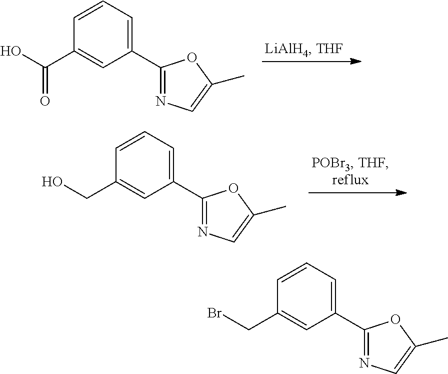

### a) (3-(5-methyloxazol-2-yl)phenyl)methanol

To a stirred suspension of LiAlH₄ (0.29 g, 7.41 mmol) in THF (12 mL) a solution of 3-(5-methyloxazol-2-yl)benzoic acid (1.21 g, 5.84 mmol) in THF (3 mL) was added dropwise at 25° C. (exothermic) and the mixture was stirred at 35° C. for 30 min. The mixture was poured into 1N aqueous HCl solution and extracted with ethyl acetate (2×). The combined organic organic layers were washed with brine, dried over anhydrous Na₂SO₄, filtered and concentrated under reduced pressure. The residue was purified by flash-column chromatography over silicagel (eluent: gradient 10%-100% ethyl acetate/heptane) to yield the title compound (0.88 g, 80%). [¹H NMR (400 MHz, CHLOROFORM-d) δ ppm 8.00 (s, 1 H), 7.87-7.93 (m, 1H), 7.42 (d, J=4.8 Hz, 2H), 6.82 (s, 1H), 4.75 (br. s., 2H), 2.39 (s, 3 H); LCMS R₁₋₄=0.75, [M+H]⁺=190.1].

### b) 2-(3-(bromomethyl)phenyl)-5-methyloxazole

To a stirred solution of (3-(5-methyloxazol-2-yl)phenyl)methanol (0.79 g, 4.13 mmol) in THF (15 mL), POBr₃ (1.93 g, 6.61 mmol) was added in portions at 5° C. The reaction mixture was allowed to warm to rt and then heated at 60° C. for 4 h. The mixture was cooled to rt, poured into saturated aqueous NaHCO₃ solution and extracted with TBME (2×). The combined organic layers were dried over anhydrous Na₂SO₄, filtered and concentrated under reduced pressure. The residue was purified by flash-column chromatography over silicagel (eluent: gradient 5%-40% ethyl acetate/heptane) to yield the title compound (0.88 g, 84%). [¹H NMR (400 MHz, CHLOROFORM-d) δ ppm 8.01-8.07 (m, 1H), 7.93 (ddd, J=6.7, 2.1, 1.9 Hz, 1H), 7.39-7.47 (m, 2H), 6.85 (d, J=1.3 Hz, 1H), 4.53 (s, 2H), 2.41 (d, J=1.3 Hz, 3 H); LCMS Rt_{A}=1.10, [M+H]⁺=252.1/254.1].

### Building block G72: 2-chloro-6-(methoxymethyl)pyridine

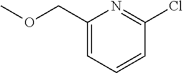

To a solution of 2-chloro-6-(chloromethyl)pyridine (120 mg, 0.70 mmol) in anhydrous MeOH (3 ml) was added sodium methoxide (82 mg, 1.5 mmol) under argon. The reaction mixture was stirred at 50° C. for 24 h. The reaction mixture was quenched with water and extracted with dichloromethane. The organic layer was dried over anhydrous sodium sulfate, filtered and concentrated under reduced pressure to afford the title compound as a colorless oil [¹H NMR (400 MHz, DMSO-d₆) δ ppm 7.85 (t, J=7.8 Hz, 1H), 7.40 (d, J=7.8 Hz, 1H), 7.39 (d, J=7.8 Hz, 1H), 4.44 (s, 2H), 3.34 (s, 3H)].

### Building block L93: 2-(3-(bromomethyl)phenyl)-2H-1,2,3-triazole

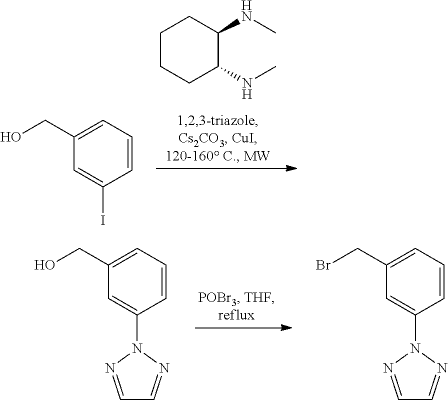

### a) (3-(2H-1,2,3-triazol-2-yl)phenyl)methanol

The title compound was synthesized from (3-iodophenyl)methanol in analogy to (2-(2H-1,2,3-triazol-2-yl)phenyl)methanol (described for Building block B2, L16, B23-B26).

[¹H NMR (400 MHz, DMSO-d₆) δ ppm 8.10 (s, 2H), 7.99-8.03 (m, 1H), 7.84-7.90 (m, 1 H), 7.45-7.54 (m, 1H), 7.30-7.37 (m, 1H), 5.35-5.43 (m, 1H), 4.55-4.63 (m, 2H); LCMS Rt_{A}=0.70, [M+H]⁺=176.2].

### b) 2-(3-(bromomethyl)phenyl)-2H-1,2,3-triazole

The title compound was synthesized from (3-(2H-1,2,3-triazol-2-yl)phenyl)methanol in analogy to 2-(2-(bromomethyl)phenyl)-2H-1,2,3-triazole (described for Building block B2, L16, B23-B26).

[¹H NMR (400 MHz, DMSO-d₆) δ ppm 8.14 (s, 2H), 8.12 (t, J=1.9 Hz, 1H), 7.93-7.98 (m, 1 H), 7.52-7.58 (m, 1H), 7.46-7.52 (m, 1H), 4.82 (s, 2H); LCMS Rt_{A}=1.09, [M+H]⁺ not found].

### Building block L95: 4-(bromomethyl)-2-methyl-1-(phenylsulfonyl)-1H-indole

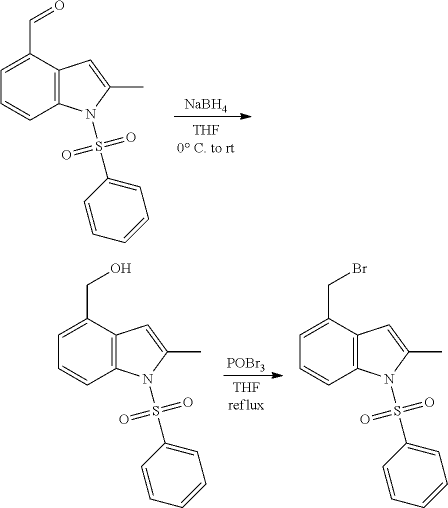

### a) (2-methyl-1-(phenylsulfonyl)-1H-indol-4-yl)methanol

To a solution of 2-methyl-1-(phenylsulfonyl)-1H-indole-4-carbaldehyde (851 mg, 2.84 mmol) in THF (20 ml) and 1 M aqueous NaOH (20 ml) was added under argon at 0° C. NaBH₄ (1.08 g, 28.4 mmol). The reaction mixture was stirred at rt for 15 min. The reaction mixture was extracted with ethyl acetate and the organic phase was washed with water and brine. The combined organic layers were dried over anhydrous sodium sulfate, filtered and evaporated to yield an orange oil. The crude product was purified by flash chromatography (gradient EtOAc/hexane 0:100 to 15:85) to yield 795 mg (93%) of the title compound as a solid. [¹H NMR (600 MHz, DMSO-d₆) δ ppm 7.91 (d, J=8.3 Hz, 1H), 7.85 (d, J=7.5 Hz, 2H), 7.68 (t, J=7.6 Hz, 1H), 7.58 (d, J=7.6 Hz, 1H), 7.56 (d, J=7.6 Hz, 1H), 7.23 (t, J=7.2 Hz, 1H), 7.20 (d, J=7.2 Hz, 1H), 6.68 (s, 1H), 5.19 (t, J=5.4 Hz, 1H), 4.62 (d, J=5.7 Hz, 2H), 2.61 (s, 3 H); LCMS Rt_{D}=2.57 min, [M+Na]⁺=324.0].

### 4-(bromomethyl)-2-methyl-1-(phenylsulfonyl)-1H-indole

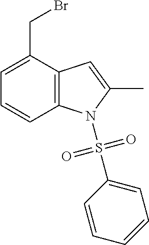 [SMILES: Cc1cc2c(CBr)cccc2n1S(=O)(=O)c1ccccc1]

POBr₃ (342 mg, 1.2 mmol) was added in portions to a solution of (2-methyl-1-(phenylsulfonyl)-1H-indol-4-yl)methanol (300 mg, 0.10 mmol) in dry THF at 0° C. under argon. The reaction mixture was allowed to warm to rt and then was refluxed for 1.25 h. The dark-violet solution was poured into a saturated aqueous sodium bicarbonate solution at 0° C. and the mixture extracted with diethyl ether. The combined organic layers were dried over anhydrous sodium sulfate, filtered and evaporated to yield an orange oil. The crude product was purified by flash chromatography (gradient EtOAc/hexane 0:100 to 20:80) to yield 195 mg (54%) of the title compound as a white powder. [¹H NMR (600 MHz, DMSO-d₆) δ ppm 8.00 (d, J=8.3 Hz, 1H), 7.90 (d, J=7.5 Hz, 2H), 7.71 (t, J=7.5 Hz, 1H), 7.60 (t, J=7.9 Hz, 2 H), 7.32 (d, J=7.3 Hz, 1H), 7.25 (t, J=7.9 Hz, 1H), 6.79 (s, 1H), 4.89 (s, 2H), 2.64 (s, 3H); LCMS Rt_{F}=2.67 min, [M+H]⁺=364.01366.0].

### Building block A123: 4-Bromomethyl-5-(3-methoxymethyl-phenyl)-oxazole

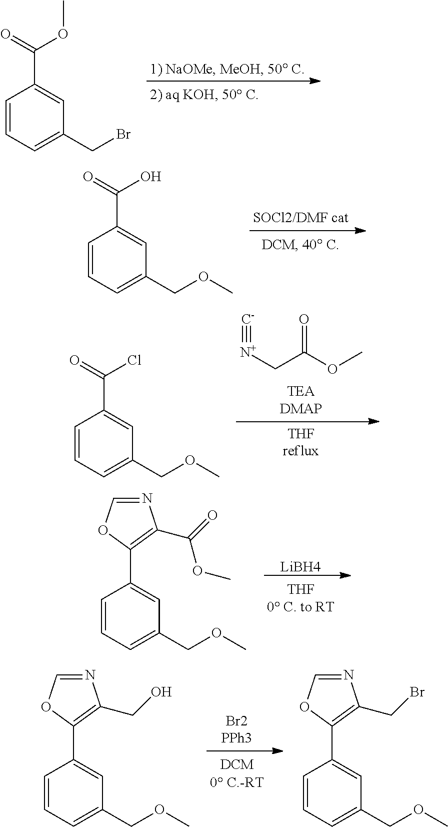

### a) 3-(Methoxymethyl)benzoic acid

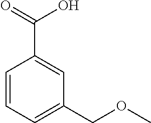

To a solution of methyl 3-(bromomethyl)benzoate (1129-28-8, 1.05 g, 4.6 mmol) in dry MeOH (30 ml) under argon at rt was added sodium methanolate (0.74 g, 13.8 mmol). The suspension was stirred at 50° C. for 2 hr and then aqueous KOH (5M) was added and the mixture again stirred at 50° C. for 1.5 hr.

After cooling to rt aqueous HCl was added to adjust the pH to 2-3. The aqueous phase was extracted with ethyl acetate, the organic phase dried over sodium sulfate, and the solvent was evaporated under reduced pressure to afford the title compound as a colorless oil (749 mg, 96%). LCMS Rt_{B}=2.47 min; [M+H]⁺=167.0]

### b) 3-(Methoxymethyl)benzoyl chloride

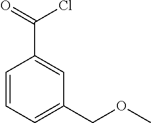

To a solution of 3-(methoxymethyl)benzoic acid (750 mg, 4.4 mmol) in anhydrous DCM (30 ml), DMF (2 drops) and thionyl chloride (0.66 ml, 9.1 mmol) were added. The mixture was stirred at 40° C. under argon for 60 min. More thionyl chloride (0.17 ml, 2.3 mmol) was added and the mixture was stirred for additional 50 min. The solvent was evaporated to give a yellow oil which was used directly in the next step.

### c) Methyl 5-(3-(methoxymethyl)phenyl)oxazole-4-carboxylate

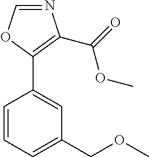 [SMILES: COCc1cccc(-c2ocnc2C(=O)OC)c1]

To a solution of methyl isocyanoacetate (0.50 ml, 5.2 mmol) and 3-(methoxymethyl)benzoyl chloride (4.4 mmol) in anhydrous THF, were added triethylamine (3.5 ml, 25 mmol) and DMAP (0.03 g, 0.23 mmol). The resulting brown mixture was stirred at reflux for 21 h. According to LC-MS 1, the reaction was finished. The mixture was filtered, the residue washed with ethyl acetate and the filtrate was washed with water and brine. The organic phase was dried over sodium sulfate, and the solvent was evaporated under reduced pressure. The crude product was purified by flash column chromatography (gradient: 0-3% methanol in DCM) to yield the title compound as a pale brown oil (0.95 g, 87%). [LCMS Rt_{B}=2.94 min, [M+H]⁺=418.0].

### d) (5-(3-(Methoxymethyl)phenyl)oxazol-4-yl)methanol

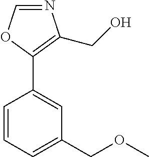 [SMILES: COCc1cccc(-c2ocnc2CO)c1]

To the solution of methyl 5-(3-(methoxymethyl)phenyl)oxazole-4-carboxylate (0.35 g, 1.4 mmol) in dry THF was added lithium borohydride at 0° C. The mixture was allowed to warm to rt and stirred for 2 hr. The reaction was quenched with acetic acid/water (1:1) and diluted with water. The product was extracted with ethyl acetate. The organic phase was washed with water and brine. The combined organic layers were dried over sodium sulfate, filtered and evaporated to give the title compound (0.31 g, 83%) as an orange oil. [LCMS Rt_{B}=2.55 min, [M+H]⁺=220.0].

### e) 4-(Bromomethyl)-5-(3-(methoxymethyl)phenyl)oxazole

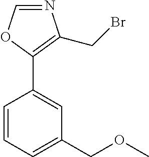 [SMILES: COCc1cccc(-c2ocnc2CBr)c1]

Bromine (0.15 ml, 2.9 mmol) was added dropwise under argon at 0° C. to a solution of triphenylphosphine (0.77 g, 2.9 mmol) in dry DCM (8 ml). After 10 min a solution of (5-(3-(methoxymethyl)phenyl)oxazol-4-yl)methanol in dry DCM (4 ml) was added at 0° C. and the pale yellow solution was allowed to reach rt and stirred for 60 min. The mixture was purified by flash column chromatography (gradient: 0-100% diethyl ether in hexane) to yield the title compound as a pale yellow oil (0.09 g, 27%). [LCMS Rt_{c}=2.52 min, [M+H]⁺=282.01 284.0].

### Building block A124: 4-Bromomethyl-5-(3-methoxymethyl-phenyl)-2-methyl-oxazole

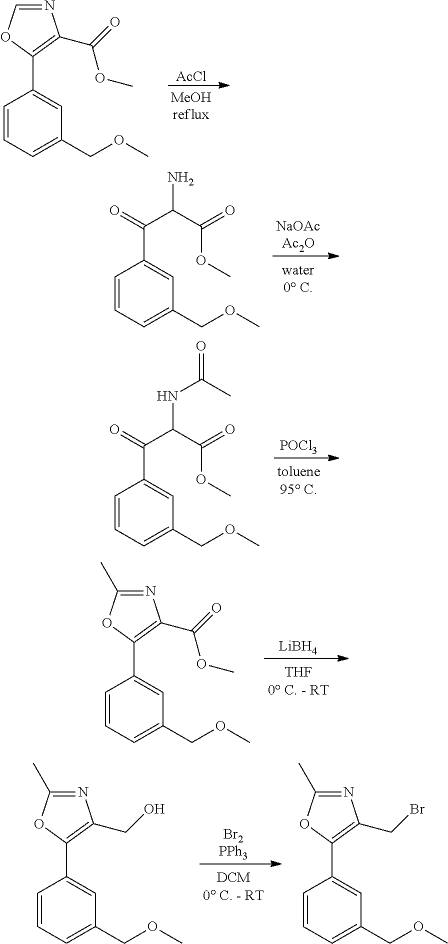

### a) Methyl 2-amino-3-(3-(methoxymethyl)phenyl)-3-oxopropanoate

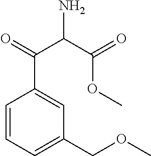 [SMILES: COCc1cccc(C(=O)C(N)C(=O)OC)c1]

To a solution of methyl 5-(3-(methoxymethyl) phenyl)oxazole-4-carboxylate (building block B3.1c, 25 g, 103 mmol) in MeOH was added dropwise acetyl chloride at 0° C. The mixture was stirred at reflux for 24 h. The solvent was evaporated under reduced pressure, the crude product was triturated with acetone, filtered and the residue washed with acetone to give the title compound as a beige solid as its hydrochloride salt (10.6 g, 38%). [LCMS Rt_{A}=2.53 min, [M+H]⁺=238.2].

### b) Methyl 2-acetamido-3-(3-(methoxymethyl)phenyl)-3-oxopropanoate

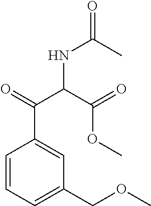 [SMILES: COCc1cccc(C(=O)C(NC(C)=O)C(=O)OC)c1]

To a solution of methyl 2-amino-3-(3-(methoxymethyl)phenyl)-3-oxopropanoate (10.6 g, 39 mmol) and sodium acetate (3.2 g, 39 mmol) in water (310 ml) was added acetic anhydride (7.7 ml, 81 mmol) at 0° C. and the mixture was stirred at 0° C. for 30 min. The mixture was diluted with water and the product was extracted with DCM. The organic phase was washed with water and brine. The organic layer was dried over sodium sulfate, filtered and evaporated to give 10.5 g (97%) of the title compound as brown oil. [LCMS Rt_{B}=2.57 min, [M+H]⁺=280.2].

### c) Methyl 5-(3-(methoxymethyl)phenyl)oxazole-4-carboxylate

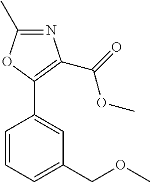

To a solution of methyl 2-acetamido-3-(3-(methoxymethyl)phenyl)-3-oxopropanoate (10.5 g, 37 mmol) in dry toluene (200 ml) was added POCl₃ at room temperature. The solution was stirred at 95° C. for 3 h. After cooling to rt the mixture was poured into water. By addition of an aqueous solution of NaOH (4M) at 0° C. the pH was adjusted to 7. The mixture was extracted with ethyl acetate. The combined organic layers were dried over sodium sulfate, filtered and evaporated to give the title compound as a pale yellow oil (9.2 g, 94%). [LCMS Rt_{B}=3.05 min, [M+H]⁺=262.0].

### d) (5-(3-(Methoxymethyl)phenyl)-2-methyloxazol-4-yl)methanol

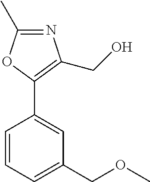

To the solution of methyl 5-(3-(methoxymethyl)phenyl)oxazole-4-carboxylate (9.2 g, 35 mmol) in THF (250 ml) was added at 0° C. LiBH₄ (1.6 g, 70 mmol). The mixture was allowed to reach rt and stirring was continued for 2.5 h. A solution of acetic acid in water (1:1 v/v) was added slowly until the mixture became a clear solution. Stirring was continued for 16 h at rt. The mixture was extracted with DCM, the organic phase was washed with water and brine and the combined organic layers were dried over sodium sulfate, filtered and evaporated to give 6.7 g of a yellow oil which was not further purified.

### e) 4-(Bromomethyl)-5-(3-(methoxymethyl)phenyl)-2-methyloxazole

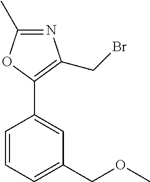

Bromine (3.5 ml, 67 mmol) was added dropwise at 0° C. under argon to a solution of triphenylphosphine (17.7 g, 67 mmol) in dry DCM (250 ml). The pale yellow suspension was stirred at 0° C. for 10 min. The solution of crude (5-(3-(methoxymethyl)phenyl)-2-methyloxazol-4-yl)methanol (6.7 g) in dry DCM (30 ml) was added at 0° C. and the pale yellow solution was allowed to warm to rt and stirring was continued for 1 hr. For completion the reaction mixture was left at 5° C. for 3 days. The reaction mixture was extracted with DCM and the organic phase was washed with water and brine. The combined organic layers were dried over sodium sulfate, filtered and evaporated to give a pale yellow solid. The crude product was purified by flash column chromatography (hexane/diethyl ether 1:1) to yield the title compound as a pale yellow oil (4.0 g, 50%). [LCMS Rtₑ=2.68 min, [M+H]⁺=296.0/298.0].

### Radioligand Binding Assay

For crude cell membrane preparations, cells (CHO, Chinese hamster ovary or HEK, human embryonic kidney) expressing human orexin 1 or human orexin 2 receptors, were washed with HEPES (10 mM, pH 7.5), scraped off the culture plates with the same buffer, and centrifuged at 4° C. for 5 min at 2500×g. The cell pellet was either stored at −80° C. or used directly. Before the experiments, cell membranes were re-suspended in binding assay buffer (10 mM HEPES, 0.5% (w/v) bovine serum albumin, pH 7.5) by homogenisation with a Polytron homogeniser at 50 Hz for 20 s. Cell membranes were also used as made available by commercial providers.

In initial saturation experiments (to calculate Kd and Bmax), cell homogenates (150 μl) were incubated with 0.1 to 15 nM of the radioligand ([³H]-SB649868, 50 μl), 8 concentrations in triplicates in the presence or absence of almorexant (10 μM, 50 μl) to define non specific binding. Bound radioactivity was measured, and data were analysed with the program XLFIT or Graphpad Prism. Protein concentration was determined according to the Bradford/BioRad Protein Assay Kit.

In competition experiments, cell homogenates (150 μl) were incubated in assay buffer (10 mM HEPES, pH 7.5, 0.5% (w/v) bovine serum albumin, 5 mM MgCl₂, 1 mM CaCl₂, and tween 0.05%) for 1 h at room temperature with about 1 nM of the radioligand [³H]-SB649868, 66 Ci/mmole, 50 μl), and with various concentrations of compounds of the invention (50 μl) in triplicates; non-specific binding was determined in the presence of almorexant (10 μM). Reactions were terminated by vacuum filtration, 3 washes of ice cold wash buffer (Tris-HCl pH 7.4/10 mM, with NaCl 154 mM). Competition data is expressed in Table 2 as Kd [μM].

### Calcium Accumulation in Cells (FLIPR):

Cells expressing human orexin 1 or human orexin 2 receptors, were seeded at 8,000 cells/well in 384 well black-walled clear bottom, poly-D-lysine coated plates. After 24 h, the medium was removed and cells were washed once with phosphate buffered saline and serum-deprived overnight in assay buffer (130 mM NaCl, 5.4 mM KCl, 1.8 mM CaCl₂, 0.8 mM MgSO₄, 0.9 mM NaH₂PO₄, 25 mM glucose, 20 mM HEPES, pH 7.4) containing bovine serum albumin (1% w/v).

On the day of the experiment, the cells seeded in black plates were treated with assay buffer containing the Ca²⁺ sensitive fluorescent dye Fluo4-AM (2 μM), and probenecid (0.1 mM). After 1 h plates were washed twice with, and resuspended in, assay buffer containing probenecid (0.1 mM) using a multi plate washer. The plates were placed into a FLIPR II (Fluorometric Imaging Plate Reader, Molecular Devices, Sunnyvale, Calif., USA) and baseline fluorescence (fluorescence light units, FLU) was measured (5 measurements, 2 S each; laser excitation 488 nm at 0.6-1 W, CCD camera exposure 0.4 s) before addition of buffer alone (basal) or containing test compounds (either compound of formula I alone, agonist alone or agonist in the presence of various concentrations of compounds of formula I). Fluorescence measurements were then continued every 1 S for 120 S followed by every 4 S for 240 S.

The measurements were typically made in two sequences:

In the first round, compounds of formula I were tested alone, to confirm that they do not display any significant agonist activity. Compounds of formula I were tested usually in a concentration range from 10⁻⁹ M to 10⁻⁵M.

In the second round, performed one hour later (to allow for equilibration), Orexin A was tested either in the absence (calibration curves, Orexin A agonist controls) or in the presence of compounds of formula I to determine antagonism.

Inhibition data is expressed in Table 2 as Kᵢ [μM], converted by the Cheng and Prusoff correction (Kᵢ=IC₅₀/1+(L/EC₅₀)), where IC₅₀ is the 50% inhibition value determined in concentration response inhibition curves, EC₅₀ is the half maximal activation concentration determined for orexin A in concentration response curves and L is the concentration of orexin A used in inhibition experiments performed in with a submaximal concentration of orexin A in the presence of up to 8 increasing concentrations of compound of formula I. Inhibition data is also expressed in Table 2 as % inhibition value measured at a concentration of 10 μM of selected compounds of formula I.

TABLE 2

| | FLIPR | FLIPR | binding | binding |
|---|---|---|---|---|
| | hOx1R | hOx2R | hOx1R | hOx2R |
| Example | Ki [μM] | Ki [μM] | Kd [μM] | Kd [μM] |
| 1 | 1.621 | 0.007 | 2.022 | 0.066 |
| 2 | 22^{a} | 0.086 | >10 | 0.259 |
| 3 | 0.381 | 0.0009 | 0.483 | 0.012 |
| 4 | 0.135 | 0.0022 | 0.863 | 0.043 |
| 5 | 0.870 | 0.010 | 0.966 | 0.035 |
| 6 | 1.526 | 0.026 | 4.544 | 0.153 |
| 7 | 14^{a} | 0.086 | 7.440 | 0.698 |
| 8 | 31^{a} | 0.276 | n.d. | n.d. |
| 9 | 1.563 | 0.054 | 2.434 | 0.265 |
| 10 | 1.186 | 0.020 | 1.720 | 0.116 |
| 11 | 1.386 | 0.023 | 6.591 | 0.244 |
| 12 | 1.501 | 0.030 | 1.172 | 0.056 |
| 13 | 0.527 | 0.014 | 0.924 | 0.074 |
| 14 | 13^{a} | 0.200 | n.d. | n.d. |
| 15 | 0.855 | 0.013 | 2.829 | 0.109 |
| 16 | 31^{a} | 0.270 | n.d. | n.d. |
| 17 | 12^{a} | 14^{a} | n.d. | n.d. |
| 18 | 3.200 | 0.123 | n.d. | n.d. |
| 19 | 31^{a} | 0.147 | n.d. | n.d. |
| 20 | 2.533 | 0.042 | 8.545 | 0.211 |
| 21 | 26^{a} | 0.247 | n.d. | n.d. |
| 22 | 0.755 | 0.031 | 1.304 | 0.151 |
| 23 | 2.876 | 0.115 | n.d. | n.d. |
| 24 | <10^{a} | 0.509 | n.d. | n.d. |
| 25 | 3.698 | 0.068 | >10 | 0.389 |
| 26 | <10^{a} | 37^{a} | n.d. | n.d. |
| 27 | <10^{a} | 0.645 | n.d. | n.d. |
| 28 | 1.109 | 0.049 | 5.145 | 0.150 |
| 29 | 0.351 | 0.028 | 0.907 | 0.100 |
| 30 | 48^{a} | 0.686 | n.d. | n.d. |
| 31 | 1.612 | 0.092 | 3.806 | 0.145 |
| 32 | 1.857 | 0.059 | >10 | 0.383 |
| 33 | 0.477 | 0.037 | 0.744 | 0.220 |
| 34 | 0.554 | 0.073 | 2.291 | 0.674 |
| 35 | 0.868 | 0.045 | 0.394 | 0.098 |
| 36 | 0.714 | 0.038 | 0.431 | 0.089 |
| 37 | 1.888 | 0.079 | >10 | 1.158 |
| 38 | 0.924 | 0.043 | 1.194 | 0.177 |
| 39 | 0.533 | 0.029 | 1.443 | 0.367 |
| 40 | 0.241 | 0.019 | 0.332 | 0.037 |
| 41 | 1.643 | 0.015 | 2.823 | 0.085 |
| 42 | 34^{a} | 0.026 | 5.235 | 0.140 |
| 43 | 0.729 | 0.017 | 1.860 | 0.612 |
| 44 | 1.568 | 0.012 | 5.737 | 0.238 |
| 45 | 0.521 | 0.010 | 1.057 | 0.064 |
| 46 | 25^{a} | 0.143 | n.d. | n.d. |
| 47 | 25^{a} | 0.630 | n.d. | n.d. |
| 48 | 3.152 | 0.152 | n.d. | n.d. |
| 49 | 2.182 | 0.190 | n.d. | n.d. |
| 50 | 1.861 | 0.223 | n.d. | n.d. |
| 51 | 44^{a} | 0.084 | 4.816 | 0.700 |
| 52 | 1.056 | 0.125 | n.d. | n.d. |
| 53 | 2.745 | 0.420 | n.d. | n.d. |
| 54 | 3.146 | 0.149 | n.d. | n.d. |
| 55 | 40^{a} | 0.727 | n.d. | n.d. |
| 56 | 35^{a} | 0.718 | n.d. | n.d. |
| 57 | 3.745 | 0.171 | n.d. | n.d. |
| 58 | 31^{a} | 0.156 | n.d. | n.d. |
| 59 | 15^{a} | 1.379 | n.d. | n.d. |
| 60 | 3.111 | 0.228 | n.d. | n.d. |
| 61 | 3.837 | 0.390 | n.d. | n.d. |
| 62 | 31^{a} | 0.117 | n.d. | n.d. |
| 63 | 16^{a} | 1.279 | n.d. | n.d. |
| 64 | 3.370 | 0.150 | n.d. | n.d. |
| 65 | 0.699 | 0.066 | >10 | 0.588 |
| 66 | 36^{a} | 0.991 | n.d. | n.d. |
| 67 | <10^{a} | 0.134 | 3.589 | 0.315 |
| 68 | 14^{a} | 0.798 | n.d. | n.d. |
| 69 | 31^{a} | 0.320 | n.d. | n.d. |
| 70 | 1.811 | 0.240 | n.d. | n.d. |
| 71 | 31^{a} | 0.166 | n.d. | n.d. |
| 72 | 16^{a} | 0.280 | n.d. | n.d. |
| 73 | 30^{a} | 0.639 | n.d. | n.d. |
| 74 | 2.147 | 0.160 | >10 | 0.338 |
| 75 | 1.480 | 0.0072 | >10 | 0.466 |
| 76 | <10^{a} | 1.133 | n.d. | n.d. |
| 77 | 3.631 | 0.173 | n.d. | n.d. |
| 78 | 0.481 | 0.003 | 3.419 | 0.216 |
| 79 | 0.708 | 0.126 | n.d. | n.d. |
| 80 | 41^{a} | 0.050 | 7.755 | 1.096 |
| 81 | 2.033 | 0.045 | 0.798 | 0.029 |
| 82 | 0.856 | 0.057 | >10 | 3.163 |
| 83 | 1.518 | 0.013 | >10 | 2.115 |
| 84 | 24^{a} | 49^{a} | n.d. | n.d. |
| 85 | 42^{a} | 0.664 | n.d. | n.d. |
| 86 | 22^{a} | 0.545 | n.d. | n.d. |
| 87 | 20^{a} | 0.351 | n.d. | n.d. |
| 88 | 0.406 | 0.046 | 5.056 | 0.611 |
| 89 | 18^{a} | 0.426 | n.d. | n.d. |
| 90 | <10^{a} | <10^{a} | n.d. | n.d. |
| 91 | 26a | 0.017 | >10 | 0.270 |
| 92 | 2.584 | 0.104 | >10 | 1.146 |
| 93 | 1.400 | 0.016 | 4.134 | 0.091 |
| 94 | 36^{a} | 23^{a} | n.d. | n.d. |
| 95 | 10^{a} | 15^{a} | n.d. | n.d. |
| 96 | 0.866 | 0.018 | 1.942 | 0.090 |
| 97 | 15^{a} | 32^{a} | n.d. | n.d. |
| 98 | <10^{a} | 20^{a} | n.d. | n.d. |
| 99 | 12a | 0.614 | n.d. | n.d. |
| 100 | <10^{a} | <10^{a} | n.d. | n.d. |
| 101 | <10^{a} | <10^{a} | n.d. | n.d. |
| 102 | 1.069 | 0.040 | 1.159 | 0.059 |
| 103 | 1.904 | 0.026 | 1.772 | 0.035 |
| 104 | 0.292 | 0.0048 | 1.023 | 0.025 |
| 105 | 0.295 | 0.0033 | 0.655 | 0.025 |
| 106 | 0.763 | 0.0033 | 1.831 | 0.049 |
| 107 | 0.833 | 0.020 | 2.251 | 0.166 |
| 108 | 0.507 | 0.018 | 0.852 | 0.072 |
| 109 | 1.370 | 0.042 | 2.559 | 0.188 |
| 110 | 0.213 | 0.0038 | 0.236 | 0.007 |
| 111 | 0.761 | 0.011 | 0.835 | 0.044 |
| 112 | 0.255 | 0.0061 | 0.912 | 0.082 |
| 113 | 1.432 | 0.021 | 0.389 | 0.028 |
| 114 | 1.532 | 0.030 | 2.140 | 0.231 |
| 115 | 0.021 | 0.0007 | 0.052 | 0.003 |
| 116 | 0.033 | 0.0003 | 0.094 | 0.002 |
| 117 | 0.037 | 0.0013 | 1.019 | 0.027 |
| 118 | 3.580 | 0.079 | 2.107 | 0.178 |
| 119 | 0.868 | 0.0039 | 1.331 | 0.028 |
| 120 | 29^{a} | 0.032 | >10 | 0.197 |
| 121 | 0.315 | 0.002 | 0.196 | 0.015 |
| 122 | 0.176 | 0.0014 | 0.130 | 0.014 |
| 123 | 1.776 | 0.018 | 3.245 | 0.071 |
| 124 | 0.101 | 0.0012 | 0.368 | 0.019 |
| 125 | 22^{a} | 0.177 | n.d. | n.d. |
| 126 | 31^{a} | 0.140 | n.d. | n.d. |
| 127 | 2.114 | 0.100 | n.d. | n.d. |

n.d. = not determined
^{a}% inhibition value measured at a concentration of 10 μM of compound of formula I.

### The Following are Further Embodiments of the Invention:

### EMBODIMENT 1

A compound of the formula I

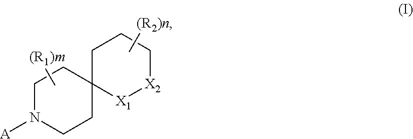

wherein
A is a five- to six-membered aromatic ring system which may contain from 1 to 4 hetero atoms selected from nitrogen, oxygen and sulfur, wherein the ring system is substituted once or more than once by R₃, and wherein a substituent on a nitrogen in a heterocyclic ring system may not be halogen;
each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—;
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m is 0, 1, 2, 3, 4, 5 or 6;
n is 0, 1, 2, 3, 4, 5 or 6;
each R₁ or R₂ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

### —X₁— is —C(O)— and —X₂— is —N(L-B)—;

### or —X₁— is —N(L-B)— and —X₂— is —C(O)—;

### L is —C(R₆)₂—;

each R₆ independently is hydrogen, C₁₋₈alkyl, C₁₋₈halogenalkyl, C₃₋₇cycloalkyl or C₃₋₇cycloalkyl(C₁₋₄alkyl);
or two R₆ together with the carbon atom to which they are bound form a C₃₋₄cycloalkyl;
B is a five- to six-membered monocyclic or eight- to ten-membered fused biycyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;
each R₇ independently is C₁₋₆alkyl, C₁₋₆halogenalkyl, C₁₋₆alkoxy, C₁₋₄alkoxyC₁₋₆alkyl, C₁₋₄alkoxycarbonyl, C₁₋₆halogenalkoxy, halogen, cyano or a three- to seven-membered monocyclic ring system which may be aromatic, saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₄alkoxyC₁₋₆alkyl, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo; in free form or in salt form or in pharmaceutically acceptable salt form.

### EMBODIMENT 2

A compound of formula I according to embodiment 1 in free form or in salt form or in pharmaceutically acceptable salt form, wherein —X₁— is —C(O)— and —X₂— is —N(L-B)—.

### EMBODIMENT 3

A compound of formula I according to embodiment 1 or 2 in free form or in salt form or in pharmaceutically acceptable salt form, wherein m and n are both 0; and each R₆ is hydrogen.

### EMBODIMENT 4

A compound of formula I according to any one of embodiments 1 to 3 in free form or in salt form or in pharmaceutically acceptable salt form, wherein

A is pyrid-2-yl being substituted in the 4-position and/or in the 6-position by R₃;
or A is pyrimidin-2-yl being substituted in the 4-position or in the 4-position and in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄alkoxy-C₁₋₄alkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

### EMBODIMENT 5

A compound of formula I according to any one of embodiments 1 to 4 in free form or in salt form or in pharmaceutically acceptable salt form, wherein B is indol-3-yl which may be substituted once or more than once by R₇, wherein a substituent on the nitrogen of the indol-3-yl may not be halogen; and each R₇ independently is C₁₋₆alkyl, C₁₋₆alkoxy, C₁₋₄alkoxyC₁₋₆alkyl, C₁₋₄alkoxycarbonyl, C₁₋₆halogenalkyl or halogen.

### EMBODIMENT 6

A compound of formula I according to any one of embodiments 1 to 4 in free form or in salt form or in pharmaceutically acceptable salt form, wherein B is a six-membered monocyclic aromatic ring system which may contain 1 to 2 nitrogen atoms, wherein the ring system is substituted once by R₇ₐ, and wherein the ring system may be further substituted once or more than once by R_{7b};

R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆alkyl, C₁₋₆halogenalkyl, C₁₋₆alkoxy, C₁₋₆halogenalkoxy, halogen or cyano.

### EMBODIMENT 7

A compound of formula I according to embodiment 1 in free form or in salt form or in pharmaceutically acceptable salt form, wherein said compound is selected from the group consisting of
9-(4,6-dimethylpyrimidin-2-yl)-2-(2-(3-methyl-1,2,4-oxadiazol-5-yl)benzyl)-2,9-diazaspiro[5.5]undecan-1-one;
2-(2-(2H-1,2,3-triazol-2-yl)benzyl)-9-(4,6-dimethylpyrimidin-2-yl)-2,9-diazaspiro[5.5]undecan-1-one;
1-((1H-indol-4-yl)methyl)-9-(4,6-dimethylpyrimidin-2-yl)-1,9-diazaspiro[5.5]undecan-2-one;
2-((1H-indol-3-yl)methyl)-9-(4-methoxy-6-methylpyrimidin-2-yl)-2,9-diazaspiro[5.5]undecan-1-one;
2-((1H-indol-3-yl)methyl)-9-(4,6-dimethylpyrimidin-2-yl)-2,9-diazaspiro[5.5]undecan-1-one;
9-(4,6-dimethylpyrimidin-2-yl)-2-((1-methyl-1H-indol-3-yl)methyl)-2,9-diazaspiro[5.5]undecan-1-one;
1-((1H-indol-3-yl)methyl)-9-(6-methylpyrazin-2-yl)-1,9-diazaspiro[5.5]undecan-2-one;
2-((1H-pyrrolo[2,3-b]pyridin-3-yl)methyl)-9-(6-methoxypyrazin-2-yl)-2,9-diazaspiro[5.5]undecan-1-one;
2-((1H-indol-3-yl)methyl)-9-(6-methylpyridin-2-yl)-2,9-diazaspiro[5.5]undecan-1-one;
2-(2-((1H-indol-3-yl)methyl)-1-oxo-2,9-diazaspiro[5.5]undecan-9-yl)isonicotinonitrile;
2-((1H-indol-3-yl)methyl)-9-(6-methylpyrazin-2-yl)-2,9-diazaspiro[5.5]undecan-1-one;
2-((1H-indol-3-yl)methyl)-9-(4-methylpyrimidin-2-yl)-2,9-diazaspiro[5.5]undecan-1-one;
2-((1H-indol-3-yl)methyl)-9-(4-methoxypyrimidin-2-yl)-2,9-diazaspiro[5.5]undecan-1-one;
6-(1-((1H-indol-4-yl)methyl)-2-oxo-1,9-diazaspiro[5.5]undecan-9-yl)nicotinonitrile;
9-(4,6-dimethylpyrimidin-2-yl)-2-(2-(furan-2-yl)benzyl)-2,9-diazaspiro[5.5]undecan-1-one;
1-(2-(2H-1,2,3-triazol-2-yl)benzyl)-9-(4-methylpyrimidin-2-yl)-1,9-diazaspiro[5.5]undecan-2-one;
2-((1H-indol-3-yl)methyl)-9-(5-methyl-1,3,4-oxadiazol-2-yl)-2,9-diazaspiro[5.5]undecan-1-one;
2-((1H-pyrrolo[2,3-b]pyridin-3-yl)methyl)-9-(6-chloropyrazin-2-yl)-2,9-diazaspiro[5.5]undecan-1-one;
2-((1H-pyrrolo[2,3-b]pyridin-3-yl)methyl)-9-(6-methylpyrazin-2-yl)-2,9-diazaspiro[5.5]undecan-1-one;
2-((2,3-dihydrobenzo[b][1,4]dioxin-5-yl)methyl)-9-(4,6-dimethylpyrimidin-2-yl)-2,9-diazaspiro[5.5]undecan-1-one;
9-(6-chloropyrazin-2-yl)-2-((2,3-dihydrobenzo[b][1,4]dioxin-5-yl)methyl)-2,9-diazaspiro[5.5]undecan-1-one;
9-(4,6-dimethylpyrimidin-2-yl)-2-(3-(5-methyl-1,2,4-oxadiazol-3-yl)benzyl)-2,9-diazaspiro[5.5]undecan-1-one;
9-(4,6-dimethylpyrimidin-2-yl)-2-((5-methyl-3-phenylisoxazol-4-yl)methyl)-2,9-diazaspiro[5.5]undecan-1-one;
2-(2-(2H-1,2,3-triazol-2-yl)benzyl)-9-(4-methylpyrimidin-2-yl)-2,9-diazaspiro[5.5]undecan-1-one;
2-(2-(2-(2H-1,2,3-triazol-211)benzyl)-1-oxo-2,9-diazaspiro[5.5]undecan-9-yl)-6-methylpyrimidine-4-carbonitrile;
6-(2-(2-(2H-1,2,3-triazol-2-yl)benzyl)-1-oxo-2,9-diazaspiro[5.5]undecan-9-yl)picolinonitrile;
2-(2-(2-(2H-1,2,3-triazol-2-yl)benzyl)-1-oxo-2,9-diazaspiro[5.5]undecan-9-yl)isonicotinonitrile;
2-methyl-6-(1-oxo-2-(3-(pyrimidin-2-yl)benzyl)-2,9-diazaspiro[5.5]undecan-9-yl)isonicotinonitrile;
2-methyl-6-(2-(3-(5-methyloxazol-2-yl)benzyl)-1-oxo-2,9-diazaspiro[5.5]undecan-9-yl)isonicotinonitrile;
9-(4-methylpyrimidin-2-yl)-2-(3-(pyrimidin-2-yl)benzyl)-2,9-diazaspiro[5.5]undecan-1-one;
9-(4,6-dimethylpyrimidin-2-yl)-2-(3-(pyrimidin-2-yl)benzyl)-2,9-diazaspiro[5.5]undecan-1-one;
6-methyl-2-(1-oxo-2-(3-(pyrimidin-2-yl)benzyl)-2,9-diazaspiro[5.5]undecan-9-yl)pyrimidine-4-carbonitrile;
2-(1-oxo-2-(3-(pyrimidin-2-yl)benzyl)-2,9-diazaspiro[5.5]undecan-9-yl)pyrimidine-4-carbonitrile;
6-(1-oxo-2-(3-(pyrimidin-2-yl)benzyl)-2,9-diazaspiro[5.5]undecan-9-yl)picolinonitrile;
2-(1-oxo-2-(3-(pyrimidin-2-yl)benzyl)-2,9-diazaspiro[5.5]undecan-9-yl)isonicotinonitrile;
9-(4,6-dimethylpyrimidin-2-yl)-2-(3-(5-methyloxazol-2-yl)benzyl)-2,9-diazaspiro[5.5]undecan-1-one;
6-methyl-2-(2-(3-(5-methyloxazol-2-yl)benzyl)-1-oxo-2,9-diazaspiro[5.5]undecan-9-yl)pyrimidine-4-carbonitrile;
2-(3-(5-methyloxazol-2-yl)benzyl)-9-(4-methylpyrimidin-2-yl)-2,9-diazaspiro[5.5]undecan-1-one;
6-(2-(3-(5-methyloxazol-2-yl)benzyl)-1-oxo-2,9-diazaspiro[5.5]undecan-9-yl)picolinonitrile;
2-(2-(3-(5-methyloxazol-2-yl)benzyl)-1-oxo-2,9-diazaspiro[5.5]undecan-9-yl)isonicotinonitrile;
1-((1H-indol-4-yl)methyl)-9-(6-methylpyrazin-2-yl)-1,9-diazaspiro[5.5]undecan-2-one;
1-((1H-indol-4-yl)methyl)-9-(4-methylpyrimidin-2-yl)-1,9-diazaspiro[5.5]undecan-2-one;
1-((1H-indol-4-yl)methyl)-9-(6-methoxypyrazin-2-yl)-1,9-diazaspiro[5.5]undecan-2-one;
1-((1H-indol-4-yl)methyl)-9-(6-chloropyrazin-2-yl)-1,9-diazaspiro[5.5]undecan-2-one;
2-((1H-indazol-3-yl)methyl)-9-(4,6-dimethylpyrimidin-2-yl)-2,9-diazaspiro[5.5]undecan-1-one;
2-((1H-indol-3-yl)methyl)-9-(4-(dimethylamino)pyrimidin-2-yl)-2,9-diazaspiro[5.5]undecan-1-one;
2-((1H-indol-3-yl)methyl)-9-(2-methoxypyrimidin-4-yl)-2,9-diazaspiro[5.5]undecan-1-one;
2-((1H-indol-3-yl)methyl)-9-(2-chloropyrimidin-4-yl)-2,9-diazaspiro[5.5]undecan-1-one;
2-((1H-indol-3-yl)methyl)-9-(4-(trifluoromethyl)pyrimidin-2-yl)-2,9-diazaspiro[5.5]undecan-1-one;
2-((1H-indol-3-yl)methyl)-9-(4-isopropylpyrimidin-2-yl)-2,9-diazaspiro[5.5]undecan-1-one;
2-((1H-indol-3-yl)methyl)-9-(4-chloropyrimidin-2-yl)-2,9-diazaspiro[5.5]undecan-1-one;
2-((1H-indol-3-yl)methyl)-9-(4-ethoxypyrimidin-2-yl)-2,9-diazaspiro[5.5]undecan-1-one;
9-(6-chloropyrazin-2-yl)-2-((1-methyl-1H-indol-3-yl)methyl)-2,9-diazaspiro[5.5]undecan-1-one;
9-(4,6-dimethylpyrimidin-2-yl)-2-((1-ethyl-1H-indol-3-yl)methyl)-2,9-diazaspiro[5.5]undecan-1-one;
1-((1H-indol-3-yl)methyl)-9-(6-nnethoxypyrazin-2-yl)-1,9-diazaspiro[5.5]undecan-2-one;
2-((2,3-dihydrobenzo[b][1,4]dioxin-5-yl)methyl)-9-(6-methoxypyrazin-2-yl)-2,9-diazaspiro[5.5]undecan-1-one;
2-((1H-indol-3-yl)methyl)-9-m-tolyl-2,9-diazaspiro[5.5]undecan-1-one;
2-((1H-indol-3-yl)methyl)-9-(5-methylpyridin-3-yl)-2,9-diazaspiro[5.5]undecan-1-one;
2-((1H-indol-3-yl)methyl)-9-(3,4-dimethoxyphenyl)-2,9-diazaspiro[5.5]undecan-1-one;
2-((1H-indol-3-yl)methyl)-9-(3-methoxyphenyl)-2,9-diazaspiro[5.5]undecan-1-one;
2-((1H-indol-3-yl)methyl)-9-(2-methoxypyridin-4-yl)-2,9-diazaspiro[5.5]undecan-1-one;
2-((1H-indol-3-yl)methyl)-9-(5-methoxypyridin-3-yl)-2,9-diazaspiro[5.5]undecan-1-one;
2-((1H-indol-3-yl)methyl)-9-(2,6-dimethylpyridin-4-yl)-2,9-diazaspiro[5.5]undecan-1-one;
2-((1H-indol-3-yl)methyl)-9-(5,6-dimethylpyridin-2-yl)-2,9-diazaspiro[5.5]undecan-1-one;
2-((1H-indol-3-yl)methyl)-9-(6-methoxypyridin-2-yl)-2,9-diazaspiro[5.5]undecan-1-one;
2-((1H-indol-3-yl)methyl)-9-(3-methylpyridin-2-yl)-2,9-diazaspiro[5.5]undecan-1-one;
2-((1H-indol-3-yl)methyl)-9-(4-methylpyridin-2-yl)-2,9-diazaspiro[5.5]undecan-1-one;
2-((1H-indol-3-yl)methyl)-9-(5-methylpyridin-2-yl)-2,9-diazaspiro[5.5]undecan-1-one;
6-(2-((1H-indol-3-yl)methyl)-1-oxo-2,9-diazaspiro[5.5]undecan-9-yl)nicotinonitrile;
2-((1H-indol-3-yl)methyl)-9-(2-methylpyrimidin-4-yl)-2,9-diazaspiro[5.5]undecan-1-one;
2-((1H-indol-3-yl)methyl)-9-(2,6-dimethylpyrimidin-4-yl)-2,9-diazaspiro[5.5]undecan-1-one;
2-((1H-indol-3-yl)methyl)-9-(5-methylpyrazin-2-yl)-2,9-diazaspiro[5.5]undecan-1-one;
2-((1H-indol-3-yl) methyl)-9-(6-(methoxymethyl)pyridin-2-yl)-2,9-diazaspiro[5.5]undecan-1-one;
9-(4,6-dimethylpyridin-2-yl)-2-(3-(pyrimidin-2-yl)benzyl)-2,9-diazaspiro[5.5]undecan-1-one;
2-(2-((1H-indol-3-yl)methyl)-1-oxo-2,9-diazaspiro[5.5]undecan-9-yl)-6-methylpyrimidine-4-carbonitrile;
2-(2-((1H-indol-3-yl)methyl)-1-oxo-2,9-diazaspiro[5.5]undecan-9-yl)pyrimidine-4,6-dicarbonitrile;
6-(2-((1H-indol-3-yl)methyl)-1-oxo-2,9-diazaspiro[5.5]undecan-9-yl)pyrazine-2-carbonitrile;
2-(2-((1H-indol-3-yl)methyl)-1-oxo-2,9-diazaspiro[5.5]undecan-9-yl)-6-methylisonicotinonitrile;
2-((1H-indol-3-yl)methyl)-9-(4-ethylpyrimidin-2-yl)-2,9-diazaspiro[5.5]undecan-1-one;
2-(2-((1H-indol-3-yl)methyl)-1-oxo-2,9-diazaspiro[5.5]undecan-9-yl)pyrimidine-4-carbonitrile;
2-((1H-indol-3-yl)methyl)-9-(4,5-dimethylpyrimidin-2-yl)-2,9-diazaspiro[5.5]undecan-1-one;
2-((1H-indol-3-yl)methyl)-9-(6-methoxypyrazin-2-yl)-2,9-diazaspiro[5.5]undecan-1-one;
6-(2-((1H-indol-3-yl)methyl)-1-oxo-2,9-diazaspiro[5.5]undecan-9-yl)picolinonitrile;
2-((1H-indol-3-yl)methyl)-9-(6-hydroxypyridin-2-yl)-2,9-diazaspiro[5.5]undecan-1-one;
2-((1H-indol-3-yl)methyl)-9-(5-methyl-1,3,4-thiadiazol-2-yl)-2,9-diazaspiro[5.5]undecan-1-one;
2-((1H-indol-3-yl)methyl)-9-(3,6-dimethylpyrazin-2-yl)-2,9-diazaspiro[5.5]undecan-1-one;
2-((1H-indol-3-yl)methyl)-9-(6-methoxypyrimidin-4-yl)-2,9-diazaspiro[5.5]undecan-1-one;
2-((1H-indol-3-yl)methyl)-9-(4,6-dimethoxypyrimidin-2-yl)-2,9-diazaspiro[5.5]undecan-1-one;
2-((1H-indol-3-yl)methyl)-9-(5-(trifluoromethyl)pyridin-2-yl)-2,9-diazaspiro[5.5]undecan-1-one;
9-(4-methylpyrimidin-2-yl)-1-(4-(pyrimidin-2-yl)benzyl)-1,9-diazaspiro[5.5]undecan-2-one;
2-(1-((1H-indol-4-yl)methyl)-2-oxo-1,9-diazaspiro[5.5]undecan-9-yl)isonicotinonitrile;
6-(1-((1H-indol-4-yl)methyl)-2-oxo-1,9-diazaspiro[5.5]undecan-9-yl)picolinonitrile;
9-(4,6-dimethylpyrimidin-2-yl)-2-(2-(oxazol-2-yl)benzyl)-2,9-diazaspiro[5.5]undecan-1-one;
1-(3-(2H-1,2,3-triazol-2-yl)benzyl)-9-(4-methylpyrimidin-2-yl)-1,9-diazaspiro[5.5]undecan-2-one;
9-(4-methylpyrimidin-2-yl)-1-((5-phenyl-1,3,4-oxadiazol-2-yl)methyl)-1,9-diazaspiro[5.5]undecan-2-one;
1-((2-methyl-1H-indol-4-yl)methyl)-9-(4-methylpyrimidin-2-yl)-1,9-diazaspiro[5.5]undecan-2-one;
1-(3-(3-methyl-1,2,4-oxadiazol-5-yl)benzyl)-9-(4-methylpyrimidin-2-yl)-1,9-diazaspiro[5.5]undecan-2-one;
9-(4-methylpyrimidin-2-yl)-1-((6-(pyrrolidin-1-yl)pyridin-2-yl)methyl)-1,9-diazaspiro[5.5]undecan-2-one;
1-(2-(3-methyl-1,2,4-oxadiazol-5-yl)benzyl)-9-(4-methylpyrimidin-2-yl)-1,9-diazaspiro[5.5]undecan-2-one;
2-((1H-indol-3-yl)methyl)-9-(2-methylpyridin-4-yl)-2,9-diazaspiro[5.5]undecan-1-one;
4-fluoro-3-((9-(4-methylpyrimidin-2-yl)-2-oxo-1,9-diazaspiro[5.5]undecan-1-yl)methyl)benzonitrile;
2-((1H-indol-3-yl)methyl)-9-(5-fluoro-4-methylpyrimidin-2-yl)-2,9-diazaspiro[5.5]undecan-1-one;
9-(4,6-dimethylpyrimidin-2-yl)-2-((7-methyl-1H-indol-3-yl)methyl)-2,9-diazaspiro[5.5]undecan-1-one;
9-(4,6-dimethylpyrimidin-2-yl)-2-((5-methyl-1H-indol-3-yl)methyl)-2,9-diazaspiro[5.5]undecan-1-one;
3-((9-(4,6-dimethylpyrimidin-2-yl)-1-oxo-2,9-diazaspiro[5.5]undecan-2-yl)methyl)-1H-indole-5-carbonitrile;
9-(4,6-dimethylpyrimidin-2-yl)-2-((4-phenyl-1H-pyrazol-3-yl)methyl)-2,9-diazaspiro[5.5]undecan-1-one;
9-(4,6-dimethylpyrimidin-2-yl)-2-((1-methyl-4-phenyl-1H-pyrazol-3-yl)methyl)-2,9-diazaspiro[5.5]undecan-1-one;
9-(4,6-dimethylpyrimidin-2-yl)-2-((2-methyl-5-phenyloxazol-4-yl)methyl)-2,9-diazaspiro[5.5]undecan-1-one;
9-(4,6-dimethylpyrimidin-2-yl)-2-((2-methyl-5-phenylthiazol-4-yl)methyl)-2,9-diazaspiro[5.5]undecan-1-one;
9-(4,6-dimethylpyrimidin-2-yl)-2-((5-(3-methoxyphenyl)-2-methylthiazol-4-yl)methyl)-2,9-diazaspiro[5.5]undecan-1-one;
2-((1H-indol-3-yl)methyl)-9-(4-ethyl-6-methylpyrimidin-2-yl)-2,9-diazaspiro[5.5]undecan-1-one;
2-((1H-indazol-3-yl)methyl)-9-(4-methoxy-6-methylpyrimidin-2-yl)-2,9-diazaspiro[5.5]undecan-1-one;
2-((1H-indol-3-yl)methyl)-9-(4-(dimethylamino)-6-methylpyrimidin-2-yl)-2,9-diazaspiro[5.5]undecan-1-one;
and 9-(4-methoxypyrimidin-2-yl)-2-(2-(3-methyl-1,2,4-oxadiazol-5-yl)benzyl)-2,9-diazaspiro[5.5]undecan-1-one;
Methyl 3-((9-(4,6-dimethylpyrimidin-2-yl)-1-oxo-2,9-diazaspiro[5.5]undecan-2-yl)methyl)-1H-indole-5-carboxylate;
9-(4,6-dimethylpyrimidin-2-yl)-2-((5-(methoxymethyl)-1H-indol-3-yl)methyl)-2,9-diazaspiro[5.5]undecan-1-one;
2-((5-(1H-pyrazol-1-yl)-1H-indol-3-yl)methyl)-9-(4,6-dimethylpyrimidin-2-yl)-2,9-diazaspiro[5.5]undecan-1-one;
2-((5-bromo-2-methyl-2H-1,2,3-triazol-4-yl)methyl)-9-(4,6-dimethylpyrimidin-2-yl)-2,9-diazaspiro[5.5]undecan-1-one;
9-(4,6-dimethylpyrimidin-2-yl)-2-O-(3-(methoxymethyl)phenyl)-2-methyl-2H-1,2,3-triazol-4-yl)methyl)-2,9-diazaspiro[5.5]undecan-1-one;
9-(4,6-dimethylpyrimidin-2-yl)-2-((5-(6-methoxypyridin-2-yl)-2-methyl-2H-1,2,3-triazol-4-yl)methyl)-2,9-diazaspiro[5.5]undecan-1-one;
9-(4,6-dimethylpyrimidin-2-yl)-2-((5-(5-methoxypyridin-3-yl)-2-methyl-2H-1,2,3-triazol-4-yl)methyl)-2,9-diazaspiro[5.5]undecan-1-one;
9-(4,6-dimethylpyrimidin-2-yl)-2-((5-(2-methoxypyridin-4-yl)-2-methyl-2H-1,2,3-triazol-4-yl)methyl)-2,9-diazaspiro[5.5]undecan-1-one;
9-(4,6-dimethylpyrimidin-2-yl)-2-((5-(3-(methoxymethyl)phenyl)oxazol-4-yl)methyl)-2,9-diazaspiro[5.5]undecan-1-one;
9-(4,6-dimethylpyrimidin-2-yl)-2-((5-(3-(methoxymethyl)phenyl)-2-methyloxazol-4-yl)methyl)-2,9-diazaspiro[5.5]undecan-1-one;
9-(4,6-dimethylpyrimidin-2-yl)-2-(3-(methoxymethyl)benzyl)-2,9-diazaspiro[5.5]undecan-1-one;
9-(4,6-dimethylpyrimidin-2-yl)-2-(2-(methoxymethyl)benzyl)-2,9-diazaspiro[5.5]undecan-1-one; and
9-(4-methoxy-6-methylpyrimidin-2-yl)-2-(3-(methoxymethyl)benzyl)-2,9-diazaspiro[5.5]undecan-1-one.

### EMBODIMENT 8

A pharmaceutical composition comprising a therapeutically effective amount of a compound according to any one of embodiments 1 to 7 in free form or in salt form or in pharmaceutically acceptable salt form and one or more pharmaceutically acceptable carriers.

### EMBODIMENT 9

A combination comprising a therapeutically effective amount of the compound according to any one of embodiments 1 to 7 in free form or in salt form or in pharmaceutically acceptable salt form and one or more therapeutically active agents.

### EMBODIMENT 10

A method of inhibiting orexin receptor activity in a subject, wherein the method comprises administering to the subject a therapeutically effective amount of the compound according to any one of embodiments 1 to 7 in free form or in salt form or in pharmaceutically acceptable salt form.

### EMBODIMENT 11

A method of treating a disorder or a disease in a subject mediated by orexin receptors, wherein the method comprises administering to the subject a therapeutically effective amount of the compound according to any one of embodiments 1 to 7 in free form or in salt form or in pharmaceutically acceptable salt form.

Embodiment 12. A compound according to any one of embodiments 1 to 7 in free form or in salt form or in pharmaceutically acceptable salt form, for use as a medicament.

### EMBODIMENT 13

Use of a compound according to any one of embodiments 1 to 7 in free form or in salt form or in pharmaceutically acceptable salt form, for the treatment of a disorder or disease in a subject mediated by orexin receptors.

### EMBODIMENT 14

Use of a compound according to any one of embodiments 1 to 7 in free form or in salt form or in pharmaceutically acceptable salt form, for the treatment of a disorder or disease in a subject characterized by an abnormal activity of orexin receptors.

1. A compound of the formula I 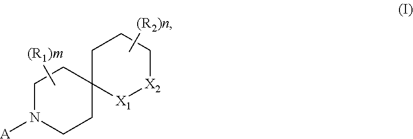
wherein
A is a five- to six-membered aromatic ring system which may contain from 1 to 4 hetero atoms selected from nitrogen, oxygen and sulfur, wherein the ring system is substituted once or more than once by R₃, and wherein a substituent on a nitrogen in a heterocyclic ring system may not be halogen;
each R₃ independently is halogen; cyano; hydroxy; amino; or —X₃—R₄;
each X₃ independently is selected from bond; carbonyl; oxygen; sulfur; —S(O)—; —S(O)₂—; amino, which may be substituted by C₁₋₄alkyl; —NH—C(O)— and —C(O)—NH—;
each R₄ independently is C₁₋₆alkyl; C₁₋₆halogenalkyl; C₁₋₆cyanoalkyl; C₁₋₆hydroxyalkyl; C₁₋₄alkoxy-C₁₋₆alkyl; C₁₋₆-aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkinyl; C₂₋₆halogenalkinyl; C₃₋₆cycloalkyl, wherein one carbon atom may be exchanged by oxygen or amino, which in turn may be substituted by C₁₋₄alkyl, and wherein the C₃₋₆cycloalkyl may be attached directly to X₃ or via a C₁₋₂alkylene, and wherein the C₃₋₆cycloalkyl in turn may be substituted by halogen or C₁₋₄alkyl;
m is 0, 1, 2, 3, 4, 5 or 6;
n is 0, 1, 2, 3, 4, 5 or 6;
each R₁ or R₂ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;
—X₁— is —C(O)— and —X₂— is —N(L-B)—;
or —X₁— is —N(L-B)— and —X₂— is —C(O)—;
L is —C(R₆)₂—;
each R₆ independently is hydrogen, C₁₋₆allyl, C₁₋₆halogenalkyl, C₃₋₇cycloalkyl or C₃₋₇cycloalkyl(C₁₋₄alkyl);
or two R₆ together with the carbon atom to which they are bound form a C₃₋₄cycloalkyl;
B is a five- to six-membered monocyclic or eight- to ten-membered fused biycyclic aromatic ring system which may contain from 1 to 4 hetero atoms selected from nitrogen, oxygen and sulfur, wherein the ring system may be substituted once or more than once by R₇, and wherein a substituent on a nitrogen in a heterocyclic ring system may not be halogen;
each R₇ independently is C₁₋₆alkyl, C₁₋₆halogenalkyl, C₁₋₆alkoxy, C₁₋₄alkoxyC₁₋₆alkyl, C₁₋₄alkoxycarbonyl, C₁₋₆halogenalkoxy, halogen, cyano or a three- to seven-membered monocyclic ring system which may be aromatic, saturated or unsaturated non-aromatic and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₄alkoxyC₁₋₆alkyl, C₁₋₆halogenalkoxy, halogen or cyano, and wherein a substituent on a nitrogen in a heterocyclic ring system may not be halogen;
or two R₇ at adjacent ring atoms form together with said ring atoms a fused five- to seven-membered unsaturated non-aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may in turn be substituted once or more than once by R₈, and wherein a substituent on a nitrogen in a heterocyclic ring system may not be halogen; and wherein each R₈ independently is halogen or C₁₋₆alkyl, or two R₈ at the same ring atom together are oxo;
in free form or in salt form or in pharmaceutically acceptable salt form.

2. The compound of formula I according to claim 1 in free form or in salt form or in pharmaceutically acceptable salt form, wherein —X₁— is —C(O)— and —X₂— is —N(L-B)—.

3. The compound of formula I according to claim 1 in free form or in salt form or in pharmaceutically acceptable salt form, wherein m and n are both 0; and each R₆ is hydrogen.

4. The compound of formula I according to claim 1 in free form or in salt form or in pharmaceutically acceptable salt form, wherein A is pyrid-2-yl being substituted in the 4-position and/or in the 6-position by R₃;
or A is pyrimidin-2-yl being substituted in the 4-position or in the 4-position and in the 6-position by R₃;
each R₃ independently is halogen; cyano; hydroxy; amino; or —X₃—R₄;
each X₃ independently is selected from bond; oxygen and amino, which may be substituted by C₁₋₄alkyl; and
each R₄ independently is C₁₋₄alkyl; C₁₋₄halogenalkyl; C₁₋₄-aminoalkyl; C₁₋₄alkylamino-C₁₋₄alkyl; di(C₁₋₄alkyl)amino-C₁₋₄alkyl; or C₃₋₄cycloalkyl.

5. The compound of formula I according claim 1 in free form or in salt form or in pharmaceutically acceptable salt form, wherein B is indol-3-yl which may be substituted once or more than once by R₇, wherein a substituent on the nitrogen of the indol-3-yl may not be halogen; and each R₇ independently is C₁₋₆alkyl, C₁₋₆alkoxy, C₁₋₄alkoxyC₁₋₆alkyl, C₁₋₄alkoxycarbonyl, C₁₋₆halogenalkyl or halogen.

6. The compound of formula I according to claim 1 in free form or in salt form or in pharmaceutically acceptable salt form, wherein B is a six-membered monocyclic aromatic ring system which may contain 1 to 2 nitrogen atoms,
wherein the ring system is substituted once by R₇ₐ, and wherein the ring system may be further substituted once or more than once by R_{7b};
R₇ₐ is a five-membered monocyclic aromatic ring system, which contains from 1 to 4 hetero atoms selected from nitrogen, oxygen and sulfur, and wherein the ring system may in turn be substituted once or more than once by C₁₋₆allyl, C₁₋₆halogenalkyl, C₁₋₆alkoxy, C₁₋₆halogenalkoxy, halogen or cyano, and wherein a substituent on a nitrogen in a heterocyclic ring system may not be halogen; and
each R_{7b} independently is C₁₋₆allyl, C₁₋₆halogenalkyl, C₁₋₆alkoxy, C₁₋₆halogenalkoxy, halogen or cyano.

7. A compound of formula I according to claim 1 in free form or in salt form or in pharmaceutically acceptable salt form, wherein said compound is selected from the group consisting of
9-(4,6-dimethylpyrimidin-2-yl)-2-(2-(3-methyl-1,2,4-oxadiazol-5-yl)benzyl)-2,9-diazaspiro[5.5]undecan-1-one;
2-(2-(2H-1,2,3-triazol-2-yl)benzyl)-9-(4,6-dimethylpyrimidin-2-yl)-2,9-diazaspiro[5.5]undecan-1-one;
1-((1H-indol-4-yl)methyl)-9-(4,6-dimethylpyrimidin-2-yl)-1,9-diazaspiro[5.5]undecan-2-one;
2-((1H-indol-3-yl)methyl)-9-(4-methoxy-6-methylpyrimidin-2-yl)-2,9-diazaspiro[5.5]undecan-1-one;
2-((1H-indol-3-yl)methyl)-9-(4,6-dimethylpyrimidin-2-yl)-2,9-diazaspiro[5.5]undecan-1-one;
9-(4,6-dimethylpyrimidin-2-yl)-2-((1-methyl-1H-indol-3-yl)methyl)-2,9-diazaspiro[5.5]undecan-1-one;
1-((1H-indol-3-yl)methyl)-9-(6-methylpyrazin-2-yl)-1,9-diazaspiro[5.5]undecan-2-one;
2-((1H-pyrrolo[2,3-b]pyridin-3-yl)methyl)-9-(6-methoxypyrazin-2-yl)-2,9-diazaspiro[5.5]undecan-1-one;
2-((1H-indol-3-yl)methyl)-9-(6-methylpyridin-2-yl)-2,9-diazaspiro[5.5]undecan-1-one;
2-(2-((1H-indol-3-yl)methyl)-1-oxo-2,9-diazaspiro[5.5]undecan-9-yl)isonicotinonitrile;
2-((1H-indol-3-yl)methyl)-9-(6-methylpyrazin-2-yl)-2,9-diazaspiro[5.5]undecan-1-one;
2-((1H-indol-3-yl)methyl)-9-(4-methylpyrimidin-2-yl)-2,9-diazaspiro[5.5]undecan-1-one;
2-((1H-indol-3-yl)methyl)-9-(4-methoxypyrimidin-2-yl)-2,9-diazaspiro[5.5]undecan-1-one;
6-(1-((1H-indol-4-yl)methyl)-2-oxo-1,9-diazaspiro[5.5]undecan-9-yl)nicotinonitrile;
9-(4,6-dimethylpyrimidin-2-yl)-2-(2-(furan-2-yl)benzyl)-2,9-diazaspiro[5.5]undecan-1-one;
1-(2-(2H-1,2,3-triazol-2-yl)benzyl)-9-(4-methylpyrimidin-2-yl)-1,9-diazaspiro[5.5]undecan-2-one;
2-((1H-indol-3-yl)methyl)-9-(5-methyl-1,3,4-oxadiazol-2-yl)-2,9-diazaspiro[5.5]undecan-1-one;
2-((1H-pyrrolo[2,3-b]pyridin-3-yl)methyl)-9-(6-chloropyrazin-2-yl)-2,9-diazaspiro[5.5]undecan-1-one;
2-((1H-pyrrolo[2,3-b]pyridin-3-yl)methyl)-9-(6-methylpyrazin-2-yl)-2,9-diazaspiro[5.5]undecan-1-one;
2-((2,3-dihydrobenzo[b][1,4]dioxin-5-yl)methyl)-9-(4,6-dimethylpyrimidin-2-yl)-2,9-diazaspiro[5.5]undecan-1-one;
9-(6-chloropyrazin-2-yl)-2-((2,3-dihydrobenzo[b][1,4]dioxin-5-yl)methyl)-2,9-diazaspiro[5.5]undecan-1-one;
9-(4,6-dimethylpyrimidin-2-yl)-2-(3-(5-methyl-1,2,4-oxadiazol-3-yl)benzyl)-2,9-diazaspiro[5.5]undecan-1-one;
9-(4,6-dimethylpyrimidin-2-yl)-2-((5-methyl-3-phenylisoxazol-4-yl)methyl)-2,9-diazaspiro[5.5]undecan-1-one;
2-(2-(2H-1,2,3-triazol-2-yl)benzyl)-9-(4-methylpyrimidin-2-yl)-2,9-diazaspiro[5.5]undecan-1-one;
2-(2-(2-(2H-1,2,3-triazol-2-yl)benzyl)-1-oxo-2,9-diazaspiro[5.5]undecan-9-yl)-6-methylpyrimidine-4-carbonitrile;
6-(2-(2-(2H-1,2,3-triazol-2-yl)benzyl)-1-oxo-2,9-diazaspiro[5.5]undecan-9-yl)picolinonitrile;
2-(2-(2-(2H-1,2,3-triazol-2-yl)benzyl)-1-oxo-2,9-diazaspiro[5.5]undecan-9-yl)isonicotinonitrile;
2-methyl-6-(1-oxo-2-(3-(pyrimidin-2-yl)benzyl)-2,9-diazaspiro[5.5]undecan-9-yl)isonicotinonitrile;
2-methyl-6-(2-(3-(5-methyloxazol-2-yl)benzyl)-1-oxo-2,9-diazaspiro[5.5]undecan-9-yl)isonicotinonitrile;
9-(4-methylpyrimidin-2-yl)-2-(3-(pyrimidin-2-yl)benzyl)-2,9-diazaspiro[5.5]undecan-1-one;
9-(4,6-dimethylpyrimidin-2-yl)-2-(3-(pyrimidin-2-yl)benzyl)-2,9-diazaspiro[5.5]undecan-1-one;
6-methyl-2-(1-oxo-2-(3-(pyrimidin-2-yl)benzyl)-2,9-diazaspiro[5.5]undecan-9-yl)pyrimidine-4-carbonitrile;
2-(1-oxo-2-(3-(pyrimidin-2-yl)benzyl)-2,9-diazaspiro[5.5]undecan-9-yl)pyrimidine-4-carbonitrile;
6-(1-oxo-2-(3-(pyrimidin-2-yl)benzyl)-2,9-diazaspiro[5.5]undecan-9-yl)picolinonitrile;
2-(1-oxo-2-(3-(pyrimidin-2-yl)benzyl)-2,9-diazaspiro[5.5]undecan-9-yl)isonicotinonitrile;
9-(4,6-dimethylpyrimidin-2-yl)-2-(3-(5-methyloxazol-2-yl)benzyl)-2,9-diazaspiro[5.5]undecan-1-one;
6-methyl-2-(2-(3-(5-methyloxazol-2-yl)benzyl)-1-oxo-2,9-diazaspiro[5.5]undecan-9-yl)pyrimidine-4-carbonitrile;
2-(3-(5-methyloxazol-2-yl)benzyl)-9-(4-methylpyrimidin-2-yl)-2,9-diazaspiro[5.5]undecan-1-one;
6-(2-(3-(5-methyloxazol-2-yl)benzyl)-1-oxo-2,9-diazaspiro[5.5]undecan-9-yl)picolinonitrile;
2-(2-(3-(5-methyloxazol-2-yl)benzyl)-1-oxo-2,9-diazaspiro[5.5]undecan-9-yl)isonicotinonitrile;
1-((1H-indol-4-yl)methyl)-9-(6-methylpyrazin-2-yl)-1,9-diazaspiro[5.5]undecan-2-one;
1-((1H-indol-4-yl)methyl)-9-(4-methylpyrimidin-2-yl)-1,9-diazaspiro[5.5]undecan-2-one;
1-((1H-indol-4-yl)methyl)-9-(6-methoxypyrazin-2-yl)-1,9-diazaspiro[5.5]undecan-2-one;
1-((1H-indol-4-yl)methyl)-9-(6-chloropyrazin-2-yl)-1,9-diazaspiro[5.5]undecan-2-one;
2-((1H-indazol-3-yl)methyl)-9-(4,6-dimethylpyrimidin-2-yl)-2,9-diazaspiro[5.5]undecan-1-one;
2-((1H-indol-3-yl)methyl)-9-(4-(dimethylamino)pyrimidin-2-yl)-2,9-diazaspiro[5.5]undecan-1-one;
2-((1H-indol-3-yl)methyl)-9-(2-methoxypyrimidin-4-yl)-2,9-diazaspiro[5.5]undecan-1-one;
2-((1H-indol-3-yl)methyl)-9-(2-chloropyrimidin-4-yl)-2,9-diazaspiro[5.5]undecan-1-one;
2-((1H-indol-3-yl)methyl)-9-(4-(trifluoromethyl)pyrimidin-2-yl)-2,9-diazaspiro[5.5]undecan-1-one;
2-((1H-indol-3-yl)methyl)-9-(4-isopropylpyrimidin-2-yl)-2,9-diazaspiro[5.5]undecan-1-one;
2-((1H-indol-3-yl)methyl)-9-(4-chloropyrimidin-2-yl)-2,9-diazaspiro[5.5]undecan-1-one;
2-((1H-indol-3-yl)methyl)-9-(4-ethoxypyrimidin-2-yl)-2,9-diazaspiro[5.5]undecan-1-one;
9-(6-chloropyrazin-2-yl)-2-((1-methyl-1H-indol-3-yl)methyl)-2,9-diazaspiro[5.5]undecan-1-one;
9-(4,6-dimethylpyrimidin-2-yl)-2-((1-ethyl-1H-indol-3-yl)methyl)-2,9-diazaspiro[5.5]undecan-1-one;
1-((1H-indol-3-yl)methyl)-9-(6-methoxypyrazin-2-yl)-1,9-diazaspiro[5.5]undecan-2-one;
2-((2,3-dihydrobenzo[b][1,4]dioxin-5-yl)methyl)-9-(6-methoxypyrazin-2-yl)-2,9-diazaspiro[5.5]undecan-1-one;
2-((1H-indol-3-yl)methyl)-9-m-tolyl-2,9-diazaspiro[5.5]undecan-1-one;
2-((1H-indol-3-yl)methyl)-9-(5-methylpyridin-3-yl)-2,9-diazaspiro[5.5]undecan-1-one;
2-((1H-indol-3-yl)methyl)-9-(3,4-dimethoxyphenyl)-2,9-diazaspiro[5.5]undecan-1-one;
2-((1H-indol-3-yl)methyl)-9-(3-methoxyphenyl)-2,9-diazaspiro[5.5]undecan-1-one;
2-((1H-indol-3-yl)methyl)-9-(2-methoxypyridin-4-yl)-2,9-diazaspiro[5.5]undecan-1-one;
2-((1H-indol-3-yl)methyl)-9-(5-methoxypyridin-3-yl)-2,9-diazaspiro[5.5]undecan-1-one;
2-((1H-indol-3-yl)methyl)-9-(2,6-dimethylpyridin-4-yl)-2,9-diazaspiro[5.5]undecan-1-one;
2-((1H-indol-3-yl)methyl)-9-(5,6-dimethylpyridin-2-yl)-2,9-diazaspiro[5.5]undecan-1-one;
2-((1H-indol-3-yl)methyl)-9-(6-methoxypyridin-2-yl)-2,9-diazaspiro[5.5]undecan-1-one;
2-((1H-indol-3-yl)methyl)-9-(3-methylpyridin-2-yl)-2,9-diazaspiro[5.5]undecan-1-one;
2-((1H-indol-3-yl)methyl)-9-(4-methylpyridin-2-yl)-2,9-diazaspiro[5.5]undecan-1-one;
2-((1H-indol-3-yl)methyl)-9-(5-methylpyridin-2-yl)-2,9-diazaspiro[5.5]undecan-1-one;
6-(2-((1H-indol-3-yl)methyl)-1-oxo-2,9-diazaspiro[5.5]undecan-9-yl)nicotinonitrile;
2-((1H-indol-3-yl)methyl)-9-(2-methylpyrimidin-4-yl)-2,9-diazaspiro[5.5]undecan-1-one;
2-((1H-indol-3-yl)methyl)-9-(2,6-dimethylpyrimidin-4-yl)-2,9-diazaspiro[5.5]undecan-1-one;
2-((1H-indol-3-yl)methyl)-9-(5-methylpyrazin-2-yl)-2,9-diazaspiro[5.5]undecan-1-one;
2-((1H-indol-3-yl)methyl)-9-(6-(methoxymethyl)pyridin-2-yl)-2,9-diazaspiro[5.5]undecan-1-one;
9-(4,6-dimethylpyridin-2-yl)-2-(3-(pyrimidin-2-yl)benzyl)-2,9-diazaspiro[5.5]undecan-1-one;
2-(2-((1H-indol-3-yl)methyl)-1-oxo-2,9-diazaspiro[5.5]undecan-9-yl)-6-methylpyrimidine-4-carbonitrile;
2-(2-((1H-indol-3-yl)methyl)-1-oxo-2,9-diazaspiro[5.5]undecan-9-yl)pyrimidine-4,6-dicarbonitrile;
6-(2-((1H-indol-3-yl)methyl)-1-oxo-2,9-diazaspiro[5.5]undecan-9-yl)pyrazine-2-carbonitrile;
2-(2-((1H-indol-3-yl)methyl)-1-oxo-2,9-diazaspiro[5.5]undecan-9-yl)-6-methylisonicotinonitrile;
2-((1H-indol-3-yl)methyl)-9-(4-ethylpyrimidin-2-yl)-2,9-diazaspiro[5.5]undecan-1-one;
2-(2-((1H-indol-3-yl)methyl)-1-oxo-2,9-diazaspiro[5.5]undecan-9-yl)pyrimidine-4-carbonitrile;
2-((1H-indol-3-yl)methyl)-9-(4,5-dimethylpyrimidin-2-yl)-2,9-diazaspiro[5.5]undecan-1-one;
2-((1H-indol-3-yl)methyl)-9-(6-methoxypyrazin-2-yl)-2,9-diazaspiro[5.5]undecan-1-one;
6-(2-((1H-indol-3-yl)methyl)-1-oxo-2,9-diazaspiro[5.5]undecan-9-yl)picolinonitrile;
2-((1H-indol-3-yl)methyl)-9-(6-hydroxypyridin-2-yl)-2,9-diazaspiro[5.5]undecan-1-one;
2-((1H-indol-3-yl)methyl)-9-(5-methyl-1,3,4-thiadiazol-2-yl)-2,9-diazaspiro[5.5]undecan-1-one;
2-((1H-indol-3-yl)methyl)-9-(3,6-dimethylpyrazin-2-yl)-2,9-diazaspiro[5.5]undecan-1-one;
2-((1H-indol-3-yl)methyl)-9-(6-methoxypyrimidin-4-yl)-2,9-diazaspiro[5.5]undecan-1-one;
2-((1H-indol-3-yl)methyl)-9-(4,6-dimethoxypyrimidin-2-yl)-2,9-diazaspiro[5.5]undecan-1-one;
2-((1H-indol-3-yl)methyl)-9-(5-(trifluoromethyl)pyridin-2-yl)-2,9-diazaspiro[5.5]undecan-1-one;
9-(4-methylpyrimidin-2-yl)-1-(4-(pyrimidin-2-yl)benzyl)-1,9-diazaspiro[5.5]undecan-2-one;
2-(1-((1H-indol-4-yl)methyl)-2-oxo-1,9-diazaspiro[5.5]undecan-9-yl)isonicotinonitrile;
6-(1-((1H-indol-4-yl)methyl)-2-oxo-1,9-diazaspiro[5.5]undecan-9-yl)picolinonitrile;
9-(4,6-dimethylpyrimidin-2-yl)-2-(2-(oxazol-2-yl)benzyl)-2,9-diazaspiro[5.5]undecan-1-one;
1-(3-(2H-1,2,3-triazol-2-yl)benzyl)-9-(4-methylpyrimidin-2-yl)-1,9-diazaspiro[5.5]undecan-2-one;
9-(4-methylpyrimidin-2-yl)-1-((5-phenyl-1,3,4-oxadiazol-2-yl)methyl)-1,9-diazaspiro[5.5]undecan-2-one;
1-((2-methyl-1H-indol-4-yl)methyl)-9-(4-methylpyrimidin-2-yl)-1,9-diazaspiro[5.5]undecan-2-one;
1-(3-(3-methyl-1,2,4-oxadiazol-5-yl)benzyl)-9-(4-methylpyrimidin-2-yl)-1,9-diazaspiro[5.5]undecan-2-one;
9-(4-methylpyrimidin-2-yl)-1-((6-(pyrrolidin-1-yl)pyridin-2-yl)methyl)-1,9-diazaspiro[5.5]undecan-2-one;
1-(2-(3-methyl-1,2,4-oxadiazol-5-yl)benzyl)-9-(4-methylpyrimidin-2-yl)-1,9-diazaspiro[5.5]undecan-2-one;
2-((1H-indol-3-yl)methyl)-9-(2-methylpyridin-4-yl)-2,9-diazaspiro[5.5]undecan-1-one;
4-fluoro-3-((9-(4-methylpyrimidin-2-yl)-2-oxo-1,9-diazaspiro[5.5]undecan-1-yl)methyl)benzonitrile;
2-((1H-indol-3-yl)methyl)-9-(5-fluoro-4-methylpyrimidin-2-yl)-2,9-diazaspiro[5.5]undecan-1-one;
9-(4,6-dimethylpyrimidin-2-yl)-2-((7-methyl-1H-indol-3-yl)methyl)-2,9-diazaspiro[5.5]undecan-1-one;
9-(4,6-dimethylpyrimidin-2-yl)-2-((5-methyl-1H-indol-3-yl)methyl)-2,9-diazaspiro[5.5]undecan-1-one;
3-((9-(4,6-dimethylpyrimidin-2-yl)-1-oxo-2,9-diazaspiro[5.5]undecan-2-yl)methyl)-1H-indole-5-carbonitrile;
9-(4,6-dimethylpyrimidin-2-yl)-2-((4-phenyl-1H-pyrazol-3-yl)methyl)-2,9-diazaspiro[5.5]undecan-1-one;
9-(4,6-dimethylpyrimidin-2-yl)-2-((1-methyl-4-phenyl-1H-pyrazol-3-yl)methyl)-2,9-diazaspiro[5.5]undecan-1-one;
9-(4,6-dimethylpyrimidin-2-yl)-2-((2-methyl-5-phenyloxazol-4-yl)methyl)-2,9-diazaspiro[5.5]undecan-1-one;
9-(4,6-dimethylpyrimidin-2-yl)-2-((2-methyl-5-phenylthiazol-4-yl)methyl)-2,9-diazaspiro[5.5]undecan-1-one;
9-(4,6-dimethylpyrimidin-2-yl)-2-((5-(3-methoxyphenyl)-2-methylthiazol-4-yl)methyl)-2,9-diazaspiro[5.5]undecan-1-one;
2-((1H-indol-3-yl)methyl)-9-(4-ethyl-6-methylpyrimidin-2-yl)-2,9-diazaspiro[5.5]undecan-1-one;
2-((1H-indazol-3-yl)methyl)-9-(4-methoxy-6-methylpyrimidin-2-yl)-2,9-diazaspiro[5.5]undecan-1-one;
2-((1H-indol-3-yl)methyl)-9-(4-(dimethylamino)-6-methylpyrimidin-2-yl)-2,9-diazaspiro[5.5]undecan-1-one;
and 9-(4-methoxypyrimidin-2-yl)-2-(2-(3-methyl-1,2,4-oxadiazol-5-yl)benzyl)-2,9-diazaspiro[5.5]undecan-1-one;
Methyl 3-((9-(4,6-dimethylpyrimidin-2-yl)-1-oxo-2,9-diazaspiro[5.5]undecan-2-yl)methyl)-1H-indole-5-carboxylate;
9-(4,6-dimethylpyrimidin-2-yl)-2-((5-(methoxymethyl)-1H-indol-3-yl)methyl)-2,9-diazaspiro[5.5]undecan-1-one;
2-((5-(1H-pyrazol-1-yl)-1H-indol-3-yl)methyl)-9-(4,6-dimethylpyrimidin-2-yl)-2,9-diazaspiro[5.5]undecan-1-one;
2-((5-bromo-2-methyl-2H-1,2,3-triazol-4-yl)methyl)-9-(4,6-dimethylpyrimidin-2-yl)-2,9-diazaspiro[5.5]undecan-1-one;
9-(4,6-dimethylpyrimidin-2-yl)-2-((5-(3-(methoxymethyl)phenyl)-2-methyl-2H-1,2,3-triazol-4-yl)methyl)-2,9-diazaspiro[5.5]undecan-1-one;
9-(4,6-dimethylpyrimidin-2-yl)-2-((5-(6-methoxypyridin-2-yl)-2-methyl-2H-1,2,3-triazol-4-yl)methyl)-2,9-diazaspiro[5.5]undecan-1-one;
9-(4,6-dimethylpyrimidin-2-yl)-2-((5-(5-methoxypyridin-3-yl)-2-methyl-2H-1,2,3-triazol-4-yl)methyl)-2,9-diazaspiro[5.5]undecan-1-one;
9-(4,6-dimethylpyrimidin-2-yl)-2-((5-(2-methoxypyridin-4-yl)-2-methyl-2H-1,2,3-triazol-4-yl)methyl)-2,9-diazaspiro[5.5]undecan-1-one;
9-(4,6-dimethylpyrimidin-2-yl)-2-((5-(3-(methoxymethyl)phenyl)oxazol-4-yl)methyl)-2,9-diazaspiro[5.5]undecan-1-one;
9-(4,6-dimethylpyrimidin-2-yl)-2-((5-(3-(methoxymethyl)phenyl)-2-methyloxazol-4-yl)methyl)-2,9-diazaspiro[5.5]undecan-1-one;
9-(4,6-dimethylpyrimidin-2-yl)-2-(3-(methoxymethyl)benzyl)-2,9-diazaspiro[5.5]undecan-1-one;
9-(4,6-dimethylpyrimidin-2-yl)-2-(2-(methoxymethyl)benzyl)-2,9-diazaspiro[5.5]undecan-1-one; and
9-(4-methoxy-6-methylpyrimidin-2-yl)-2-(3-(methoxymethyl)benzyl)-2,9-diazaspiro[5.5]undecan-1-one.

8. A pharmaceutical composition comprising a therapeutically effective amount of a compound according to claim 1 in free form or in salt form or in pharmaceutically acceptable salt form and one or more pharmaceutically acceptable carriers.

9. A combination comprising a therapeutically effective amount of the compound according to claim 1 in free form or in salt form or in pharmaceutically acceptable salt form and one or more therapeutically active agents.

10. A method of inhibiting orexin receptor activity in a subject, wherein the method comprises administering to the subject a therapeutically effective amount of the compound according to claim 1 in free form or in salt form or in pharmaceutically acceptable salt form.

11. A method of treating a disorder or a disease in a subject mediated by orexin receptors, wherein the method comprises administering to the subject a therapeutically effective amount of the compound according to claim 1 in free form or in salt form or in pharmaceutically acceptable salt form.

12-14. (canceled)
